(19) United States
(12) Patent Application Publication
(10) Pub. No.: US 2024/0360166 A1
(43) Pub. Date: Oct. 31, 2024

(54) SALTS AND SOLID FORMS OF A COMPOUND HAVING GLP-1 AGONIST ACTIVITY

(71) Applicant: Gasherbrum Bio, Inc., South San Francisco, CA (US)

(72) Inventors: Libo CHEN, Shanghai (CN); Xiaodong GU, Shanghai (CN); Xinglong JIANG, Hillsborough, NJ (US)

(21) Appl. No.: 18/620,978

(22) Filed: Mar. 28, 2024

Related U.S. Application Data
(63) Continuation of application No. PCT/CN2023/138803, filed on Dec. 14, 2023.

(30) Foreign Application Priority Data
Dec. 15, 2022 (WO) PCT/CN2022/139277

(51) Int. Cl. C07F 9/6561; A61K 31/675; C07C 279/14

(52) U.S. Cl. CPC C07F 9/6561; A61K 31/675; C07C 279/14; C07B 2200/13

(57) ABSTRACT

The present disclosure relates to salts and solid forms of a compound that are GLP-1 agonists, and its use as a therapeutic agent for treating diseases disorders, or conditions associated with GLP-1, such as type 2 diabetes mellitus (T2DM).

### CROSS REFERENCE TO RELATED APPLICATIONS

This application claims the benefit of International Patent Application Number PCT/CN2022/139277 filed on Dec. 15, 2022, the content of which is hereby incorporated herein in its entirety.

### FIELD

The present disclosure relates generally to salts and solid forms of GLP-1 agonists, pharmaceutical compositions, and methods of use thereof.

### BACKGROUND

The present disclosure relates to salts and solid forms of a compound that are glucagon-like peptide-1 (GLP-1) agonists, and its use as a therapeutic agent for treating diseases, disorders, or conditions associated with GLP-1, such as type 2 diabetes mellitus (T2DM).

### SUMMARY

The present disclosure provides salts and solid forms of Compound I (CAS Registry Number: 2685823-26-9), and co-crystals and solvates thereof. Also described herein are processes for making salts and solid forms of Compound I, pharmaceutical compositions comprising salts or solid forms of Compound I, and methods for using the same, in the treatment of diseases associated with GLP-1.

### BRIEF DESCRIPTION OF THE DRAWINGS

FIG. 1A shows an X-ray powder diffraction (XRPD) of Compound I L-arginine salt Form A.

FIG. 1B shows a differential scanning calorimeter (DSC) curve of Compound I L-arginine salt Form A.

FIG. 1C shows a thermogravimetric analysis (TGA) plot of Compound I L-arginine salt Form A.

FIG. 2A shows an XRPD of Compound I L-arginine salt Form B.

FIG. 2B shows a DSC curve of Compound I L-arginine salt Form B.

FIG. 2C shows a TGA plot of Compound I L-arginine salt Form B.

FIG. 3A shows an XRPD of Compound I L-arginine salt Form C.

FIG. 3B shows a DSC curve of Compound I L-arginine salt Form C.

FIG. 3C shows a TGA plot of Compound I L-arginine salt Form C.

FIG. 4A shows an XRPD of Compound I free acid Form A.

FIG. 4B shows a DSC curve of Compound I free acid Form A.

FIG. 4C shows a TGA plot of Compound I free acid Form A.

FIG. 5A shows an XRPD of Compound I free acid Form B.

FIG. 5B shows a DSC curve of Compound I free acid Form B.

FIG. 5C shows a TGA plot of Compound I free acid Form B.

FIG. 6A shows an XRPD of Compound I free acid Form C.

FIG. 6B shows a DSC curve of Compound I free acid Form C.

FIG. 6C shows a TGA plot of Compound I free acid Form C.

FIG. 7A shows an XRPD of Compound I free acid Form D.

FIG. 7B shows a DSC curve of Compound I free acid Form D.

FIG. 7C shows a TGA plot of Compound I free acid Form D.

FIG. 8A shows an XRPD of Compound I free acid Form E.

FIG. 8B shows a DSC curve of Compound I free acid Form E.

FIG. 8C shows a TGA plot of Compound I free acid Form E.

FIG. 9A shows an XRPD of Compound I free acid Form F.

FIG. 9B shows a DSC curve of Compound I free acid Form F.

FIG. 9C shows a TGA plot of Compound I free acid Form F.

FIG. 10A shows an XRPD of Compound I free acid Form G.

FIG. 10B shows a DSC curve of Compound I free acid Form G.

FIG. 10C shows a TGA plot of Compound I free acid Form G.

FIG. 11A shows an XRPD of Compound I free acid Form H.

FIG. 11B shows a DSC curve of Compound I free acid Form H.

FIG. 11C shows a TGA plot of Compound I free acid Form H.

FIG. 12A shows an XRPD of Compound I free acid Form I.

FIG. 12B shows a DSC curve of Compound I free acid Form I.

FIG. 12C shows a TGA plot of Compound I free acid Form I.

FIG. 13A shows an XRPD of Compound I free acid Form J.

FIG. 13B shows a DSC curve of Compound I free acid Form J.

FIG. 13C shows a TGA plot of Compound I free acid Form J.

FIG. 14A shows an XRPD of Compound I free acid Form K.

FIG. 14B shows a DSC curve of Compound I free acid Form K.

FIG. 14C shows a TGA plot of Compound I free acid Form K.

FIG. 15A shows an XRPD of Compound I free acid Form L.

FIG. 15B shows a DSC curve of Compound I free acid Form L.

FIG. 15C shows a TGA plot of Compound I free acid Form L.

FIG. 16A shows and XRPD of Compound I free acid Form M.

FIG. 17A shows an XRPD of Compound I free acid Form N.

FIG. 17B shows a DSC curve of Compound I free acid Form N.

FIG. 17C shows a TGA plot of Compound I free acid Form N.

FIG. 18A shows an XRPD of Compound I sodium salt Form A.

FIG. 18B shows a DSC curve of Compound I sodium salt Form A.

FIG. 18C shows a TGA plot of Compound I sodium salt Form A.

FIG. 19A shows an XRPD of Compound I sodium salt Form B.

FIG. 20A shows an XRPD of Compound I potassium salt Form A.

FIG. 20B shows a DSC curve of Compound I potassium salt Form A.

FIG. 20C shows a TGA plot of Compound I potassium salt Form A.

### DETAILED DESCRIPTION

The compound 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one, designated herein as Compound I, has the following formula:

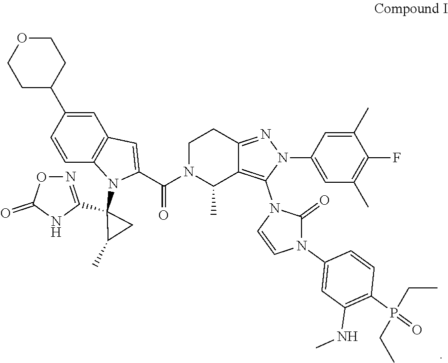 [SMILES: CCP(=O)(CC)c1ccc(-n2ccn(-c3c4c(nn3-c3cc(C)c(F)c(C)c3)CCN(C(=O)c3cc5cc(C6CCOCC6)ccc5n3[C@@]3(c5noc(=O)[nH]5)C[C@@H]3C)[C@H]4C)c2=O)cc1NC]

Compound I is a GLP-1 agonist. The synthesis and method of use thereof is described in PCT International Application Publication No. WO2021/155841 which is incorporated by reference herein in its entirety.

While not intending to be bound by any particular theory, certain solid forms are characterized by physical properties, e.g., stability, solubility, and dissolution rate, appropriate for pharmaceutical and therapeutic dosage forms. Moreover, while not wishing to be bound by any particular theory, certain solid forms are characterized by physical properties (e.g., density, compressibility, hardness, morphology, cleavage, stickiness, solubility, water uptake, electrical properties, thermal behavior, solid-state reactivity, physical stability, and chemical stability) affecting particular processes (e.g., yield, filtration, washing, drying, milling, mixing, tableting, flowability, dissolution, formulation, and lyophilization) which make certain solid forms suitable for the manufacture of a solid dosage form. Such properties can be determined using particular analytical chemical techniques, including solid-state analytical techniques (e.g., X-ray diffraction, microscopy, spectroscopy, and thermal analysis), as described herein.

The identification and selection of a solid form of a pharmaceutical compound are complex, given that a change in solid form may affect a variety of physical and chemical properties, which may provide benefits or drawbacks in processing, formulation, stability, bioavailability, storage, and handling (e.g., shipping), among other important pharmaceutical characteristics. Useful pharmaceutical solids include crystalline solids and amorphous solids, depending on the product and its mode of administration. Amorphous solids are characterized by a lack of long-range structural order, whereas crystalline solids are characterized by structural periodicity. The desired class of pharmaceutical solid depends upon the specific application; amorphous solids are sometimes selected on the basis of, e.g., an enhanced dissolution profile, while crystalline solids may be desirable for properties such as, e.g., physical, or chemical stability.

Whether crystalline or amorphous, solid forms of a pharmaceutical compound include single-component and multiple-component solids. Single-component solids consist essentially of the pharmaceutical compound or active ingredient in the absence of other compounds. Variety among single-component crystalline materials may potentially arise from the phenomenon of polymorphism, wherein multiple three-dimensional arrangements exist for a particular pharmaceutical compound.

Notably, it is not possible to predict a priori if crystalline forms of a compound even exist, let alone how to successfully prepare them (see, e.g., Braga and Grepioni, 2005, “Making crystals from crystals: a green route to crystal engineering and polymorphism,” Chem. Commun.:3635-3645 (with respect to crystal engineering, if instructions are not very precise and/or if other external factors affect the process, the result can be unpredictable); Jones et al., 2006, Pharmaceutical Cocrystals: An Emerging Approach to Physical Property Enhancement,” MRS Bulletin 31:875-879 (At present it is not generally possible to computationally predict the number of observable polymorphs of even the simplest molecules); Price, 2004, “The computational prediction of pharmaceutical crystal structures and polymorphism,” Advanced Drug Delivery Reviews 56:301-319 (“Price”); and Bernstein, 2004, “Crystal Structure Prediction and Polymorphism,” ACA Transactions 39:14-23 (a great deal still needs to be learned and done before one can state with any degree of confidence the ability to predict a crystal structure, much less polymorphic forms)).

The variety of possible solid forms creates potential diversity in physical and chemical properties for a given pharmaceutical compound. The discovery and selection of solid forms are of great importance in the development of an effective, stable, and marketable pharmaceutical product.

### 1. Definitions

As used in the present specification, the following words and phrases are generally intended to have the meanings as set forth below, except to the extent that the context in which they are used indicates otherwise.

The term “comprise” and variations thereof, such as, “comprises” and “comprising” are to be construed in an open, inclusive sense, that is, as “including, but not limited to.” Further, the singular forms “a,” “an,” and “the” include plural references unless the context clearly dictates otherwise. Thus, reference to “the compound” includes a plurality of such compounds, and reference to “the assay” includes reference to one or more assays and equivalents thereof known to those skilled in the art.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±2.5%. In certain other embodiments, the term “about” includes the indicated amount ±1%. Also, to the term “about x” includes description of “x”.

Recitation of numeric ranges of values throughout the disclosure is intended to serve as a shorthand notation of referring individually to each separate value falling within the range inclusive of the values defining the range, and each separate value is incorporated in the specification as it were individually recited herein.

Forms of Compound I or salts, co-crystals, solvates, or hydrates thereof are provided herein. In one embodiment, reference to a form of Compound I or a salt, co-crystal, solvate, or hydrate thereof means that at least 50% to 99% (e.g., at least 50%, at least 55%, at least 60%, at least 65%, at least 70%, at least 75%, at least 80%, at least 85%, at least 90%, at least 95%, or at least 99%) of Compound I or a salt, co-crystal, solvate, or hydrate thereof present in a composition is in the designated form. For instance, in one embodiment, reference to Compound I free acid Form A means that at least 50%, at least 55%, at least 60%, at least 65%, at least 70%, at least 75%, at least 80%, at least 85%, at least 90%, at least 95%, or at least 99% of Compound I, as the free acid, is present in a composition as Form A.

The term “solid form” refers to a type of solid-state material that includes amorphous as well as crystalline forms. The term “crystalline form” refers to polymorphs as well as solvates, hydrates, etc. The term “polymorph” refers to a particular crystal structure having particular physical properties such as X-ray diffraction, melting point, and the like.

The term “co-crystal” refers to a molecular complex of a compound disclosed herein and one or more non-ionized co-crystal formers connected via non-covalent interactions. In some embodiments, the co-crystals disclosed herein may include a non-ionized form of Compound I (e.g., Compound I free form) and one or more non-ionized co-crystal formers, where non-ionized Compound I and the co-crystal former(s) are connected through non-covalent interactions. In some embodiments, co-crystals disclosed herein may include an ionized form of Compound I (e.g., a salt of Compound I) and one or more non-ionized co-crystals formers, where ionized Compound I and the co-crystal former(s) are connected through non-covalent interactions. Co-crystals may additionally be present in anhydrous, solvated or hydrated forms. In certain instances, co-crystals may have improved properties as compared to the parent form (i.e., the free molecule, zwitterion, etc.) or a salt of the parent compound. Improved properties can be increased solubility, increased dissolution, increased bioavailability, increased dose response, decreased hygroscopicity, increased stability, a crystalline form of a normally amorphous compound, a crystalline form of a difficult to salt or unsaltable compound, decreased form diversity, more desired morphology, and the like. Methods for making and characterizing co-crystals are known to those of skill in the art.

The term “co-crystal former” or “co-former” refers to one or more pharmaceutically acceptable bases or pharmaceutically acceptable acids disclosed herein in association with Compound I, or any other compound disclosed herein.

The term “solvate” refers to a complex formed by combination of solvent molecules with molecules or ions of the solute. The solvent can be an organic compound, an inorganic compound, or a mixture of both. As used herein, the term “solvate” includes a “hydrate” (i.e., a complex formed by combination of water molecules with molecules or ions of the solute), hemi-hydrate, channel hydrate, etc. The term “hetero-solvate” refers to a complex includes a mixture of one or more different organic solvents and/or water. Some examples of solvents include, but are not limited to, methanol, N,N-dimethylformamide, tetrahydrofuran, dimethylsulfoxide, and water.

The term “desolvated” refers to a Compound I form that is a solvate as described herein, and from which solvent molecules have been partially or completely removed. Desolvation techniques to produce desolvated forms include, without limitation, exposure of a Compound I form (solvate) to a vacuum, subjecting the solvate to elevated temperature, exposing the solvate to a stream of gas, such as air or nitrogen, or any combination thereof. Thus, a desolvated Compound I form can be anhydrous, i.e., completely without solvent molecules, or partially solvated wherein solvent molecules are present in stoichiometric or non-stoichiometric amounts.

The term “amorphous” refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterized by a change of state, typically second order (glass transition).

Any formula or structure given herein, including Compound I, is also intended to represent unlabeled forms as well as isotopically labeled forms of the compounds. It is understood that for any given atom, the isotopes may be present essentially in ratios according to their natural occurrence, or one or more particular atoms may be enhanced with respect to one or more isotopes using synthetic methods known to one skilled in the art. Thus, hydrogen includes for example ¹H, ²H, ³H; carbon includes for example ¹¹C, ¹²C, ¹³C, ¹⁴C; oxygen includes for example ¹⁶O, ¹⁷O, ¹⁸O; nitrogen includes for example ¹³N, ¹⁴N, ¹⁵N; sulfur includes for example ³²S, ³³S, ³⁴S, ³⁵S, ³⁶S, ³⁷S, ³⁸S; fluoro includes for example ¹⁷F, ¹⁸F, ¹⁹F; chloro includes for example ³⁵Cl, ³⁶Cl, ³⁷Cl, ³⁸Cl, ³⁹Cl; and the like.

As used herein, the terms “treat,” “treating,” “therapy,” “therapies,” and like terms refer to the administration of material, e.g., any one or more solid, crystalline or polymorphs of Compound I as described herein in an amount effective to prevent, alleviate, or ameliorate one or more symptoms of a disease or condition, i.e., indication, and/or to prolong the survival of the subject being treated.

The term “administering” refers to oral administration, administration as a suppository, topical contact, intravenous, intraperitoneal, intramuscular, intralesional, intranasal or subcutaneous administration, or the implantation of a slow-release device e.g., a mini-osmotic pump, to a subject. Administration is by any route, including parenteral and transmucosal (e.g., buccal, sublingual, palatal, gingival, nasal, vaginal, rectal, ortransdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

As used herein, the term “modulating” or “modulate” refers to an effect of altering a biological activity, especially a biological activity associated with a particular biomolecule such GLP-1. For example, an agonist or antagonist of a particular biomolecule modulates the activity of GLP-1 by either increasing (e.g. agonist, activator), or decreasing (e.g. antagonist, inhibitor) the activity, of the biomolecule. Such activity is typically indicated in terms of an inhibitory concentration (IC₅₀) or excitation concentration (EC₅₀) of the compound for an inhibitor or activator, respectively.

As used herein, the term “composition” refers to a pharmaceutical preparation suitable for administration to an intended subject for therapeutic purposes that contains at least one pharmaceutically active compound, including any solid form thereof. The composition may include at least one pharmaceutically acceptable component to provide an improved formulation of the compound, such as a suitable carrier or excipient.

As used herein, the term “subject” or “patient” refers to a living organism that is treated with compounds as described herein, including, but not limited to, any mammal, such as a human, other primates, sports animals, animals of commercial interest such as cattle, farm animals such as horses, or pets such as dogs and cats.

The term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile, e.g., for injectables.

In the present context, the term “therapeutically effective” or “effective amount” indicates that the materials or amount of material is effective to prevent, alleviate, or ameliorate one or more symptoms of a disease or medical condition, and/or to prolong the survival of the subject being treated. The therapeutically effective amount will vary depending on the compound, the disorder or condition and its severity and the age, weight, etc., of the mammal to be treated. For example, an effective amount is an amount sufficient to effectuate a beneficial or desired clinical result. The effective amounts can be provided all at once in a single administration or in fractional amounts that provide the effective amount in several administrations. The precise determination of what would be considered an effective amount may be based on factors individual to each subject, including their size, age, injury, and/or disease or injury being treated, and amount of time since the injury occurred or the disease began. One skilled in the art will be able to determine the effective amount for a given subject based on these considerations which are routine in the art.

In some embodiments, the phrase “substantially shown in Figure” as applied to an X-ray powder diffractogram is meant to include a variation of ±0.2°2θ or ±0.1°2θ, as applied to DSC thermograms is meant to include a variation of ±3° Celsius, and as applied to thermogravimetric analysis (TGA) is meant to include a variation of ±2% in weight loss.

“Substantially pure form (of a polymorph),” in some embodiments, means that in the referenced material, at least 99.9% of the material is the referenced polymorph. “Substantially pure form (of a polymorph),” in some embodiments, means that in the referenced material, at least 99.5% of the material is the referenced polymorph. “Substantially pure form (of a polymorph),” in some embodiments, means that in the referenced material, at least 99% of the material is the referenced polymorph. “Substantially pure form (of a polymorph),” in some embodiments, means that in the referenced material, at least 98% of the material is the referenced polymorph. “Substantially pure form (of a polymorph),” in some embodiments, means that in the referenced material, at least 97% of the material is the referenced polymorph. “Substantially pure form (of a polymorph),” in some embodiments, means that in the referenced material, at least 96% of the material is the referenced polymorph. “Substantially pure form (of a polymorph),” in some embodiments, means that in the referenced material, at least 95% of the material is the referenced polymorph. In the context of the use, testing, or screening of compounds that are or may be modulators, the term “contacting” means that the compound(s) are caused to be in sufficient proximity to a particular molecule, complex, cell, tissue, organism, or other specified material that potential binding interactions and/or chemical reaction between the compound and other specified material can occur.

### 2. Salts and Forms of Compound I

As described generally above, the present disclosure provides salts and crystalline forms of the compound, 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one (hereinafter “compound” of “Compound I”), and salts, co-crystals, solvates, or hydrates thereof. Crystalline forms of Compound I and salts, co-crystals, solvates, or hydrates thereof, and other forms (e.g., amorphous forms) of Compound I and salts, co-crystals, solvates, or hydrates thereof are collectively referred to herein as “forms of Compound I.”

In some embodiments, Compound I is in free form, e.g. a free acid. In some embodiments, Compound I is a salt. In some embodiments, Compound I is a pharmaceutically acceptable salt. In some embodiments, Compound I is a solvate. In some embodiments, Compound I is a hydrate. In some embodiments, Compound I is an anhydrate.

### Salts of Compound I

In one embodiment, provided is a salt of 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one (Compound I), or solvate thereof, having the formula IA:

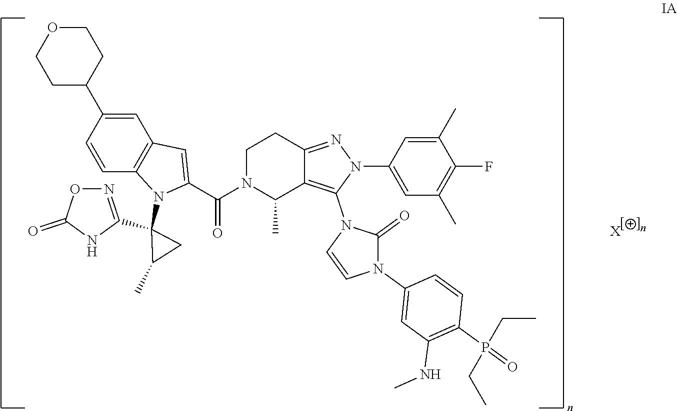

wherein: X is sodium and n is 1; or X is potassium and n is 1.

In one embodiment, provided is a 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one L-arginine salt (Compound I L-arginine salt), or solvate thereof, having the formula IB:

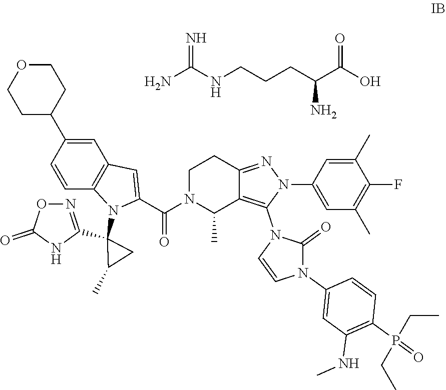 [SMILES: CCP(=O)(CC)c1ccc(-n2ccn(-c3c4c(nn3-c3cc(C)c(F)c(C)c3)CCN(C(=O)c3cc5cc(C6CCOCC6)ccc5n3[C@@]3(c5noc(=O)[nH]5)C[C@@H]3C)[C@H]4C)c2=O)cc1NC ; N=C(N)NCCC[C@H](N)C(=O)O]

In one embodiment is provided the L-arginine salt of Compound I or solvate thereof. In embodiment is provided the sodium salt of Compound I or solvate thereof. In embodiment is provided the potassium salt of Compound I or solvate thereof.

In one embodiment, provided is a crystalline salt form of 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one L-arginine salt (Compound I L-arginine salt), or solvate thereof.

In one embodiment, provided is crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one sodium salt (Compound I sodium salt), or solvate thereof. In one embodiment, provided is crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one potassium salt (Compound I potassium salt), or solvate thereof.

### Forms of Compound I

### Compound I L-Arginine Salt Form A

In one embodiment, provided is crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one L-arginine salt Form A (Compound I L-arginine salt Form A), characterized by an X-ray powder diffractogram comprising the following peaks expressed in ±0.2 degrees 2-theta selected from: 5.3, 9.1, and 11.5 as determined on a diffractometer using Cu-Kα radiation.

In some embodiments, Compound I L-arginine salt Form A is further characterized by an X-ray powder diffractogram comprising one or more additional peaks expressed in ±0.2 degrees 2-theta selected from: 13.8, 15.9, 16.5, 18.9, 20.9, and 22.8 as determined on a diffractometer using Cu-Kα radiation.

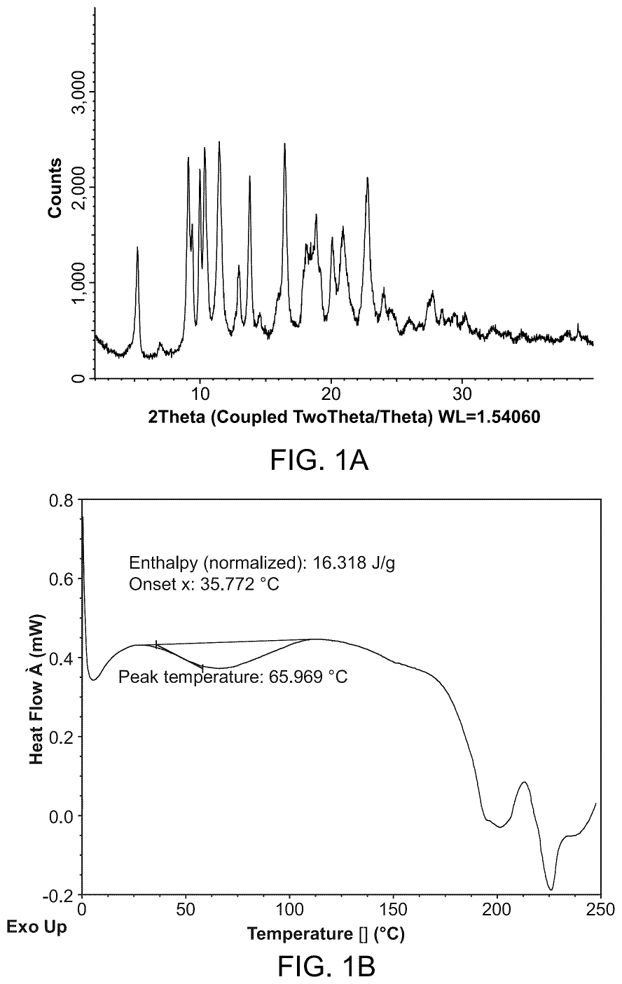

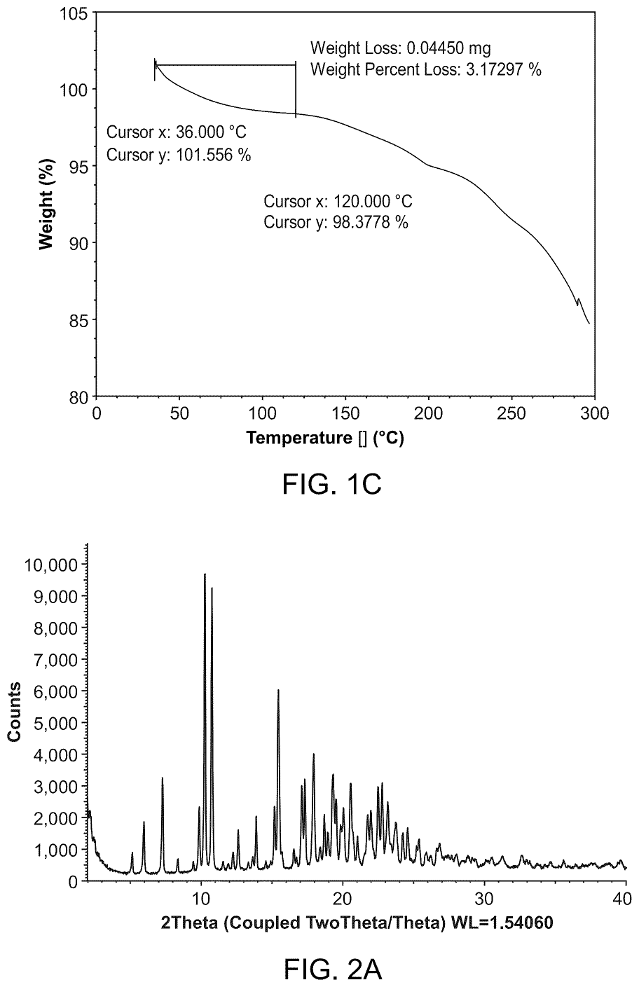

In some embodiments, Compound I L-arginine salt Form A is further characterized by an X-ray powder diffractogram as substantially shown in FIG. 1A.

In some embodiments, Compound I L-arginine salt Form A is further characterized by a DSC comprising an endotherm at about 66.0° C. (peak) and at about 35.8° C. (onset). In some embodiments, Compound I L-arginine salt Form A is further characterized by a DSC as substantially shown in FIG. 1B.

In some embodiments, crystalline Compound I L-arginine salt Form A is prepared via slurry of Compound I free acid Form A and equimolar L-arginine in THF at room temperature for 4 days.

In some embodiments, Compound I L-arginine salt Form A is further characterized by TGA showing a weight loss of about 3.2% up to about 120° C.

In some embodiments, the molar ratio of L-arginine/Compound I free acid in Compound I L-arginine salt Form A is about 1.0. In some embodiments, Compound I L-arginine salt Form A is a solvate. In some embodiments, Compound I L-arginine salt Form A is a THF solvate. In some embodiments, Compound I L-arginine salt Form A is a THF-water hetero solvate. In some embodiments, the molar ratio of THF/Compound I in Compound I L-arginine salt Form A is 0.8 (5.0 wt %). In some embodiments, the molar ratio of water/Compound I in Compound I L-arginine salt Form A is 1.0 (1.9 wt %).

### Compound I L-Arginine Salt Form B

In one embodiment, provided is crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one L-arginine salt Form B (Compound I L-arginine salt Form B), characterized by an X-ray powder diffractogram comprising the following peaks expressed in ±0.2 degrees 2-theta selected from: 6.1, 7.4, and 10.3, as determined on a diffractometer using Cu-Kα radiation.

In some embodiments, Compound I L-arginine salt Form B is further characterized by an X-ray powder diffractogram comprising one or more additional peaks expressed in ±0.2 degrees 2-theta selected from: 10.8, 15.3, 15.5, 18.0, 20.6, and 22.8 as determined on a diffractometer using Cu-Kα radiation.

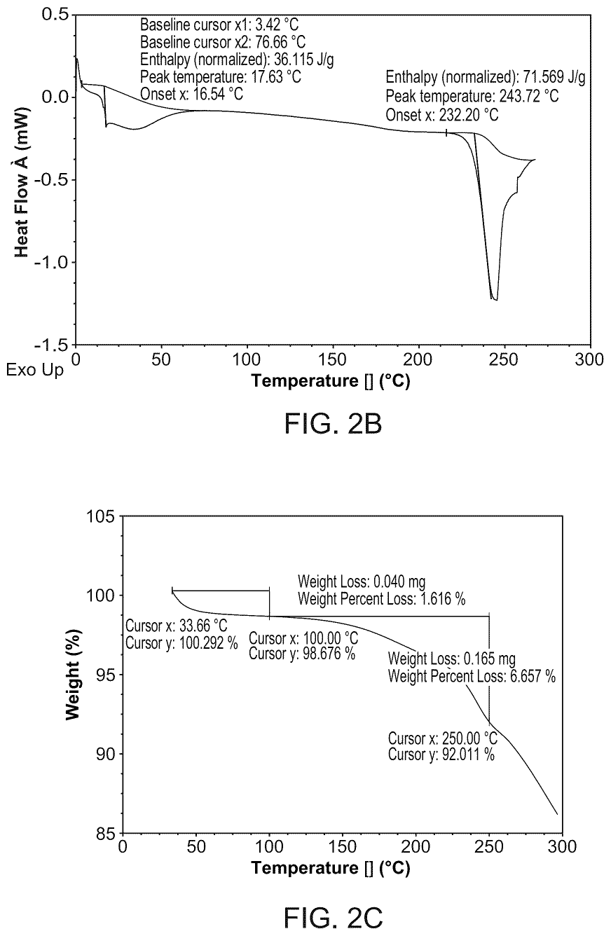

In some embodiments, Compound I L-arginine salt Form B is further characterized by an X-ray powder diffractogram as substantially shown in FIG. 2A.

In some embodiments, Compound I L-arginine salt Form B is further characterized by a DSC comprising an endotherm at about 17.6° C. (peak) and about 243.7° C. (peak). In some embodiments, Compound I L-arginine salt Form B is further characterized by a DSC as substantially shown in FIG. 2B.

In some embodiments, crystalline Compound I L-arginine salt Form B is prepared via equilibration of Compound I L-arginine salt Form A in IPA/water (17:1 v/v) at room temperature for about 2 weeks. In some embodiments, crystalline Compound I L-arginine salt Form B is prepared via equilibration of Compound I L-arginine salt Form A IPA/water (17:1 v/v) at 50° C. for 1 week.

In some embodiments, Compound I L-arginine salt Form B is further characterized by TGA showing a two-step weight loss of about 1.6% up to about 100° C. and about 6.7% from about 100-250° C.

In some embodiments, the molar ratio of L-arginine/Compound I free acid in Compound I L-arginine salt Form B is about 1.0. In some embodiments, Compound I L-arginine salt Form B is a solvate. In some embodiments, Compound I L-arginine salt Form B is an IPA solvate. In some embodiments, Compound I L-arginine salt Form B is a IPA-water hetero solvate. In some embodiments, the molar ratio of IPA/Compound I in Compound I L-arginine salt Form B is 0.8 (6.0 wt %). In some embodiments, the molar ratio of water/Compound I in Compound I L-arginine salt Form B is 1.7 (2.6 wt %).

In some embodiments, Compound I L-arginine salt Form B is a hydrate. In some embodiments, Compound I L-arginine salt Form B is a channel hydrate.

It was found that Compound I L-arginine salt Form B exhibited enhanced pharmacokinetic properties, e.g., better exposure, than other forms.

### Compound I L-Arginine Salt Form C

In one embodiment, provided is crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one L-arginine salt Form C (Compound I L-arginine salt Form C), characterized by an X-ray powder diffractogram comprising the following peaks expressed in ±0.2 degrees 2-theta selected from: 6.1, 7.4, and 10.3 as determined on a diffractometer using Cu-Kα radiation. Form C is the hydrate of form B described above.

In some embodiments, Compound I L-arginine salt Form C, e.g., the hydrate of form B, is further characterized by an X-ray powder diffractogram comprising one or more additional peaks expressed in ±0.2 degrees 2-theta selected from: 10.8, 15.3, 15.5, 18.0, 20.6, and 22.8 as determined on a diffractometer using Cu-Kα radiation.

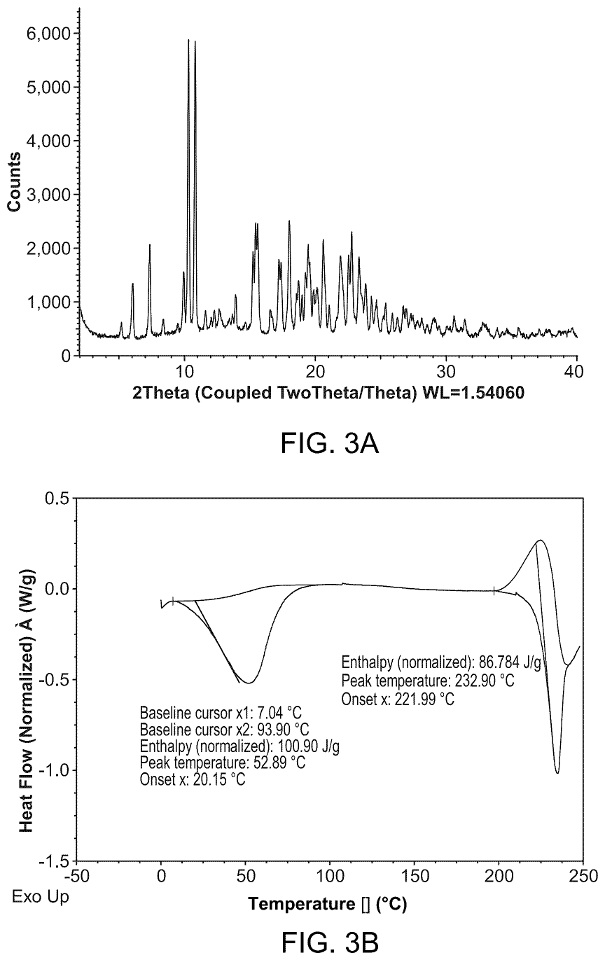

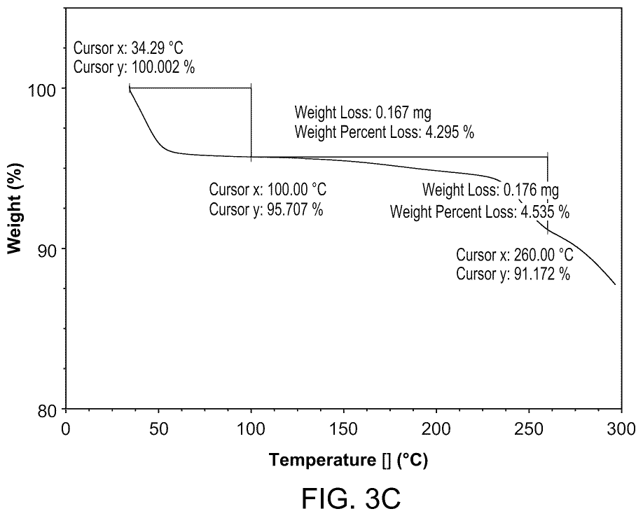

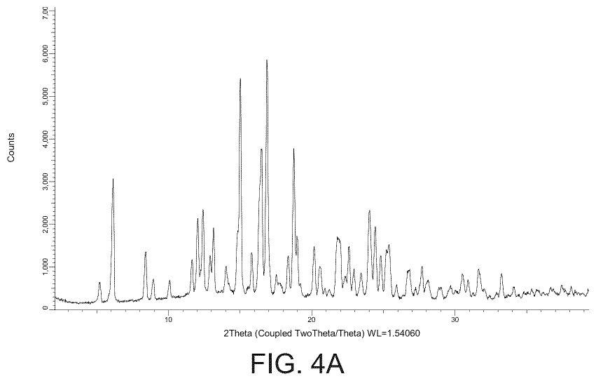

In some embodiments, Compound I L-arginine salt Form C is further characterized by an X-ray powder diffractogram as substantially shown in FIG. 3A.

In some embodiments, Compound I L-arginine salt Form C is further characterized by a DSC comprising two endotherms at about 52.9° C. (peak) and about 232.9° C. (peak). In some embodiments, Compound I L-arginine salt Form C is further characterized by a DSC as substantially shown in FIG. 3B.

In some embodiments, crystalline Compound I L-arginine salt Form C is prepared by equilibration of Compound I L-arginine salt Form A in acetone at room temperature for 2 weeks. In some embodiments, crystalline Compound I L-arginine salt Form C is prepared by equilibration of Compound I L-arginine salt Form A in ACN at room temperature for 2 weeks. In some embodiments, crystalline Compound I L-arginine salt Form C is prepared by equilibration of Compound I L-arginine salt Form A in ethanol at 50° C. for 1 week. In some embodiments, crystalline Compound I L-arginine salt Form C is prepared by equilibration of Compound I L-arginine salt Form A in acetone at 50° C. for 1 week. In some embodiments, crystalline Compound I L-arginine salt Form C is prepared by equilibration of Compound I L-arginine salt Form A in ACN at 50° C. for 1 week. In some embodiments, crystalline Compound I L-arginine salt Form C is prepared by equilibration of Compound I L-arginine salt Form A in ACN/water (1:1 v/v) at 50° C. for 1 week.

In some embodiments, Compound I L-arginine salt Form C is further characterized by TGA showing a two-step weight loss of about 4.2% up to about 100° C. and about 4.5% from about 100-260° C.

In some embodiments, the molar ratio of L-arginine/Compound I free acid in Compound I L-arginine salt Form C is about 1.0. In some embodiments, Compound I L-arginine salt Form C is a hydrate. In some embodiments, the molar ratio of acetone/Compound I in Compound I L-arginine salt Form C is 0.02 (0.1 wt %). In some embodiments, the molar ratio of water/Compound I in Compound I L-arginine salt Form C is 4.4 (6.8 wt %).

An initial polymorph screen failed to provide a suitable substantially crystalline form of Compound I. Subsequent polymorph screens are described below.

### Compound I Free Acid Form A

In one embodiment, provided is 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one free acid Form A (Compound I free acid Form A), characterized by an X-ray powder diffractogram comprising the following peaks expressed in ±0.2 degrees 2-theta selected from: 5.2, 6.1, and 12.4, as determined on a diffractometer using Cu-Kα radiation.

In some embodiments, Compound I free acid Form A is further characterized by an X-ray powder diffractogram comprising one or more additional peaks expressed in ±0.2 degrees 2-theta selected from: 15.0, 16.5, 16.9, 18.8, 20.2, and 21.9, as determined on a diffractometer using Cu-Kα radiation.

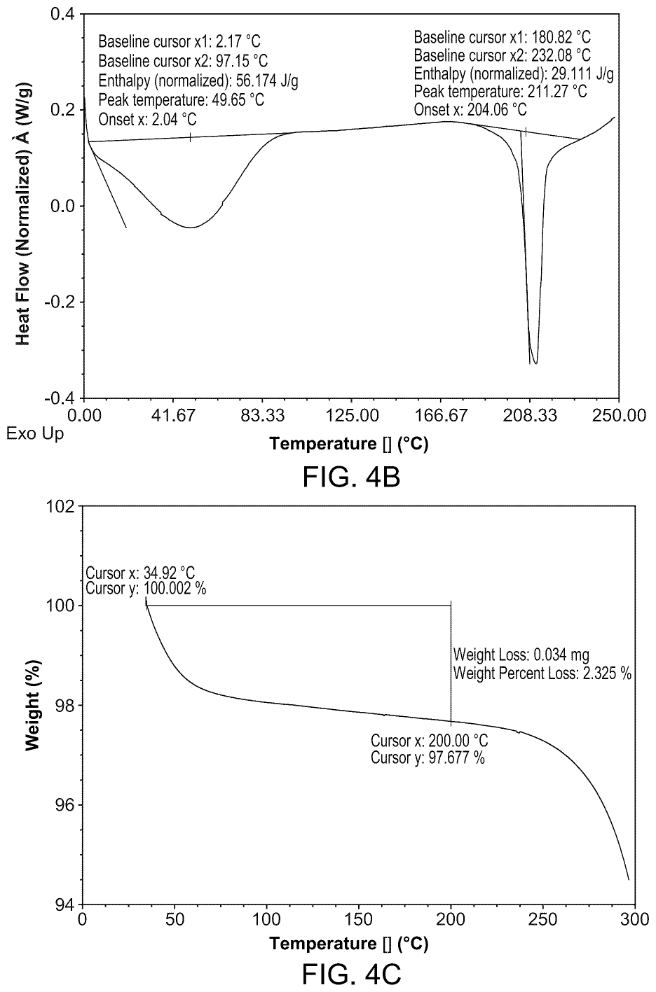

In some embodiments, Compound I free acid Form A is further characterized by an X-ray powder diffractogram as substantially shown in FIG. 4A.

In some embodiments, Compound I free acid Form A is further characterized by a DSC comprising two endotherms at about 49.7° C. (peak) and about 211.3° C. (peak). In some embodiments, crystalline Compound I free acid Form A is further characterized by a DSC as substantially shown in FIG. 4B.

In some embodiments, Compound I free acid Form A is obtained via slurry of Compound I free acid in EtOAc at room temperature for 2 days.

In some embodiments, Compound I free acid Form A is further characterized by TGA showing a weight loss of about 2.3% up to about 200° C.

In some embodiments, Compound I free acid Form A is a hydrate. In some embodiments, the molar ratio of water/Compound I free acid in Compound I free acid Form A is 1.7 (3.3 wt %).

### Compound I Free Acid Form B

In one embodiment, provided is 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one free acid Form B (Compound I free acid Form B), characterized by an X-ray powder diffractogram comprising the following peaks expressed in ±0.2 degrees 2-theta selected from: 7.8, 9.2, and 10.0, as determined on a diffractometer using Cu-Kα radiation.

In some embodiments, Compound I free acid Form B is further characterized by an X-ray powder diffractogram comprising one or more additional peaks expressed in ±0.2 degrees 2-theta selected from: 10.3, 13.0, 13.7, 16.5, 20.5, and 23.2, as determined on a diffractometer using Cu-Kα radiation.

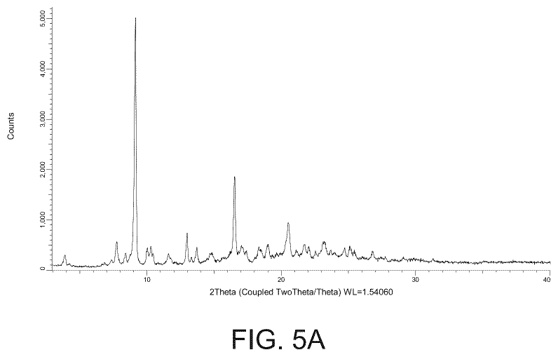

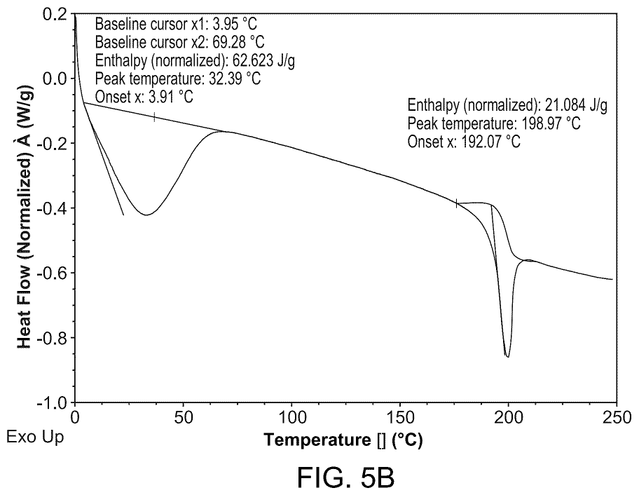

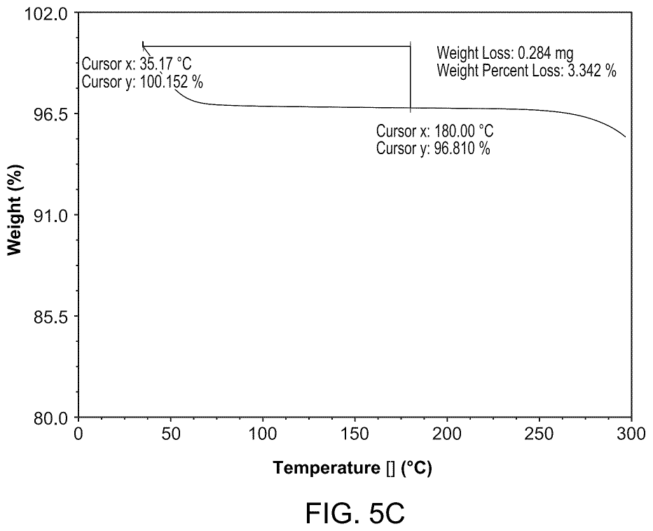

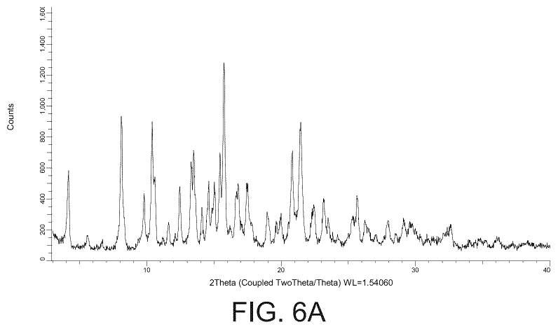

In some embodiments, Compound I free acid Form B is further characterized by an X-ray powder diffractogram as substantially shown in FIG. 5A.

In some embodiments, Compound I free acid Form B is further characterized by a DSC comprising two endotherms at about 32.4° C. (peak) and about 199.0° C. (peak). In some embodiments, crystalline Compound I free acid Form B is further characterized by a DSC as substantially shown in FIG. 5B.

In some embodiments, Compound I free acid Form B is obtained via equilibration of Compound I free acid Form A in ACN under a temperature cycle between 5-50° C. at a heating/cooling rate of 0.1° C./min for 10 cycles. In some embodiments, Compound I free acid Form B is obtained via crystallization by slow cooling from a saturated solution of Compound I free acid Form A in ACN at 50° C. cooled at 0.1° C./min to 5° C.

In some embodiments, Compound I free acid Form B is further characterized by TGA showing a weight loss of about 3.3% up to about 180° C.

In some embodiments, Compound I free acid Form B is a hydrate. In some embodiments, the molar ratio of water/Compound I free acid in Compound I free acid Form B is 2.5 (4.6 wt %).

### Compound I Free Acid Form C

In one embodiment, provided is 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one free acid Form C (Compound I free acid Form C), characterized by an X-ray powder diffractogram comprising the following peaks expressed in ±0.2 degrees 2-theta selected from: 4.1, 8.1, and 10.4, as determined on a diffractometer using Cu-Kα radiation.

In some embodiments, Compound I free acid Form C is further characterized by an X-ray powder diffractogram comprising one or more additional peaks expressed in ±0.2 degrees 2-theta selected from: 13.5, 14.6, 15.0, 15.5, 15.8, and 20.8, as determined on a diffractometer using Cu-Kα radiation.

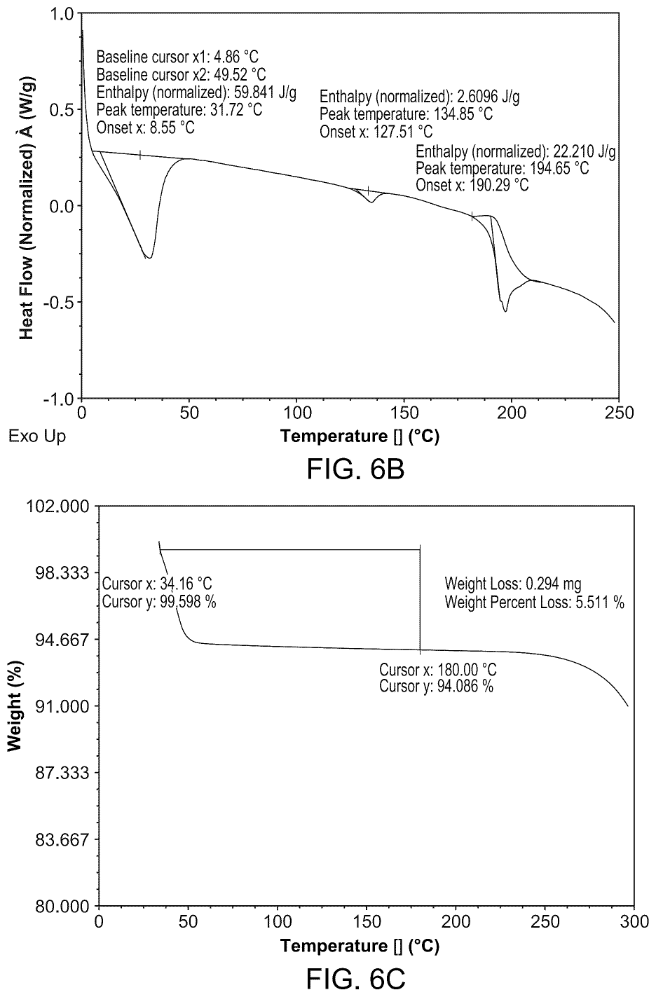

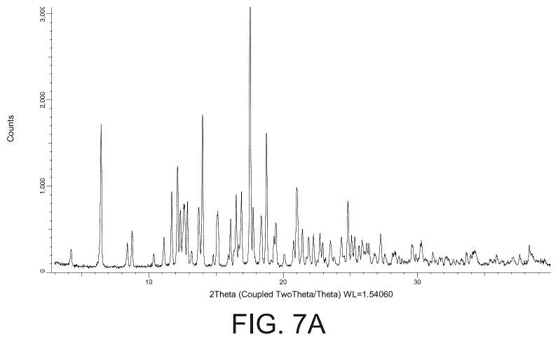

In some embodiments, Compound I free acid Form C is further characterized by an X-ray powder diffractogram as substantially shown in FIG. 6A.

In some embodiments, Compound I free acid Form C is further characterized by a DSC comprising three endotherms at about 31.7° C. (peak), about 134.9° C. (peak), and about 194.7° C. (peak). In some embodiments, crystalline Compound I free acid Form C is further characterized by a DSC as substantially shown in FIG. 6B.

In some embodiments, Compound I free acid Form C is obtained via equilibration of Compound I free acid Form A in MTBE at room temperature for 2 weeks. In some embodiments, Compound I free acid Form C is obtained via equilibration of Compound I free acid Form A in MTBE at 50° C. for 1 week. In some embodiments, Compound I free acid Form C is obtained via equilibration of Compound I free acid Form A in MTBE under a temperature cycle between 5-50° C. at a heating/cooling rate of 0.1° C./min for 10 cycles.

In some embodiments, Compound I free acid Form C is further characterized by TGA showing a weight loss of about 5.5% up to about 180° C.

In some embodiments, Compound I free acid C is a solvate. In some embodiments, the molar ratio of MTBE/Compound I free acid in Compound I free acid Form C is 0.02 (0.2 wt %). In some embodiments, the molar ratio of water/Compound I free acid in Compound I free acid Form C is 2.7 (5.1 wt %).

### Compound I Free Acid Form D

In one embodiment, provided is 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one free acid Form D (Compound I free acid Form D), characterized by an X-ray powder diffractogram comprising the following peaks expressed in ±0.2 degrees 2-theta selected from: 6.5, 12.1, and 12.9, as determined on a diffractometer using Cu-Kα radiation.

In some embodiments, Compound I free acid Form D is further characterized by an X-ray powder diffractogram comprising one or more additional peaks expressed in ±0.2 degrees 2-theta selected from: 14.0, 16.5, 16.9, 17.5, 18.8, and 21.0, as determined on a diffractometer using Cu-Kα radiation.

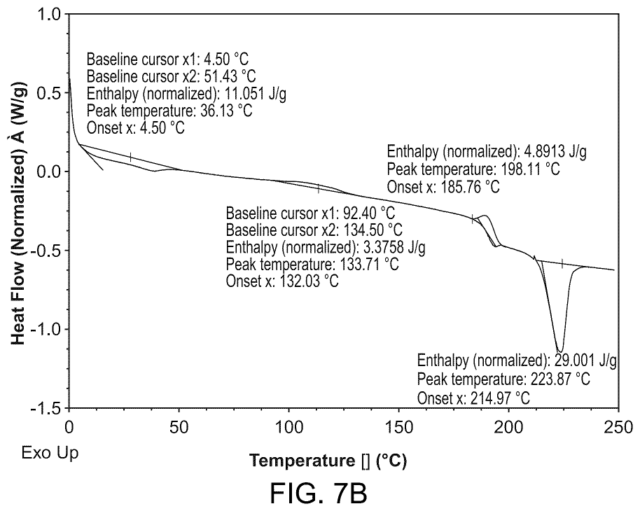

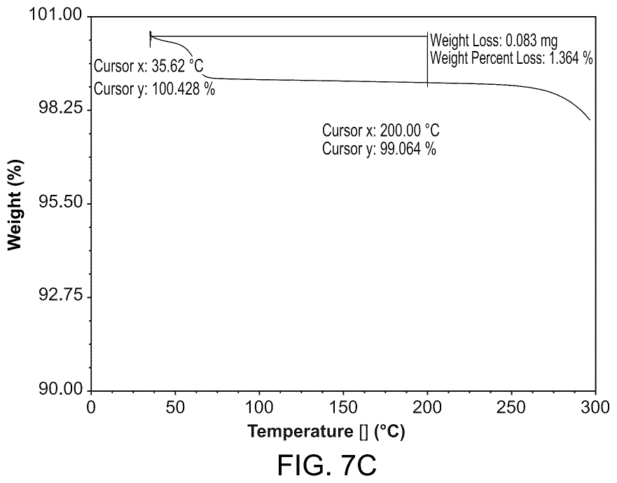

In some embodiments, Compound I free acid Form D is further characterized by an X-ray powder diffractogram as substantially shown in FIG. 7A.

In some embodiments, Compound I free acid Form D is further characterized by a DSC comprising three endotherms at about 36.1° C. (peak), about 198.1° C. (peak), and about 223.9° C. (peak) and a broad exotherm at about 133.7° C. (peak). In some embodiments, crystalline Compound I free acid Form D is further characterized by a DSC as substantially shown in FIG. 7B.

In some embodiments, Compound I free acid Form D is obtained via equilibration of Compound I free acid Form A in ACN/water (9:1 v/v) at room temperature for 2 weeks. In some embodiments, Compound I free acid Form D is obtained via equilibration of Compound I free acid Form A in ACN/water (9:1 v/v) under a temperature cycle between 5-50° C. at a heating/cooling rate of 0.1° C./min for 10 cycles.

In some embodiments, Compound I free acid Form D is further characterized by TGA showing a weight loss of about 1.4% up to about 200° C.

In some embodiments, Compound I free acid Form D is a hydrate. In some embodiments, the molar ratio of water/Compound I free acid in Compound I free acid Form D is 1.1 (2.2 wt %).

### Compound I Free Acid Form E

In one embodiment, provided is 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one free acid Form E (Compound I free acid Form E), characterized by an X-ray powder diffractogram comprising the following peaks expressed in ±0.2 degrees 2-theta selected from: 5.7, 11.1, and 16.1, as determined on a diffractometer using Cu-Kα radiation.

In some embodiments, Compound I free acid Form E is further characterized by an X-ray powder diffractogram comprising one or more additional peaks expressed in ±0.2 degrees 2-theta selected from: 17.1, 18.1, 18.7, 21.0, 21.3, and 21.6, as determined on a diffractometer using Cu-Kα radiation.

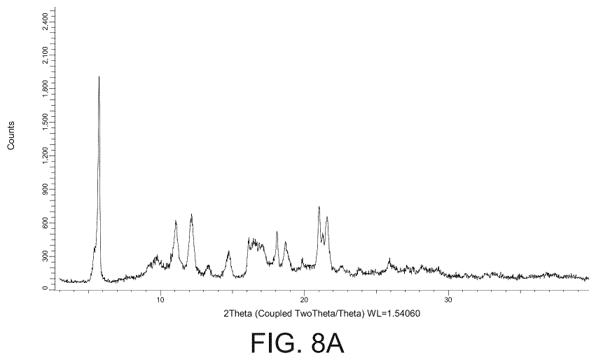

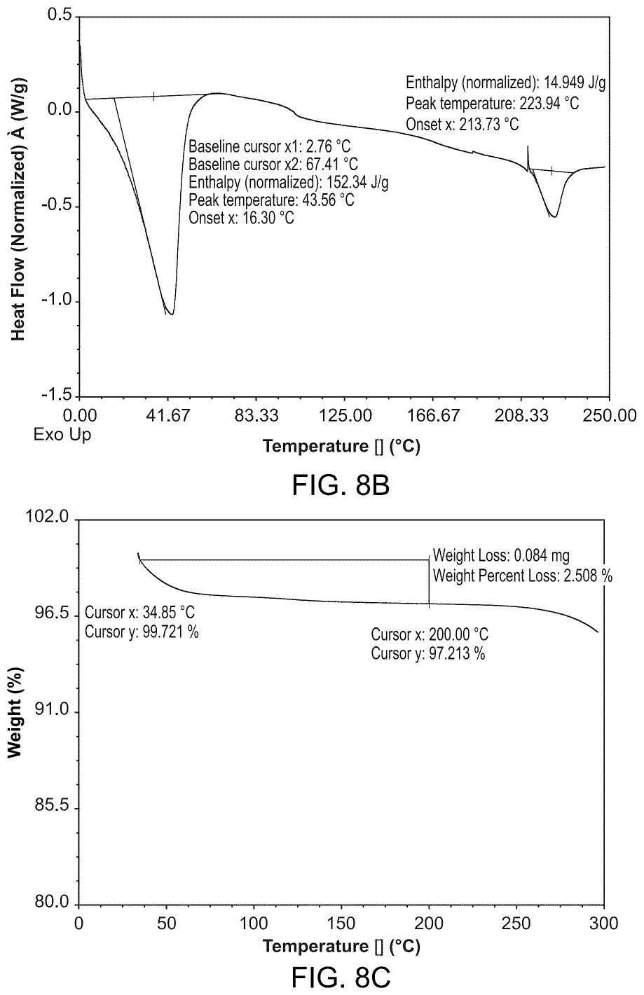

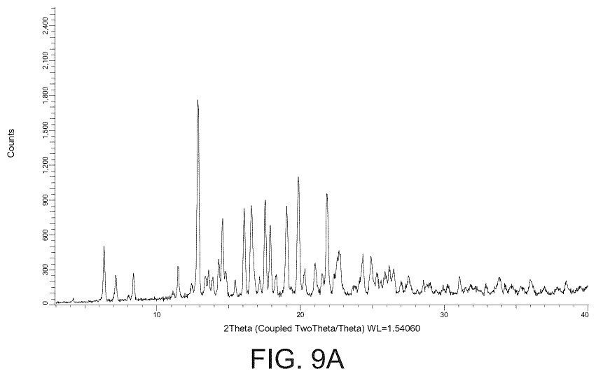

In some embodiments, Compound I free acid Form E is further characterized by an X-ray powder diffractogram as substantially shown in FIG. 8A.

In some embodiments, Compound I free acid Form E is further characterized by a DSC comprising two endotherms at about 43.6° C. (peak) and about 223.9° C. (peak). In some embodiments, crystalline Compound I free acid Form E is further characterized by a DSC as substantially shown in FIG. 8B.

In some embodiments, Compound I free acid Form E is obtained via equilibration of Compound I free acid Form A in THF/water (9:1 v/v) at room temperature for 2 weeks. In some embodiments, Compound I free acid Form E is obtained via equilibration of Compound I free acid Form A in THF/water (9:1 v/v) at 50° C. for 1 week. In some embodiments, Compound I free acid Form E is obtained via equilibration of Compound I free acid Form A in THF/water (9:1 v/v) under a temperature cycle between 5-50° C. at a heating/cooling rate of 0.1° C./min for 10 cycles. In some embodiments, Compound I free acid Form E is obtained via crystallization of Compound I free acid Form A in 1,4-dioxane by addition of anti-solvent (water).

In some embodiments, Compound I free acid Form E is further characterized by TGA showing a weight loss of about 2.5% up to about 200° C.

In some embodiments, Compound I free acid Form E is a hydrate. In some embodiments, the molar ratio of water/Compound I free acid in Compound I free acid Form E is 5.8 (10.3 wt %).

### Compound I Free Acid Form F

In one embodiment, provided is 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one free acid Form F (Compound I free acid Form F), characterized by an X-ray powder diffractogram comprising the following peaks expressed in ±0.2 degrees 2-theta selected from: 7.2, 12.9, and 14.6, as determined on a diffractometer using Cu-Kα radiation.

In some embodiments, Compound I free acid Form F is further characterized by an X-ray powder diffractogram comprising one or more additional peaks expressed in ±0.2 degrees 2-theta selected from: 16.1, 16.6, 17.5, 19.1, 19.9, and 21.8, as determined on a diffractometer using Cu-Kα radiation.

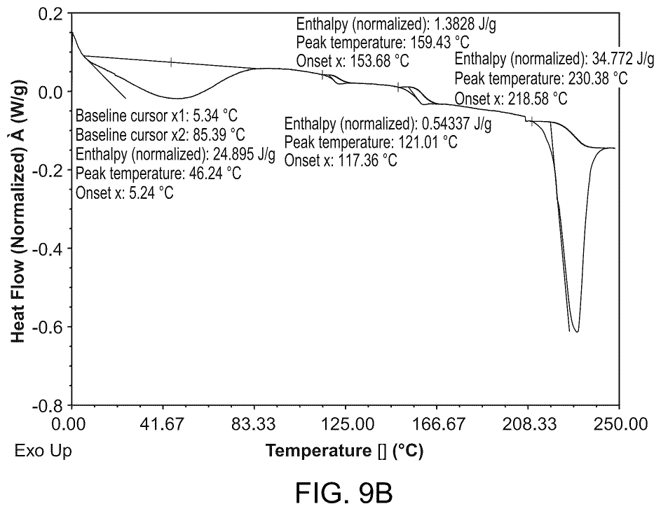

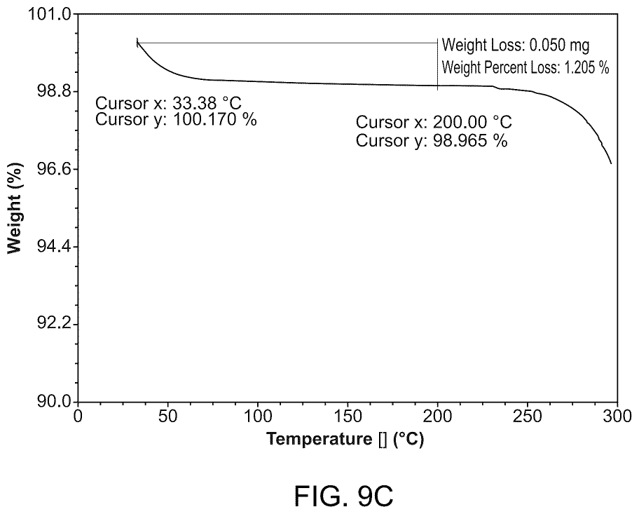

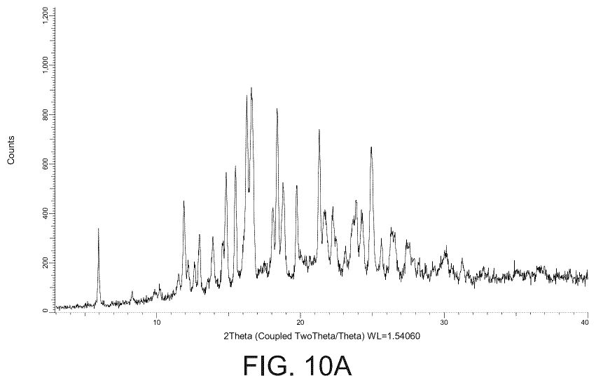

In some embodiments, Compound I free acid Form F is further characterized by an X-ray powder diffractogram as substantially shown in FIG. 9A.

In some embodiments, Compound I free acid Form F is further characterized by a DSC comprising endotherms at about 46.2° C. (peak), about 121.0° C. (peak), about 159.4° C. (peak), and about 230.4° C. (peak). In some embodiments, crystalline Compound I free acid Form F is further characterized by a DSC as substantially shown in FIG. 9B.

In some embodiments, Compound I free acid Form F is obtained via equilibration of Compound I free acid Form A in ACN at room temperature for 2 weeks. In some embodiments, Compound I free acid Form E is obtained via equilibration of Compound I free acid Form F in ACN at 50° C. for 1 week. In some embodiments, Compound I free acid Form F is obtained via crystallization by rapid cooling of a saturated solution of Compound I free acid Form A in ACN at 50° C. cooled rapidly to 5° C.

In some embodiments, Compound I free acid Form F is further characterized by TGA showing a weight loss of about 1.2% up to about 200° C.

In some embodiments, Compound I free acid Form F is a hydrate. In some embodiments, the molar ratio of water/Compound I free acid in Compound I free acid Form F is 0.9 (1.7 wt %).

### Compound I Free Acid Form G

In one embodiment, provided is 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one free acid Form G (Compound I free acid Form G), characterized by an X-ray powder diffractogram comprising the following peaks expressed in ±0.2 degrees 2-theta selected from: 6.0, 11.9, and 14.8, as determined on a diffractometer using Cu-Kα radiation.

In some embodiments, Compound I free acid Form G is further characterized by an X-ray powder diffractogram comprising one or more additional peaks expressed in ±0.2 degrees 2-theta selected from: 16.3, 16.6, 18.4, 18.8, 21.3, and 23.9, as determined on a diffractometer using Cu-Kα radiation.

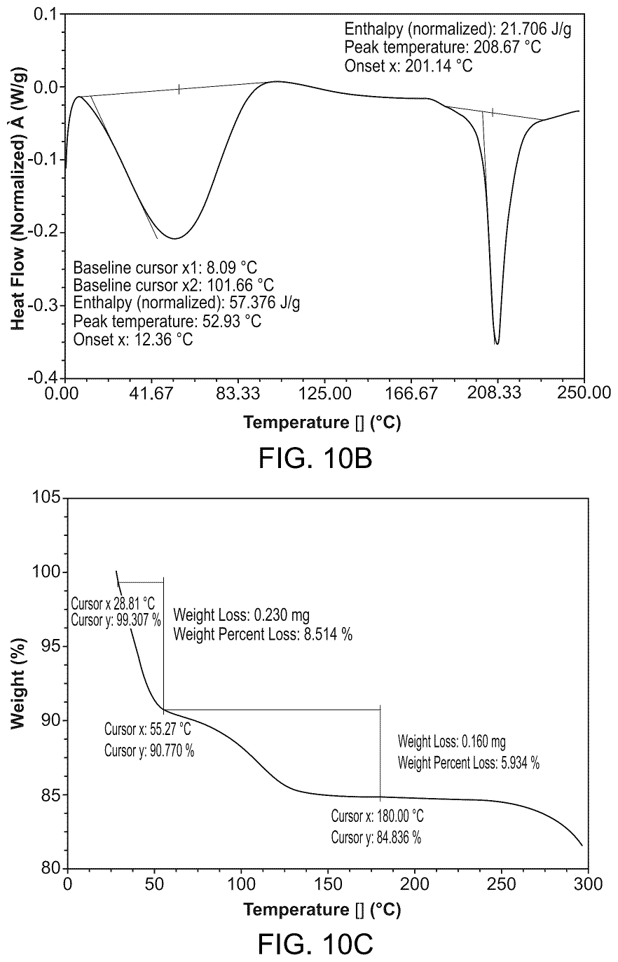

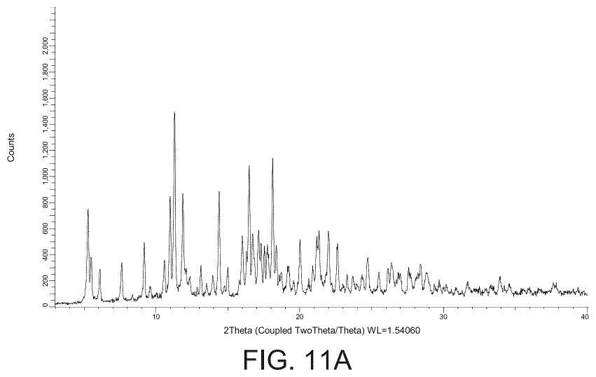

In some embodiments, Compound I free acid Form G is further characterized by an X-ray powder diffractogram as substantially shown in FIG. 10A.

In some embodiments, Compound I free acid Form G is further characterized by a DSC comprising two endotherms at about 52.9° C. (peak) and about 208.7° C. (peak). In some embodiments, crystalline Compound I free acid Form G is further characterized by a DSC as substantially shown in FIG. 10B.

In some embodiments, Compound I free acid Form G is obtained via equilibration of Compound I free acid Form A in DMSO/water (1:1 v/v) at room temperature for 2 weeks. In some embodiments, Compound I free acid Form G is obtained via equilibration of Compound I free acid Form A in DMSO/water (1:1 v/v) at 50° C. for 1 week.

In some embodiments, Compound I free acid Form G is further characterized by TGA showing a two-step weight loss of about 8.5% up to about 55° C. and about 5.9% from about 55-180° C.

In some embodiments, Compound I free acid Form G is a hydrate. In some embodiments, the molar ratio of water/Compound I free acid in Compound I free acid Form G is 8.4 (14.1 wt %). In some embodiments, the molar ratio of DMSO/Compound I free acid in Compound I free acid Form G is 0.1 (0.7 wt %).

### Compound I Free Acid Form H

In one embodiment, provided is 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one free acid Form H (Compound I free acid Form H), characterized by an X-ray powder diffractogram comprising the following peaks expressed in ±0.2 degrees 2-theta selected from: 5.3, 5.5, and 7.6, as determined on a diffractometer using Cu-Kα radiation.

In some embodiments, Compound I free acid Form H is further characterized by an X-ray powder diffractogram comprising one or more additional peaks expressed in ±0.2 degrees 2-theta selected from: 11.0, 11.3, 11.9, 14.4, 16.5, and 18.1, as determined on a diffractometer using Cu-Kα radiation.

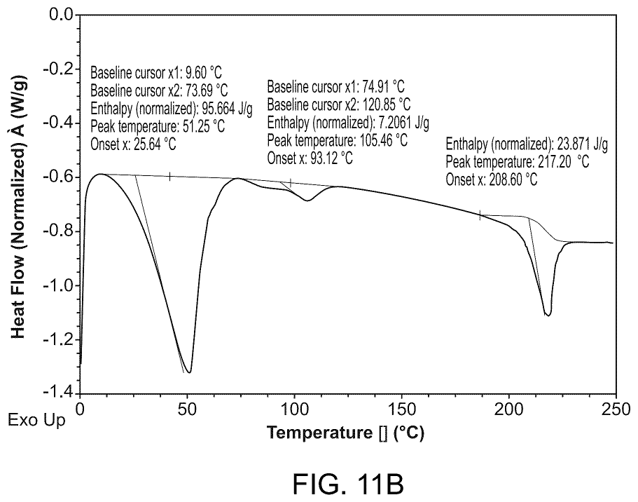

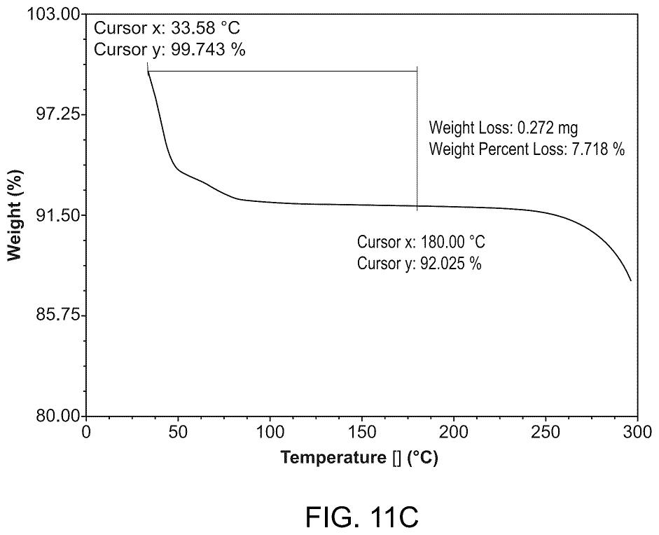

In some embodiments, Compound I free acid Form H is further characterized by an X-ray powder diffractogram as substantially shown in FIG. 11A.

In some embodiments, Compound I free acid Form H is further characterized by a DSC comprising endotherms at about 51.3° C. (peak), about 105.5° C. (peak), and about 217.2° C. (peak). In some embodiments, crystalline Compound I free acid Form H is further characterized by a DSC as substantially shown in FIG. 11B.

In some embodiments, Compound I free acid Form H is obtained via equilibration of Compound I free acid Form A in acetone/water (1:1 v/v) at room temperature for 2 weeks. In some embodiments, Compound I free acid Form H is obtained via addition of anti-solvent (water) to Compound I free acid Form A in 1,4-dioxane, followed by equilibration for 10 days.

In some embodiments, Compound I free acid Form H is further characterized by TGA showing a weight loss of about 7.7% up to about 180° C.

In some embodiments, Compound I free acid Form H is a hydrate. In some embodiments, the molar ratio of water/Compound I free acid in Compound I free acid Form H is 5.5 (9.6 wt %).

### Compound I Free Acid Form I

In one embodiment, provided is 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one free acid Form I (Compound I free acid Form I), characterized by an X-ray powder diffractogram comprising the following peaks expressed in ±0.2 degrees 2-theta selected from: 4.0, 12.2, and 14.0, as determined on a diffractometer using Cu-Kα radiation.

In some embodiments, Compound I free acid Form I is further characterized by an X-ray powder diffractogram comprising one or more additional peaks expressed in ±0.2 degrees 2-theta selected from: 15.1, 15.8, 16.7, 18.4, 21.0, and 22.0, as determined on a diffractometer using Cu-Kα radiation.

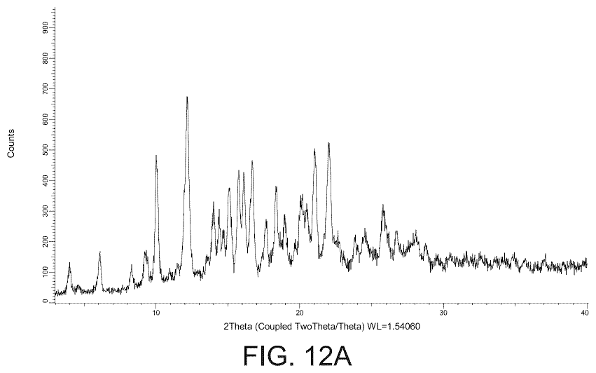

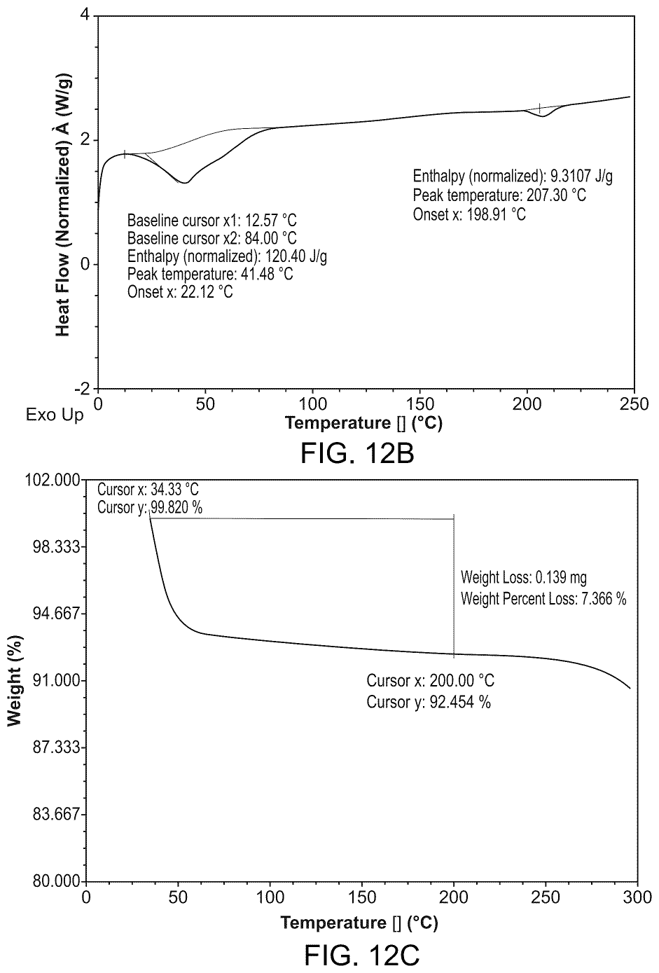

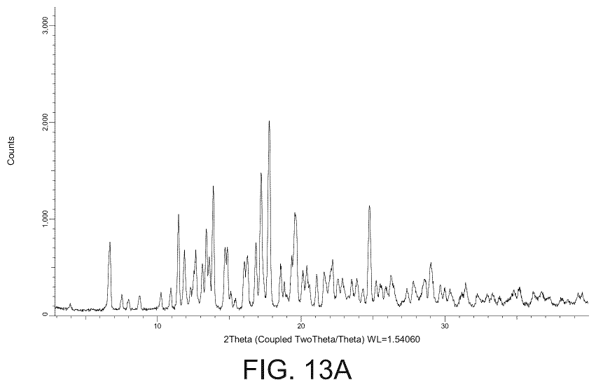

In some embodiments, Compound I free acid Form I is further characterized by an X-ray powder diffractogram as substantially shown in FIG. 12A.

In some embodiments, Compound I free acid Form I is further characterized by a DSC comprising endotherms at about 41.5° C. (peak) and about 207.3° C. (peak). In some embodiments, crystalline Compound I free acid Form I is further characterized by a DSC as substantially shown in FIG. 12B.

In some embodiments, Compound I free acid Form I is obtained via addition of Compound I free acid Form A in 1,4-dioxane into anti-solvent (water), followed by equilibration for 10 days.

In some embodiments, Compound I free acid Form I is further characterized by TGA showing a weight loss of about 7.4% up to about 200° C.

### Compound I Free Acid Form J

In one embodiment, provided is 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one free acid Form J (Compound I free acid Form J), characterized by an X-ray powder diffractogram comprising the following peaks expressed in ±0.2 degrees 2-theta selected from: 6.8, 11.6, and 13.5, as determined on a diffractometer using Cu-Kα radiation.

In some embodiments, Compound I free acid Form J is further characterized by an X-ray powder diffractogram comprising one or more additional peaks expressed in ±0.2 degrees 2-theta selected from: 14.0, 15.0, 17.0, 17.3, 17.9, and 19.7, as determined on a diffractometer using Cu-Kα radiation.

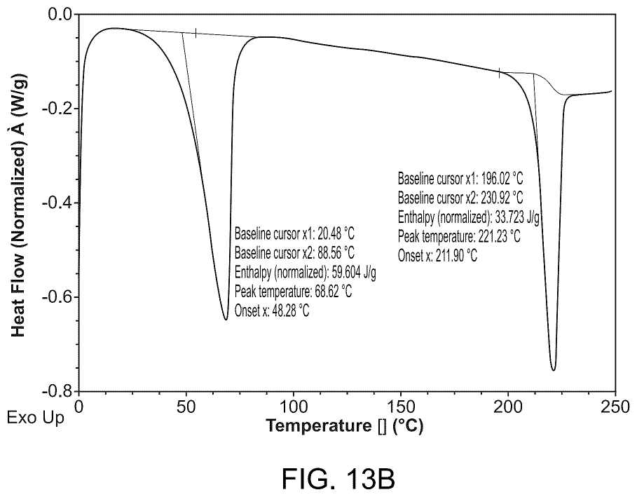

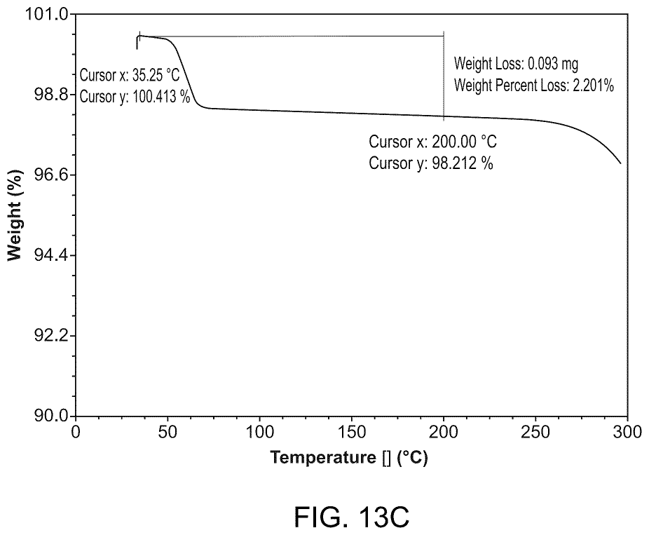

In some embodiments, Compound I free acid Form J is further characterized by an X-ray powder diffractogram as substantially shown in FIG. 13A.

In some embodiments, Compound I free acid Form J is further characterized by a DSC comprising endotherms about 68.6° C. (peak) and about 221.2° C. (peak). In some embodiments, crystalline Compound I free acid Form J is further characterized by a DSC as substantially shown in FIG. 13B.

In some embodiments, Compound I free acid Form J is obtained after storing Compound I free acid Form D at ambient conditions (23-27° C., 50-70% RH) for 2 weeks.

In some embodiments, Compound I free acid Form J is further characterized by TGA showing a weight loss of about 2.2% up to about 200° C.

### Compound I Free Acid Form K

In one embodiment, provided is 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one free acid Form K (Compound I free acid Form K), characterized by an X-ray powder diffractogram comprising the following peaks expressed in +0.2 degrees 2-theta selected from: 8.7, 9.9, and 12.5, as determined on a diffractometer using Cu-Kα radiation.

In some embodiments, Compound I free acid Form K is further characterized by an X-ray powder diffractogram comprising one or more additional peaks expressed in +0.2 degrees 2-theta selected from: 14.2, 15.8, 16.4, 19.6, 21.1, and 23.6, as determined on a diffractometer using Cu-Kα radiation.

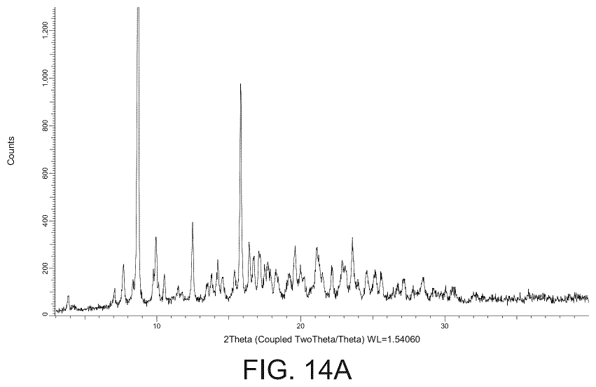

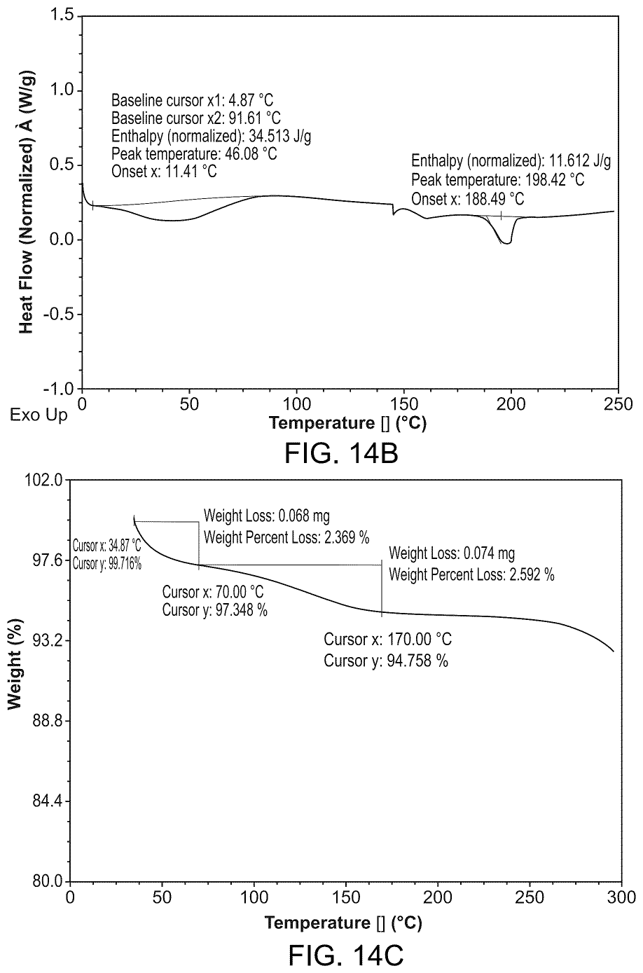

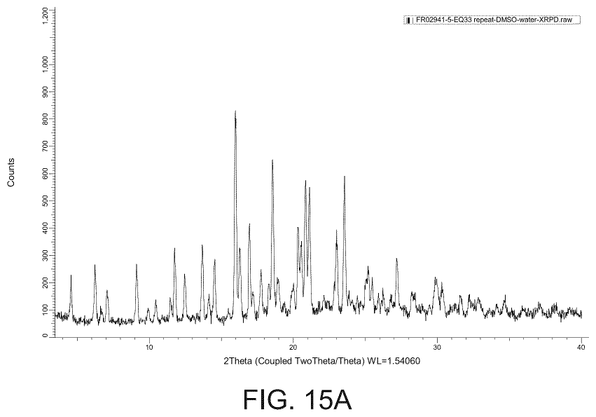

In some embodiments, Compound I free acid Form K is further characterized by an X-ray powder diffractogram as substantially shown in FIG. 14A.

In some embodiments, Compound I free acid Form K is further characterized by a DSC comprising endotherms at about 46.1° C. (peak) and about 198.4° C. (peak). In some embodiments, crystalline Compound I free acid Form K is further characterized by a DSC as substantially shown in FIG. 14B.

In some embodiments, Compound I free acid Form K is obtained after storing Compound I free acid Form B at ambient conditions (23-27° C., 50-70% RH) for 2 weeks.

In some embodiments, Compound I free acid Form K is further characterized by TGA showing a two-step weight loss of about 2.4% up to about 70° C. and about 2.6% from about 70-170° C.

### Compound I Free Acid Form L

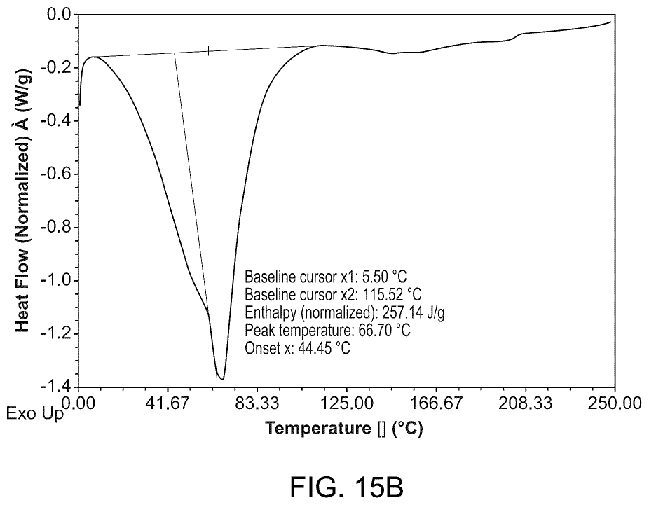

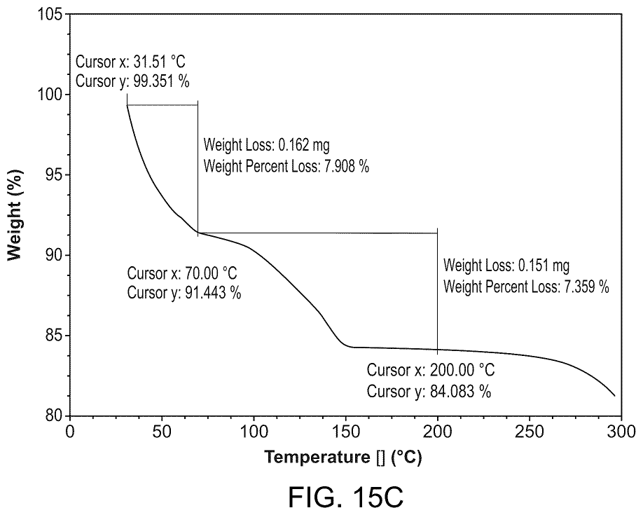

In one embodiment, provided is 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one free acid Form L (Compound I free acid Form L), characterized by an X-ray powder diffractogram as substantially shown in FIG. 15A. Form L is a DMSO-water hetero solvate.

In some embodiments, Compound I free acid Form L is further characterized by a DSC comprising an endotherm at about 120° C. (peak). In some embodiments, crystalline Compound I free acid Form L is further characterized by a DSC as substantially shown in FIG. 15B.

In some embodiments, Compound I free acid Form L shows about a about 7.9% weight loss at about 70° C. and about 7.4% weight loss from about 70° C. to about 200° C. In one embodiment, the form is characterized by a TGA plot as substantially shown in FIG. 15C.

### Compound I Free Acid Form M

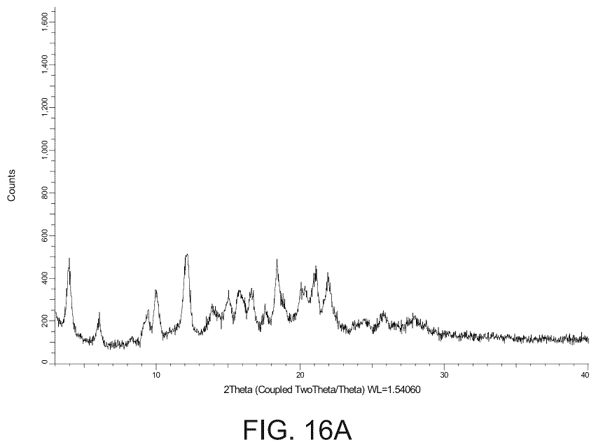

In one embodiment, provided is 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one free acid Form M (Compound I free acid Form M), characterized by an X-ray powder diffractogram as substantially shown in FIG. 16A.

### Compound I Free Acid Form N

In one embodiment, provided is 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one free acid Form N (Compound I free acid Form N), characterized by an X-ray powder diffractogram comprising the following peaks expressed in ±0.2 degrees 2-theta selected from: 4.6, 6.3, and 7.2, as determined on a diffractometer using Cu-Kα radiation.

In some embodiments, Compound I free acid Form N is further characterized by an X-ray powder diffractogram comprising one or more additional peaks expressed in ±0.2 degrees 2-theta selected from: 9.2, 11.9, 16.1, 18.6, 20.4, and 21.0, as determined on a diffractometer using Cu-Kα radiation.

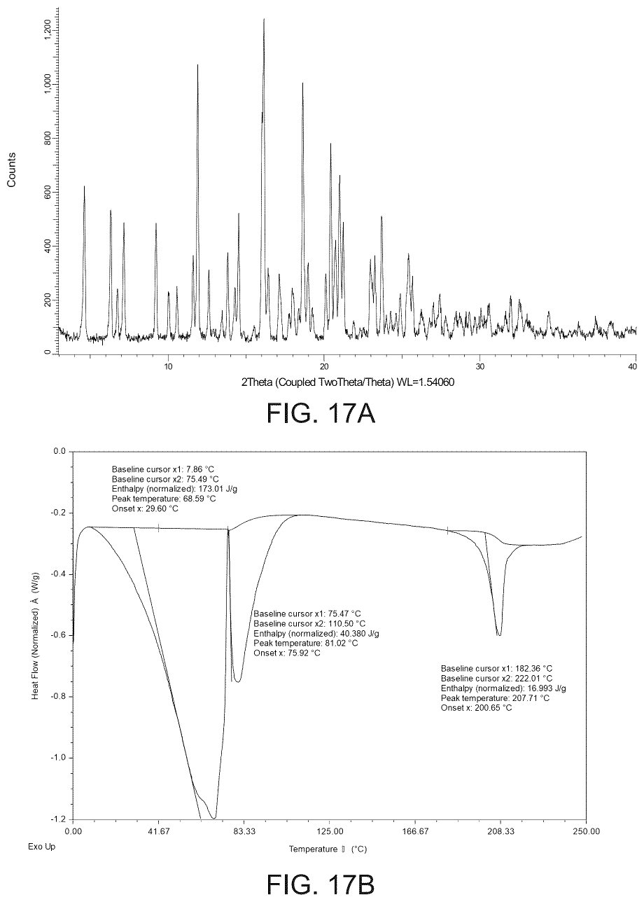

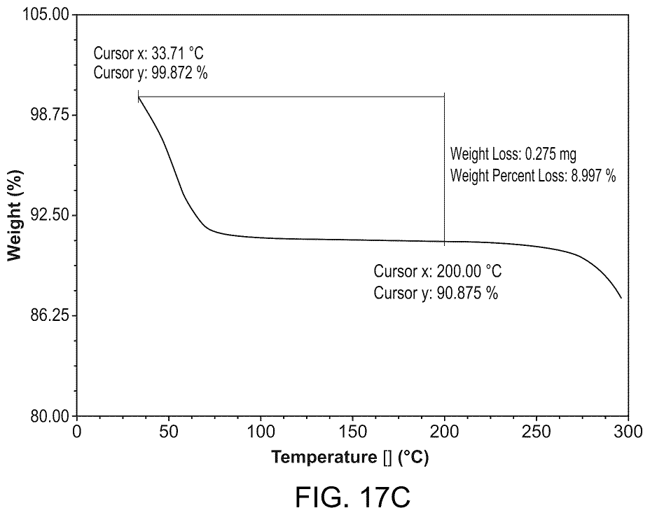

In some embodiments, Compound I free acid Form N is further characterized by an X-ray powder diffractogram as substantially shown in FIG. 17A.

In some embodiments, Compound I free acid Form N is further characterized by a DSC comprising endotherms at about 68.6° C. (peak), about 81.0° C. (peak), and about 207.7° C. (peak). In some embodiments, crystalline Compound I free acid Form N is further characterized by a DSC as substantially shown in FIG. 17B.

In some embodiments, Compound I free acid Form N is obtained equilibration of free acid Form A in ACN/water (9:1 v/v) at room temperature for 2 weeks.

In some embodiments, Compound I free acid Form N is further characterized by TGA showing a weight loss of about 9.0% up to about 200° C.

In some embodiments, Compound I free acid Form N is a hydrate. In some embodiments, the molar ratio of water/Compound I free acid in Compound I free acid Form N is 5.8 (10.3 wt %).

### Compound I Sodium Salt Form A

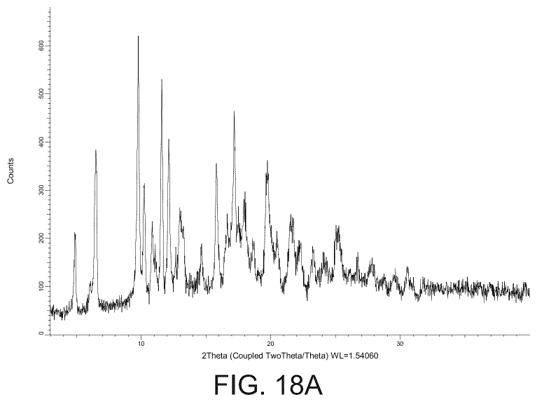

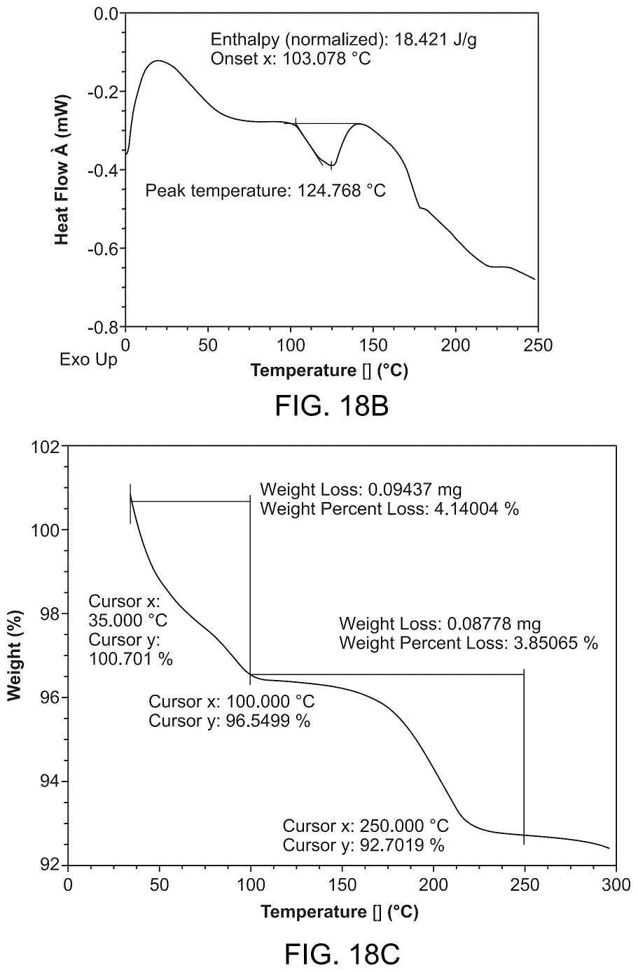

In one embodiment, provided is crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one sodium salt Form A (Compound I sodium salt Form A). In some embodiments, Compound I sodium salt Form A is characterized by an X-ray powder diffractogram as substantially shown in FIG. 18A.

In some embodiments, Compound I sodium salt Form A is further characterized by a DSC comprising an endotherm at about 124.8° C. (peak). In some embodiments, crystalline Compound I sodium salt Form A is further characterized by a DSC as substantially shown in FIG. 18B.

In some embodiments, Compound I sodium salt Form A is obtained via slurry Compound I free acid Form A and equimolar NaOH in acetone at room temperature for 4 days.

In some embodiments, Compound I sodium salt Form A is further characterized by TGA showing a two-step weight loss of about 4.1% up to about 100° C. and about 3.9% from about 100-250° C.

In some embodiments, crystalline Compound I sodium salt Form A is a solvate. In some embodiments, crystalline Compound I sodium salt Form A is an acetone solvate. In some embodiments, crystalline Compound I sodium salt Form A is an acetone-water hetero solvate. In some embodiments, the Compound I sodium salt Form A has a molar ratio of acetone/Compound I free acid of 0.6 (3.8 wt %). In some embodiments, the Compound I sodium salt Form A has a molar ratio of water/Compound I free acid of 2.1 (4.0 wt %). In some embodiments, the molar ratio of sodium/Compound I free acid in crystalline Compound I sodium salt Form A is 1:1.

### Compound I Sodium Salt Form B

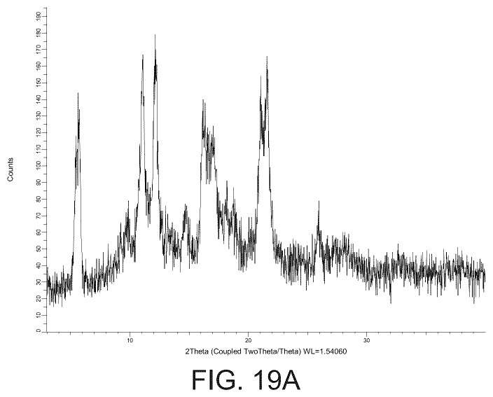

In one embodiment, provided is crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one sodium salt Form B (Compound I sodium salt Form B). In some embodiments, Compound I sodium salt Form B is characterized by an X-ray powder diffractogram as substantially shown in FIG. 19A.

In some embodiments, Compound I sodium salt Form B is obtained via slurry Compound I free acid Form A and equimolar NaOH in THF at room temperature for 4 days, then slowly cooled to 5° C. and stirred for 2 days, followed by addition of antisolvent (water).

### Compound I Potassium Salt Form A

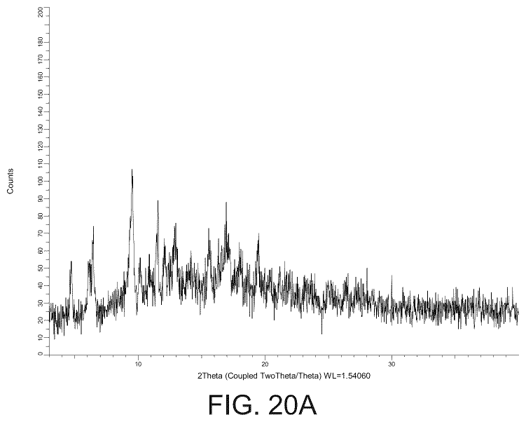

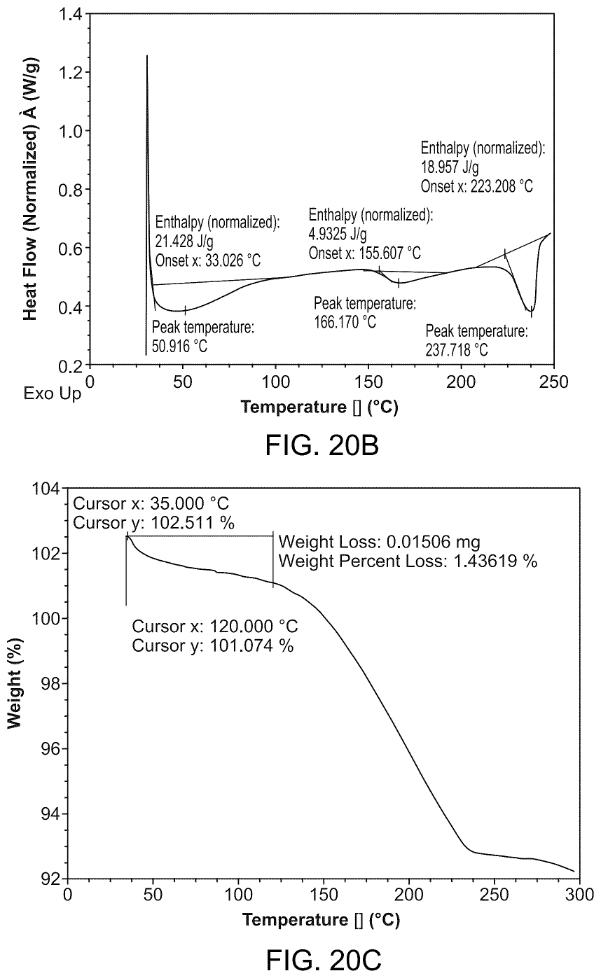

In one embodiment, provided is crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one potassium salt Form A (Compound I potassium salt Form A). In some embodiments, Compound I potassium salt Form A is characterized by an X-ray powder diffractogram as substantially shown in FIG. 20A.

In some embodiments, Compound I potassium salt Form A is further characterized by a DSC comprising endotherms at about 50.9° C. (peak), about 166.2° C. (peak), and about 237.7° C. (peak). In some embodiments, crystalline Compound I potassium salt Form A is further characterized by a DSC as substantially shown in FIG. 20B.

In some embodiments, Compound I potassium salt Form A is obtained via slurry Compound I free acid Form A and equimolar KOH in acetone at room temperature for 4 days, then slowly cooled to 5° C. and stirred for 2 days, followed by addition of antisolvent (MTBE).

In some embodiments, Compound I potassium salt Form A is further characterized by TGA showing a weight loss of about 1.4% up to about 130° C.

In some embodiments, crystalline Compound I potassium salt Form A is a solvate. In some embodiments, crystalline Compound I potassium salt Form A is an acetone solvate. In some embodiments, crystalline Compound I potassium salt Form A is a MTBE solvate. In some embodiments, crystalline Compound I potassium salt Form A is an acetone-MTBE hetero solvate. In some embodiments, the Compound I potassium salt Form A has a molar ratio of acetone/Compound I free acid of 0.04 (0.3 wt %). In some embodiments, the Compound I potassium salt Form A has a molar ratio of MTBE/Compound I free acid of 0.45 (4.1 wt %). In some embodiments, the molar ratio of potassium/Compound I free acid in crystalline Compound I sodium salt Form A is 0.9:1.

### Compositions

In some embodiments, provided is a composition comprising a salt or crystalline Form of 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one (Compound I), or salt or solvate thereof, as described herein.

In one embodiment, provided is a composition comprising a salt or crystalline Form of 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one (Compound I), or salt or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in a composition is in the designated salt, crystalline Form, or crystalline salt Form.

In one embodiment, provided is a composition comprising 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one L-arginine salt (Compound I L-arginine salt), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I L-arginine salt.

In one embodiment, provided is a composition comprising crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one L-arginine salt Form B (Compound I L-arginine salt Form B), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I L-arginine salt Form B.

In one embodiment, provided is a composition comprising crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one L-arginine salt Form A (Compound I L-arginine salt Form A), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I L-arginine salt Form A.

In one embodiment, provided is a composition comprising crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one L-arginine salt Form C (Compound I L-arginine salt Form C), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I L-arginine salt Form C.

In one embodiment, provided is a composition comprising crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one free acid Form A (Compound I free acid Form A), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I free acid Form A.

In one embodiment, provided is a composition comprising crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one free acid Form B (Compound I free acid Form B), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I free acid Form B.

In one embodiment, provided is a composition comprising crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one free acid Form C (Compound I free acid Form C), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I free acid Form C.

In one embodiment, provided is a composition comprising crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one free acid Form D (Compound I free acid Form D), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I free acid Form D.

In one embodiment, provided is a composition comprising crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one free acid Form E (Compound I free acid Form E), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I free acid Form E.

In one embodiment, provided is a composition comprising crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one free acid Form F (Compound I free acid Form F), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I free acid Form F.

In one embodiment, provided is a composition comprising crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one free acid Form G (Compound I free acid Form G), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I free acid Form G.

In one embodiment, provided is a composition comprising crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one free acid Form H (Compound I free acid Form H), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I free acid Form H.

In one embodiment, provided is a composition comprising crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one free acid Form I (Compound I free acid Form I), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I free acid Form I.

In one embodiment, provided is a composition comprising crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one free acid Form J (Compound I free acid Form J), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I free acid Form J.

In one embodiment, provided is a composition comprising crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one free acid Form K (Compound I free acid Form K), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I free acid Form K.

In one embodiment, provided is a composition comprising crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one free acid Form L (Compound I free acid Form L), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I free acid Form L.

In one embodiment, provided is a composition comprising crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one free acid Form N (Compound I free acid Form N), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I free acid Form N.

In one embodiment, provided is a composition comprising crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one sodium salt Form A (Compound I sodium salt Form A), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I sodium salt Form A.

In one embodiment, provided is a composition comprising crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one sodium salt Form B (Compound I sodium salt Form B), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I sodium salt Form B.

In one embodiment, provided is a composition comprising crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one potassium salt Form A (Compound I potassium salt Form A), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I potassium salt Form A.

In some embodiments, the composition is a pharmaceutical composition which further comprises a pharmaceutically acceptable excipient.

In some embodiments, provided is a process for preparing crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one L-arginine salt (Compound I L-arginine salt), comprising contacting Compound I with L-arginine in a solvent for a time sufficient to provide a crystalline Compound I L-arginine salt.

In some embodiments, the solvent is a mixture of IPA/H₂O. In some embodiments, the solvent is a mixture of IPA/H₂O at a ratio of 7:3 v/v.

In some embodiments, the contacting comprises adding 1.1 molar equivalents of L-arginine to Compound I. In some embodiments, the contacting comprises adding 1.1 molar equivalents of L-arginine to Compound I at a temperature of about 10° C. to about 90° C. In some embodiments, the contacting comprises adding 1.1 molar equivalents of L-arginine to Compound I at a temperature of about 30° C. to about 70° C. In some embodiments, the contacting comprises adding 1.1 molar equivalents of L-arginine to Compound I at a temperature of about 50° C. to about 55° C.

In some embodiments, the contacting further comprises adding about 2 wt % of seed crystals to the mixture of Compound I and L-arginine.

In some embodiments, the contacting further comprises adding additional IPA dropwise into the mixture of Compound I and L-arginine. In some embodiments, the contacting further comprises adding about 10 to about 20 molar equivalents of IPA dropwise into the mixture of Compound I and L-arginine.

In some embodiments, the contacting further comprises, following addition of 10 to 20 molar equivalents of IPA, stirring at a temperature of about −10° C. to about 15° C. In some embodiments, the contacting further comprises, following addition of 10 to 20 molar equivalents of IPA, stirring at a temperature of about −0° C. to about 5° C. In some embodiments, the contacting further comprises, following addition of 10 to 20 molar equivalents of IPA, stirring at a temperature of about −0° C. to about 5° C.

In some embodiments, the contacting further comprises adding additional IPA dropwise into the mixture of Compound I and L-arginine and stirring at a temperature of about −10° C. to about 15° C. In some embodiments, the contacting further comprises adding 10 to 20 molar equivalents of additional IPA dropwise into the mixture of Compound I and L-arginine and stirring at a temperature of about −10° C. to about 15° C.

In some embodiments, the process further comprises, following said contacting step, isolating the crystalline Compound I L-arginine salt.

In some embodiments, the isolating comprises the steps of filtering, washing, and drying the crystalline Compound I L-arginine salt.

In some embodiments, provided is a process for preparing crystalline Compound I L-arginine salt wherein at least about 95% of the crystalline Compound I L-arginine salt is Form B.

In some embodiments, provided is a process for preparing crystalline Compound I L-arginine salt wherein at least about 95% of the crystalline Compound I L-arginine salt is Form A.

In some embodiments, provided is a process for preparing crystalline Compound I L-arginine salt wherein at least about 95% of the crystalline Compound I L-arginine salt is Form C.

In some embodiments, provided is a process for preparing crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one sodium salt (Compound I sodium salt), comprising contacting Compound I with sodium hydroxide in a solvent for a time sufficient to provide a crystalline Compound I sodium salt.

In some embodiments, the solvent is acetone.

In some embodiments, the contacting comprises adding an equimolar amount of sodium hydroxide to Compound I. In some embodiments, the contacting comprises adding an equimolar amount of sodium hydroxide to Compound I at a temperature of about 0° C. to about 50° C. In some embodiments, the contacting comprises adding an equimolar amount of sodium hydroxide to Compound I at a temperature of about 20° C. to about 30° C.

In some embodiments, the process further comprises, following said contacting step, isolating the crystalline Compound I sodium salt.

In some embodiments, the isolating comprises the steps of centrifuging and drying the crystalline Compound I sodium salt.

In some embodiments, provided is a process for preparing crystalline Compound I L-arginine salt wherein at least about 95% of the crystalline Compound I sodium salt is Form A.

### Pharmaceutical Compositions and Administration

In some embodiments, a chemical entity (e.g., a salt or crystalline Form of 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one (Compound I), or salt or solvate thereof, as described herein) that modulates (e.g., agonizes) GLP-1 activity is administered as a pharmaceutical composition that includes the chemical entity and one or more pharmaceutically acceptable excipients, and optionally one or more additional therapeutic agents as described herein.

In some embodiments, the chemical entities can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, U K. 2012).

### Routes of Administration and Composition Components

In some embodiments, the chemical entities described herein or a pharmaceutical composition thereof can be administered to subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Compositions can be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In general, the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Pharmacologically acceptable excipients usable in the rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In certain embodiments, suppositories can be prepared by mixing the chemical entities described herein with suitable non-irritating excipients or carriers such as cocoa butter, polyethylene glycol or a suppository wax which are solid at ambient temperature but liquid at body temperature and therefore melt in the rectum and release the active compound. In other embodiments, compositions for rectal administration are in the form of an enema.

In other embodiments, the compounds described herein or a pharmaceutical composition thereof are suitable for local delivery to the digestive or GI tract by way of oral administration (e.g., solid or liquid dosage forms).

Solid dosage forms for oral administration include capsules, tablets, pills, powders, and granules. In such solid dosage forms, the chemical entity is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In one embodiment, the compositions will take the form of a unit dosage form such as a pill or tablet and thus the composition may contain, along with a chemical entity provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more chemical entities provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In certain embodiments the excipients are sterile and generally free of undesirable matter. These compositions can be sterilized by conventional, well-known sterilization techniques. For various oral dosage form excipients such as tablets and capsules, sterility is not required. The USP/NF standard is usually sufficient.

Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

Topical compositions can include ointments and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. As with other carriers or vehicles, an ointment base should be inert, stable, nonirritating and non-sensitizing.

In any of the foregoing embodiments, pharmaceutical compositions described herein can include one or more one or more of the following: lipids, interbilayer crosslinked multilamellar vesicles, biodegradeable poly(D,L-lactic-co-glycolic acid) [PLGA]-based or poly anhydride-based nanoparticles or microparticles, and nanoporous particle-supported lipid bilayers.

### Dosages

The dosages may be varied depending on the requirement of the patient, the severity of the condition being treating and the particular compound being employed. Proper dosage for a particular situation can be determined by one skilled in the medical arts. In some cases, the total daily dosage may be divided and administered in portions throughout the day or by means providing continuous delivery.

In some embodiments, the compounds described herein are administered at a dosage of from about 0.001 mg/Kg to about 500 mg/Kg (e.g., from about 0.001 mg/Kg to about 200 mg/Kg; from about 0.01 mg/Kg to about 200 mg/Kg; from about 0.01 mg/Kg to about 150 mg/Kg; from about 0.01 mg/Kg to about 100 mg/Kg; from about 0.01 mg/Kg to about 50 mg/Kg; from about 0.01 mg/Kg to about 10 mg/Kg; from about 0.01 mg/Kg to about 5 mg/Kg; from about 0.01 mg/Kg to about 1 mg/Kg; from about 0.01 mg/Kg to about 0.5 mg/Kg; from about 0.01 mg/Kg to about 0.1 mg/Kg; from about 0.1 mg/Kg to about 200 mg/Kg; from about 0.1 mg/Kg to about 150 mg/Kg; from about 0.1 mg/Kg to about 100 mg/Kg; from about 0.1 mg/Kg to about 50 mg/Kg; from about 0.1 mg/Kg to about 10 mg/Kg; from about 0.1 mg/Kg to about 5 mg/Kg; from about 0.1 mg/Kg to about 1 mg/Kg; from about 0.1 mg/Kg to about 0.5 mg/Kg).

### Regimens

The foregoing dosages can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In an embodiment, a therapeutic compound is administered to an individual for a period of time followed by a separate period of time. In another embodiment, a therapeutic compound is administered for a first period and a second period following the first period, with administration stopped during the second period, followed by a third period where administration of the therapeutic compound is started and then a fourth period following the third period where administration is stopped. In an aspect of this embodiment, the period of administration of a therapeutic compound followed by a period where administration is stopped is repeated for a determined or undetermined period of time. In a further embodiment, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

### Methods of Treatment

This disclosure features methods for treating a subject (e.g., a human) having a disease, disorder, or condition in which modulation of GLP-1R (e.g., repressed or impaired and/or elevated or unwanted GLP-1R) is beneficial for the treatment of the underlying pathology and/or symptoms and/or progression of the disease, disorder, or condition. In certain embodiments, provided is a method of treating a subject having a disease, disorder, or condition mediated, at least in part, by GLP-1, comprising administering to the subject a compound as described herein, e.g. Compound I L-arginine salt, Form A, B, or C. In certain embodiments, the methods described herein can include or further include treating one or more conditions associated, co-morbid or sequela with any one or more of the conditions described herein.

In some embodiments, the compounds and pharmaceutical compositions and methods for treating a patient described herein induce, by administration of a solid form of Compound I as described herein to the patient in need thereof, one or more of a reduction of blood glucose levels (e.g., reduce blood glucose levels), a reduction of blood hemoglobin A1c (HbA1c) levels, a promotion of insulin synthesis, a stimulation of insulin secretion, an increase in the mass of 3-cells, a modulation of gastric acid secretion, a modulation of gastric emptying, a decrease in the body mass index (BMI), and/or a decrease in glucagon production (e.g., level). In some embodiments, the compounds and pharmaceutical compositions and methods for treating a patient described herein can reduce blood glucose levels, reduce blood hemoglobin A1c (HbA1c) levels, promote insulin synthesis, stimulate insulin secretion, increase the mass of β-cells, modulate gastric acid secretion, modulate gastric emptying, decrease the body mass index (BMI), decrease glucagon production (e.g., level), or any combination thereof. In certain embodiments, the compounds and pharmaceutical compositions and methods for treating a patient described herein stabilize serum glucose and serum insulin levels (e.g., serum glucose and serum insulin concentrations). Also provided herein are methods for modulating glucose or insulin levels in a patient in need of such modulating, the method comprising administering to the patient an effective amount of Compound I, or a pharmaceutically acceptable salt or solvate thereof, or a pharmaceutical composition as disclosed herein.

In some embodiments, provided herein is a method for reducing the risk (e.g., by about at least 20%, at least 30%, at least 40%, at least 50%, at least 60%, at least 70%, or at least 80%) of major adverse cardiovascular events (MACE) in a patient in need thereof, the method comprising administering to the patient an effective amount Compound I, or a pharmaceutically acceptable salt or solvate thereof, or a pharmaceutical composition as disclosed herein. In certain of these embodiments, the patient is an adult that has been diagnosed with type 2 diabetes (T2D). In certain embodiments, the patient is an adult that has been diagnosed with a heart disease. In certain embodiments, the patient is an adult that has been diagnosed with type 2 diabetes (T2D) and a heart disease. In certain embodiments, the patient is an adult that has type 2 diabetes (T2D). In certain embodiments, the patient is an adult that has a heart disease. In certain embodiments, the patient has type 2 diabetes (T2D) and a heart disease.

In some embodiments, the methods described herein further include the step of identifying a patient (e.g., a subject) in need of such treatment (e.g., by way of blood assay, body mass index, or other conventional method known in the art).

In some embodiments, the methods described herein further include the step of identifying a patient (e.g., patient) that has a disease, disorder, or condition as provided here (e.g., a GLP-1 associated disease, disorder, or condition).

In some embodiments, the methods described herein further include the step of identifying a patient (e.g., patient) that has type 2 diabetes mellitus. In some embodiments, determining if the patient has type 2 diabetes mellitus includes performing an assay to determine the level of hemoglobin A1c (HbA1c), fasting plasma glucose, non-fasting plasma glucose, or any combination thereof. In some embodiments, the level of HbA1c is about 6.5% to about 24.0%. In some embodiments, the level of HbA1c is greater than or about 6.5%. In some embodiments, the level of HbA1c is greater than or about 8.0%. In some embodiments, the level of HbA1c is greater than or about 10.0%. In some embodiments, the level of HbA1c is greater than or about 12.0%. In some embodiments, the level of HbA1c is greater than or about 14.0%. In some embodiments, the level of HbA1c is greater than or about 16.0%. In some embodiments, the level of HbA1c is greater than or about 18.0%. In some embodiments, the level of HbA1c is greater than or about 20.0%. In some embodiments, the level of HbA1c is greater than or about 22.0%. In some embodiments, the level of HbA1c is greater than or about 24.0%.

In some embodiments, the level of fasting plasma glucose is greater than or about 120 mg/dL to greater than or about 750 mg/dL. In some embodiments, the level of fasting plasma glucose is greater than or about 200 mg/dL to greater than or about 500 mg/dL. In some embodiments, the level of fasting plasma glucose is greater than or about 300 mg/dL to greater than or about 700 mg/dL.

In some embodiments, the level of non-fasting plasma glucose is greater than or about 190 mg/dL to greater than or about 750 mg/dL. In some embodiments, the level of non-fasting plasma glucose is greater than or about 250 mg/dL to greater than or about 450 mg/dL. In some embodiments, the level of non-fasting plasma glucose is greater than or about 400 mg/dL to greater than or about 700 mg/dL.

In some embodiments, determining if the patient has type 2 diabetes mellitus further includes determining the patient's BMI. In some embodiments, the BMI of the patient is greater than or about 22 kg/m² to greater than or about 100 kg/m². In some embodiments, the BMI of the patient is greater than or about 30 kg/m² to greater than or about 90 kg/m². In some embodiments, the BMI of the patient is greater than or about 40 kg/m² to greater than or about 80 kg/m². In some embodiments, the BMI of the patient is greater than or about 50 kg/m² to greater than or about 70 kg/m².

In some embodiments, additional factors (e.g. risk factors) used for determining if the patient has type 2 diabetes mellitus further includes age and ethnicity of the patient. In some embodiments, the patient's age is greater than or about 10 years. In some embodiments, the patient's age is greater than or about 15 years. In some embodiments, the patient's age is greater than or about 20 years. In some embodiments, the patient's age is greater than or about 25 years. In some embodiments, the patient's age is greater than or about 30 years. In some embodiments, the patient's age is greater than or about 35 years. In some embodiments, the patient's age is greater than or about 40 years. In some embodiments, the patient's age is greater than or about 42 years. In some embodiments, the patient's age is greater than or about 44 years. In some embodiments, the patient's age is greater than or about 46 years. In some embodiments, the patient's age is greater than or about 48 years. In some embodiments, the patient's age is greater than or about 50 years. In some embodiments, the patient's age is greater than or about 52 years. In some embodiments, the patient's age is greater than or about 54 years. In some embodiments, the patient's age is greater than or about 56 years. In some embodiments, the patient's age is greater than or about 58 years. In some embodiments, the patient's age is greater than or about 60 years. In some embodiments, the patient's age is greater than or about 62 years. In some embodiments, the patient's age is greater than or about 64 years. In some embodiments, the patient's age is greater than or about 66 years. In some embodiments, the patient's age is greater than or about 68 years. In some embodiments, the patient's age is greater than or about 70 years. In some embodiments, the patient's age is greater than or about 72 years. In some embodiments, the patient's age is greater than or about 74 years. In some embodiments, the patient's age is greater than or about 76 years. In some embodiments, the patient's age is greater than or about 78 years. In some embodiments, the patient's age is greater than or about 80 years. In some embodiments, the patient's age is greater than or about 85 years. In some embodiments, the patient's age is greater than or about 90 years. In some embodiments, the patient's age is greater than or about 95 years. In some embodiments, the ethnicity of the patient may be African American, American Indian or Alaska Native, Asian American, Hispanics or Latinos, or Native Hawaiian, or Pacific Islander.

In some embodiments, the patient is a pediatric patient. The term “pediatric patient” as used herein refers to a patient under the age of 21 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W.B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994. In some embodiments, a pediatric patient is from birth through the first 28 days of life, from 29 days of age to less than two years of age, from two years of age to less than 12 years of age, or 12 years of age through 21 years of age (up to, but not including, the twenty-second birthday). In some embodiments, a pediatric patient is from birth through the first 28 days of life, from 29 days of age to less than 1 year of age, from one month of age to less than four months of age, from three months of age to less than seven months of age, from six months of age to less than 1 year of age, from 1 year of age to less than 2 years of age, from 2 years of age to less than 3 years of age, from 2 years of age to less than seven years of age, from 3 years of age to less than 5 years of age, from 5 years of age to less than 10 years of age, from 6 years of age to less than 13 years of age, from 10 years of age to less than 15 years of age, or from 15 years of age to less than 22 years of age. In some embodiments, the patient is an adult patient.

### Indications

### Obesity

In some embodiments, the condition, disease or disorder is obesity and conditions, diseases or disorders that are associated with or related to obesity. Non-limiting examples of obesity and obesity related conditions include symptomatic obesity, simple obesity, childhood obesity, morbid obesity, and abdominal obesity (central obesity characterized by abdominal adiposity). Non-limiting examples of symptomatic obesity include endocrine obesity (e.g., Cushing syndrome, hypothyroidism, insulinoma, obese type II diabetes, pseudohypoparathyroidism, hypogonadism), hypothalamic obesity, hereditary obesity (e.g., Prader-Willi syndrome, Laurence-Moon-Biedl syndrome), and drug-induced obesity (e.g., steroid, phenothiazine, insulin, sulfonylurea agent, or β-blocker-induced obesity).

In some embodiments, the condition, disease or disorder is associated with obesity. Examples of such conditions, diseases or disorders include, without limitation, glucose tolerance disorders, diabetes (e.g., type 2 diabetes, obese diabetes), lipid metabolism abnormality, hyperlipidemia, hypertension, cardiac failure, hyperuricemia, gout, fatty liver (including non-alcoholic steatohepatitis (NASH)), coronary heart disease (e.g., myocardial infarction, angina pectoris), cerebral infarction (e.g., brain thrombosis, transient cerebral ischemic attack), bone or articular disease (e.g., knee osteoarthritis, hip osteoarthritis, spondylitis deformans, lumbago), sleep apnea syndrome, obesity hypoventilation syndrome (Pickwickian syndrome), menstrual disorder (e.g., abnormal menstrual cycle, abnormality of menstrual flow and cycle, amenorrhea, abnormal catamenial symptom), visceral obesity syndrome, urine incontinence, and metabolic syndrome. In some embodiments, the chemical compound and pharmaceutical compositions described herein can be used to treat patients exhibiting symptoms of both obesity and insulin deficiency.

### Diabetes

In some embodiments, the condition, disease or disorder is diabetes. Non-limiting examples of diabetes include type 1 diabetes mellitus, type 2 diabetes mellitus (e.g., diet-treated type 2-diabetes, sulfonylurea-treated type 2-diabetes, a far-advanced stage type 2-diabetes, long-term insulin-treated type 2-diabetes), diabetes mellitus (e.g., non-insulin-dependent diabetes mellitus, insulin-dependent diabetes mellitus), gestational diabetes, obese diabetes, autoimmune diabetes, and borderline type diabetes. In some embodiments, the condition, disease or disorder is type 2 diabetes mellitus (e.g., diet-treated type 2-diabetes, sulfonylurea-treated type 2-diabetes, a far-advanced stage type 2-diabetes, long-term insulin-treated type 2-diabetes).

In some embodiments, the condition, disease or disorder is associated with diabetes (e.g., a complication of diabetes). Non-limiting examples of disorders associated with diabetes include obesity, obesity-related disorders, metabolic syndrome, neuropathy, nephropathy (e.g., diabetic nephropathy), retinopathy, diabetic cardiomyopathy, cataract, macroangiopathy, osteopenia, hyperosmolar diabetic coma, infectious disease (e.g., respiratory infection, urinary tract infection, gastrointestinal infection, dermal soft tissue infections, inferior limb infection), diabetic gangrene, xerostomia, hypacusis, cerebrovascular disorder, diabetic cachexia, delayed wound healing, diabetic dyslipidemia peripheral blood circulation disorder, cardiovascular risk factors. (e.g., coronary artery disease, peripheral artery disease, cerebrovascular disease, hypertension, and risk factors related to unmanaged cholesterol and/or lipid levels, and/or inflammation), NASH, bone fracture, and cognitive dysfunction.

Other non-limiting examples of disorders related to diabetes include pre-diabetes, hyperlipidemia (e.g., hypertriglyceridemia, hypercholesterolemia, high LDL-cholesterolemia, low HDL-cholesterolemia, postprandial hyperlipemia), metabolic syndrome (e.g., metabolic disorder where activation of GLP-1R is beneficial, metabolic syndrome X), hypertension, impaired glucose tolerance (IGT), insulin resistance, and sarcopenia.

In some embodiments, the condition, disease or disorder is diabetes and obesity (diabesity). In some embodiments, the compounds described herein are useful in improving the therapeutic effectiveness of metformin.

### Disorders of Metabolically Important Tissues

In some embodiments, the condition, disease or disorder is a disorder of a metabolically important tissue. Non-limiting examples of metabolically important tissues include liver, fat, pancreas, kidney, and gut.

In some embodiments, the condition, disease or disorder is a fatty liver disease. Fatty liver diseases include, but are not limited to, non-alcoholic fatty acid liver disease (NAFLD), steatohepatitis, non-alcoholic steatohepatitis (NASH), fatty liver disease resulting from hepatitis, fatty liver disease resulting from obesity, fatty liver disease resulting from diabetes, fatty liver disease resulting from insulin resistance, fatty liver disease resulting from hypertriglyceridemia, Abetalipoproteinemia, hyperlipoproteinemia, glycogen storage diseases, Weber-Christian disease, Wolman disease, acute fatty liver of pregnancy, and lipodystrophy.

Non-alcoholic fatty liver disease (NAFLD) represents a spectrum of disease occurring in the absence of alcohol abuse and is typically characterized by the presence of steatosis (fat in the liver). NAFLD is believed to be linked to a variety of conditions, e.g., metabolic syndrome (including obesity, diabetes and hypertriglyceridemia) and insulin resistance. It can cause liver disease in adults and children and can ultimately lead to cirrhosis (Skelly et al., J Hepatol 2001; 35: 195-9; Chitturi et al., Hepatology 2002; 35(2):373-9). The severity of NAFLD ranges from the relatively benign isolated predominantly macrovesicular steatosis (i.e., nonalcoholic fatty liver or NAFL) to non-alcoholic steatohepatitis (NASH) (Angulo et al., J Gastroenterol Hepatol 2002; 17 Suppl:S186-90).

Other non-limiting examples of disorders in metabolically important tissues include joint disorders (e.g., osteoarthritis, secondary osteoarthritis), steatosis (e.g., in the liver); fibrosis (e.g., in the liver); cirrhosis (e.g., in the liver); gall stones; gallbladder disorders; gastroesophageal reflux; sleep apnea; hepatitis; fatty liver; bone disorder characterized by altered bone metabolism, such as osteoporosis, including post-menopausal osteoporosis, poor bone strength, osteopenia, Paget's disease, osteolytic metastasis in cancer patients, osteodistrophy in liver disease and the altered bone metabolism caused by renal failure or haemodialysis, bone fracture, bone surgery, aging, pregnancy, protection against bone fractures, and malnutrition polycystic ovary syndrome; renal disease (e.g., chronic renal failure, glomerulonephritis, glomerulosclerosis, nephrotic syndrome, hypertensive nephrosclerosis, end-stage renal disease); muscular dystrophy, angina pectoris, acute or chronic diarrhea, testicular dysfunction, respiratory dysfunction, frailty, sexual dysfunction (e.g., erectile dysfunction), and geriatric syndrome. In some embodiments, the compounds and pharmaceutical compositions described herein can be used for treating surgical trauma by improving recovery after surgery and/or by preventing the catabolic reaction caused by surgical trauma.

### Cardiovascular and Vascular Diseases

In some embodiments, the condition, disease or disorder is a cardiovascular disease. Non-limiting examples of cardiovascular disease include congestive heart failure, atherosclerosis, arteriosclerosis, coronary heart disease, coronary artery disease, congestive heart failure, coronary heart disease, hypertension, cardiac failure, cerebrovascular disorder (e.g., cerebral infarction), vascular dysfunction, myocardial infarction, elevated blood pressure (e.g., 130/85 mm Hg or higher), and prothrombotic state (exemplified by high fibrinogen or plasminogen activator inhibitor in the blood).

In some embodiments, the condition, disease or disorder is related to a vascular disease. Non-limiting examples of vascular diseases include peripheral vascular disease, macrovascular complications (e.g., stroke), vascular dysfunction, peripheral artery disease, abdominal aortic aneurysm, carotid artery disease, cerebrovascular disorder (e.g., cerebral infarction), pulmonary embolism, chronic venous insufficiency, critical limb ischemia, retinopathy, nephropathy, and neuropathy.

### Neurological Diseases

In some embodiments, the condition, disease or disorder is a neurological disorder (e.g., neurodegenerative disorder) or a psychiatric disorder. Non-limiting examples of neurological disorders include idiopathic intracranial hypertension (IIH), brain insulin resistance, mild cognitive impairment (MCI), Alzheimer's disease (AD), Parkinson's disease (PD), anxiety, dementia (e.g., senile dementia), traumatic brain injury, Huntington's chores, tardive dyskinesia, hyperkinesia, mania, Morbus Parkinson, steel-Richard syndrome, Down's syndrome, myasthenia gravis, nerve trauma, brain trauma, vascular amyloidosis, cerebral hemorrhage I with amyloidosis, brain inflammation, Friedrich's ataxia, acute confusion disorder, amyotrophic lateral sclerosis (ALS), glaucoma, and apoptosis-mediated degenerative diseases of the central nervous system (e.g., Creutzfeld-Jakob Disease, bovine spongiform encephalopathy (mad cow disease), and chronic wasting syndrome). See, e.g., U.S. Publication No. 20060275288A1.

In some embodiments, the condition, disease or disorder is idiopathic intracranial hypertension. Idiopathic intracranial hypertension is characterized by increased intracranial pressure and papilloedema. See, e.g., Virdee et al. Ophthalmol Ther. 2020; 9(4):767-781. In some embodiments, the compounds and pharmaceutical compositions and methods described herein reduce cerebrospinal fluid secretion in a patient with idiopathic intracranial hypertension. In some embodiments, the compounds and pharmaceutical compositions and methods described herein reduce intracranial pressure in a patient with idiopathic intracranial hypertension. In some embodiments, the compounds and pharmaceutical compositions and methods described herein reduce one or more symptoms in a patient with idiopathic intracranial hypertension. Symptoms of idiopathic intracranial hypertension can include severe headaches and visual impairment. In some embodiments, the patient with idiopathic intracranial hypertension is female. In some embodiments, the patient with idiopathic intracranial hypertension is about 20 to about 30 years old. In some embodiments, the patient with idiopathic intracranial hypertension is obese.

In some embodiments, the condition, disease or disorder is Wolfram syndrome. Wolfram syndrome is caused by biallelic mutations of the Wolframin ER transmembrane glycoprotein (Wfs1) gene. See, e.g., Seppa et al. Sci Rep 9, 15742 (2019). Wolfram syndrome can first appear as diabetes mellitus, followed by optic nerve atrophy, deafness, and symptoms of neurodegeneration. Patients with Wolfram syndrome can have symptoms of ataxia, sleep apnea, dysphagia, hearing loss, and loss of taste due to brainstem atrophy. In some embodiments, the compounds and pharmaceutical compositions and methods described herein reduce neuroinflammation in a patient with Wolfram syndrome. In some embodiments, the neuroinflammation is reduced in the inferior olive in the patient. In some embodiments, the compounds and pharmaceutical compositions and methods described herein reduce retinal ganglion cell death in a patient with Wolfram syndrome. In some embodiments, the compounds and pharmaceutical compositions and methods described herein reduce axonal degeneration in a patient with Wolfram syndrome. In some embodiments, the compounds and pharmaceutical compositions and methods described herein reduce one or more symptoms (e.g., any of the symptoms described herein) in a patient with Wolfram syndrome.

Non-limiting examples of psychiatric disorders include drug dependence/addiction (narcotics and amphetamines and attention deficit/hyperactivity disorder (ADHD). The compounds and pharmaceutical compositions described herein can be useful in improving behavioral response to addictive drugs, decreasing drug dependence, prevention drug abuse relapse, and relieving anxiety caused by the absence of a given addictive substance. See, e.g., U.S. Publication No. 20120021979A1.

In some embodiments, the compounds and pharmaceutical compositions described herein are useful in improving learning and memory by enhancing neuronal plasticity and facilitation of cellular differentiation, and also in preserving dopamine neurons and motor function in Morbus Parkinson.

### Insulin-Related

In some embodiments, the condition, disease or disorder is impaired fasting glucose (IFG), impaired fasting glycemia (IFG), hyperglycemia, insulin resistance (impaired glucose homeostasis), hyperinsulinemia, elevated blood levels of fatty acids or glycerol, a hypoglycemic condition, insulin resistant syndrome, paresthesia caused by hyperinsulinemia, hyperlipidaemia, hypercholesteremia, impaired wound healing, leptin resistance, glucose intolerance, increased fasting glucose, dyslipidemia (e.g., hyperlipidemia, atherogenic dyslipidemia characterized by high triglycerides and low HDL cholesterol), glucagonoma, hyperuricacidemia, hypoglycemia (e.g., nighttime hypoglycemia), and concomitant comatose endpoint associated with insulin.

In some embodiments, the compounds and pharmaceutical compositions described herein can reduce or slow down the progression of borderline type, impaired fasting glucose or impaired fasting glycemia into diabetes.

### Autoimmune Disorders

In some embodiments, the condition, disease or disorder is an autoimmune disorder. Non-limiting examples of autoimmune disorders include multiple sclerosis, experimental autoimmune encephalomyelitis, autoimmune disorder is associated with immune rejection, graft versus host disease, uveitis, optic neuropathies, optic neuritis, transverse myelitis, inflammatory bowel disease, rheumatoid arthritis, ankylosing spondylitis, systemic lupus erythematosus, myasthenia gravis, and Graves disease. See, e.g., U.S. Publication No. 20120148586A1.

### Stomach and Intestine-Related Disorders

In some embodiments, the condition, disease or disorder is a stomach or intestine related disorder. Non-limiting examples of these disorders include ulcers of any etiology (e.g. peptic ulcers, Zollinger-Ellison syndrome, drug-induced ulcers, ulcers related to infections or other pathogens), digestion disorders, malabsorption, short bowel syndrome, cul-de-sac syndrome, inflammatory bowel diseases (Crohn's disease and ulcerative colitis), celiac sprue, hypogammaglobulinemic sprue, chemotherapy and/or radiation therapy-induced mucositis and diarrhea, gastrointestinal inflammation, short bowel syndrome, colitis ulcerosa, gastric mucosal injury (e.g., gastric mucosal injury caused by aspirin), small intestinal mucosal injury, and cachexia (e.g., cancerous cachexia, tuberculous cachexia, cachexia associated with blood disease, cachexia associated with endocrine disease, cachexia associated with infectious disease, and cachexia caused by acquired immunodeficiency syndrome).

### Body Weight

In some embodiments, the compounds and pharmaceutical compositions described herein can be used to reduce body weight (e.g., excess body weight), prevent body weight gain, induce weight loss, decrease body fat, or reduce food intake in a patient (e.g., a patient in need thereof). In some embodiments, the weight increase in a patient may be attributed to excessive ingestion of food or unbalanced diets, or may be weight increase derived from a concomitant drug (e.g., insulin sensitizers having a PPARγ agonist-like action, such as troglitazone, rosiglitazone, englitazone, ciglitazone, pioglitazone and the like). In some embodiments, the weight increase may be weight increase before reaching obesity, or may be weight increase in an obese patient. In some embodiments, the weight increase may also be medication-induced weight gain or weight gain subsequent to cessation of smoking. In some embodiments, the weight gain is induced by the use of steroids or antipsychotics.

In some embodiments, the condition, disease or disorder is an eating disorder, such as hyperphagia, binge eating, bulimia, compulsive eating, or syndromic obesity such as Prader-Willi and Bardet-Biedl syndromes.

### Inflammatory Diseases

In some embodiments, the condition, disease or disorder is an inflammatory disorder. Non-limiting examples of inflammatory disorders include chronic rheumatoid arthritis, spondylitis deformans, arthritis deformans, lumbago, gout, post-operational or post-traumatic inflammation, bloating, neuralgia, laryngopharyngitis, cystitis, pneumonia, pancreatitis, enteritis, inflammatory bowel disease (including inflammatory large bowel disease), inflammation in metabolically important tissues including liver, fat, pancreas, kidney and gut, and a proinflammatory state (e.g., elevated levels of proinflammatory cytokines or markers of inflammation-like C-reactive protein in the blood).

### Cancer

In some embodiments, the condition, disease or disorder is cancer. Suitable examples of cancer include breast cancer (e.g., invasive ductal breast cancer, noninvasive ductal breast cancer, inflammatory breast cancer), prostate cancer (e.g., hormone-dependent prostate cancer, hormone-independent prostate cancer), pancreatic cancer (e.g., ductal pancreatic cancer), gastric cancer (e.g., papillary adenocarcinoma, mucous adenocarcinoma, adenosquamous carcinoma), lung cancer (e.g., non-small cell lung cancer, small-cell lung cancer, malignant mesothelioma), colon cancer (e.g., gastrointestinal stromal tumor), rectal cancer (e.g., gastrointestinal stromal tumor), colorectal cancer (e.g., familial colorectal cancer, hereditary non-polyposis colorectal cancer, gastrointestinal stromal tumor), small intestinal cancer (e.g., non-Hodgkin's lymphoma, gastrointestinal stromal tumor), esophageal cancer, duodenal cancer, tongue cancer, pharyngeal cancer (e.g., nasopharyngeal cancer, oropharynx cancer, hypopharyngeal cancer), salivary gland cancer, brain tumor (e.g., pineal astrocytoma, pilocytic astrocytoma, diffuse astrocytoma, anaplastic astrocytoma), neurilemmoma, liver cancer (e.g., primary liver cancer, extrahepatic bile duct cancer), renal cancer (e.g., renal cell cancer, transitional cell cancer of the renal pelvis and ureter), bile duct cancer, endometrial cancer, uterine cervical cancer, ovarian cancer (e.g., epithelial ovarian cancer, extragonadal germ cell tumor, ovarian germ cell tumor, ovarian tumor of low malignant potential), bladder cancer, urethral cancer, skin cancer (e.g., intraocular (ocular) melanoma, Merkel cell carcinoma), hemangioma, malignant lymphoma, malignant melanoma, thyroid cancer (e.g., medullary thyroid cancer), parathyroid cancer, nasal cavity cancer, sinus cancer, bone tumor (e.g., osteosarcoma, Ewing tumor, uterine sarcoma, soft tissue sarcoma), angiofibroma, sarcoma of the retina, penis cancer, testicular tumor, pediatric solid tumor (e.g., Wilms' tumor, childhood kidney tumor), Kaposi's sarcoma, Kaposi's sarcoma caused by AIDS, tumor of maxillary sinus, fibrous histiocytoma, leiomyosarcoma, rhabdomyosarcoma, and leukemia (e.g., acute myeloid leukemia, acute lymphoblastic leukemia).

### Hypothalamic-Pituitary Disorders

In some embodiments, the condition, disease or disorder is related to the hypothalamic-pituitary-gonadal axis. For example, the condition, disease or disorder is related to the hypothalamus-pituitary-ovary axis. In another example, the condition, disease or disorder is related to the hypothalamus-pituitary-testis axis. Hypothalamic-pituitary-gonadal axis diseases include, but are not limited to, hypogonadism, polycystic ovary syndrome, hypothyroidism, hypopituitarism, sexual dysfunction, and Cushing's disease.

In some embodiments, the condition, disease or disorder associated with diabetes is related to the hypothalamic-pituitary-gonadal axis.

### Pulmonary Disease

In some embodiments, the condition, disease or disorder is related to a pulmonary disease. Pulmonary diseases include, but are not limited to, asthma, idiopathic pulmonary fibrosis, pulmonary hypertension, obstructive sleep apnoea-hypopnoea syndrome, and chronic obstructive pulmonary disease (COPD) (e.g., emphysema, chronic bronchitis, and refractory (non-reversible) asthma).

In some embodiments, the condition, disease or disorder associated with diabetes is a pulmonary disease.

### Combination Therapy

In some embodiments, this disclosure contemplates both monotherapy regimens as well as combination therapy regimens.

In some embodiments, the methods described herein can further include administering one or more additional therapies (e.g., one or more additional therapeutic agents and/or one or more therapeutic regimens) in combination with administration of the compounds described herein.

In some embodiments, the methods described herein include administering a compound described herein in combination with one or more of a diet therapy (e.g., dietary monitoring, diet therapy for diabetes), an exercise therapy (e.g., physical activity), blood sugar monitoring, gastric electrical stimulation (e.g., TANTALUS®), and diet modifications.

In some embodiments, the compounds, or a pharmaceutically acceptable salt or solvate thereof as described herein can be administered in combination with one or more additional therapeutic agents.

Representative additional therapeutic agents include, but are not limited to, anti-obesity agents, therapeutic agents for diabetes, therapeutic agents for diabetic complications, therapeutic agents for hyperlipidemia, antihypertensive agents, diuretics, chemotherapeutics, immunotherapeutics, anti-inflammatory drugs, antithrombotic agents, anti-oxidants, therapeutic agents for osteoporosis, vitamins, antidementia drugs, erectile dysfunction drugs, therapeutic drugs for urinary frequency or urinary incontinence, therapeutic agents for NAFLD, therapeutic agents for NASH, and therapeutic agents for dysuria.

In some embodiments, the one or more additional therapeutic agents include those useful, for example, as anti-obesity agents. Non-limiting examples include monoamine uptake inhibitors (e.g., tramadol, phentermine, sibutramine, mazindol, fluoxetine, tesofensine), serotonin 2C receptor agonists (e.g., lorcaserin), serotonin 6 receptor antagonists, histamine H3 receptor modulator, GABA modulator (e.g., topiramate), including GABA receptor agonists (e.g., gabapentin, pregabalin), neuropeptide Y antagonists (e.g., velneperit), peptide YY or an analogue thereof, cannabinoid receptor antagonists (e.g., rimonabant, taranabant), ghrelin antagonists, ghrelin receptor antagonists, ghrelin acylation enzyme inhibitors, opioid receptor antagonists (e.g., GSK-1521498, naltrexone), orexin receptor antagonists, melanocortin 4 receptor agonists, 11β-hydroxysteroid dehydrogenase inhibitors (e.g., AZD-4017, BVT-3498, INCB-13739), pancreatic lipase inhibitors (e.g., orlistat, cetilistat), 03 agonists (e.g., N-5984), diacylglycerol acyltransferase 1 (DGAT1) inhibitors, acetylCoA carboxylase (ACC) inhibitors (e.g., compounds described in WO 2020/234726, WO 2020/044266, and U.S. Pat. No. 8,859,577), stearoyl-CoA desaturated enzyme inhibitors, microsomal triglyceride transfer protein inhibitors (e.g., R-256918), sodium-glucose cotransporter 2 (SGLT-2) inhibitors (e.g., JNJ-28431754, dapagliflozin, AVE2268, TS-033, YM543, TA-7284, ASP1941, remogliflozin, empagliflozin, canagliflozin, ipragliflozin, tofogliflozin, sergliflozin etabonate, remogliflozin etabonate, or ertugliflozin), SGLT-1 inhibitors, MCR-4 agonists, monoamine reuptake inhibitors, melanocytestimulating hormone analogs, 5HT2c agonists, galanin antagonists, anorectic agents (such as a bombesin agonist), thyromimetic agents, dehydroepiandrosterone or analogs thereof, human agouti-related protein (AGRP) inhibitors, neuromedin U agonists, NFK inhibitors (e.g., HE-3286), PPAR agonists (e.g., GFT-505, DRF-11605, gemfibrozil, fenofibrate, balaglitazone, ciglitazone, darglitazone, englitazone, isaglitazone, pioglitazone, rosiglitazone, CLX-0940, GW-1536, GW-1 929, GW-2433, KRP-297, L-796449, LR-90, MK-0767, and SB-21 9994), phosphotyrosine phosphatase inhibitors (e.g., sodium vanadate, trodusquemin), GPR119 agonists (e.g., PSN-821, MBX-2982, APD597, compounds described in WO 2010/140092, WO 2010/128425, WO 2010/128414, WO 2010/106457), glucokinase activators (e.g., piragliatin, AZD-1656, AZD6370, TTP-355, TTP-399, TTP547, ARRY403, MK-0599, TAK-329, AZD5658 or GKM-001 compounds described in WO 2010/103437, WO 2010/103438, WO 2010/013161, WO 2007/122482, WO 2006/112549, WO 2007/028135, WO 2008/047821, WO 2008/050821, WO 2008/136428 and WO 2008/156757), leptin, leptin derivatives (e.g., metreleptin), leptin resistance improving drugs, CNTF (ciliary neurotrophic factor), BDNF (brain-derived neurotrophic factor), cholecystokinin agonists, amylin preparations (e.g., pramlintide, AC-2307), neuropeptide Y agonists (e.g., PYY3-36, derivatives of PYY3-36, obineptide, TM-30339, TM-30335), oxyntomodulin (OXM) preparations, appetite suppressants (e.g. ephedrine), FGF21 preparations (e.g., animal FGF21 preparations extracted from the pancreas of bovine or swine; human FGF21 preparations genetically synthesized using Escherichia coli or yeast; fragments or derivatives of FGF21), anorexigenic agents (e.g., P-57), human proislet peptide (HIP), melanocortin receptor 4 agonist (e.g., setmelanotide), melanin concentrating hormone receptor 1 antagonist, serotonergic agents (e.g. sibutramine, lorcaserin), farnesoid X receptor (FXR) agonist (e.g., obeticholic acid, tropifexor, cilofexor, LY2562175, Met409, TERN-101, EDP305, compounds described in WO 2020/234726 and WO 2020/044266), phentermine, zonisamide, norepinephrine/dopamine reuptake inhibitor (e.g., buproprion), GDF-15 analog, methionine aminopeptidase 2 (MetAP2) inhibitor (e.g., beloranib or ZGN-1061), diethylpropion, phendimetrazine, benzphetamine, fibroblast growth factor receptor (FGFR) modulator, biotin, a MAS receptor modulator, glucagon receptor agonist, CCKa agonists (e.g., compounds described in WO 2005/116034 and U.S. Publication No. 2005/0287100), and AMP-activated protein kinase (AMPK) activator.

In some embodiments, the one or more additional therapeutic agents include those useful, for example, as anti-diabetic agents. Non-limiting examples include insulin and insulin preparations (e.g., animal insulin preparations extracted from the pancreas of bovine or swine; human insulin preparations genetically synthesized using Escherichia coli or yeast; zinc insulin; protamine zinc insulin; fragment or derivative of insulin (e.g., INS-1), oral insulin preparation, synthetic human insulin), insulin sensitizers (e.g., pioglitazone or a salt thereof), biguanides (e.g., metformin, buformin or a salt thereof (e.g., hydrochloride, fumarate, succinate)), glucagon analogs (e.g., any of glucagon analogs described, e.g., in WO 2010/011439), agents which antagonize the actions of or reduce secretion of glucagon, sulfonylurea agents (e.g., chlorpropamide, tolazamide, glimepiride, tolbutamide, glibenclamide, gliclazide, acetohexamide, glyclopyramide, glybuzole, glyburide, glipizide), thiazolidinedione agents (e.g. rosiglitazone, lobeglitazone, troglitazone, balaglitazone, rivoglitazone, lobeglitazone or pioglitazone), glitazars (e.g., aleglitazar, chiglitazar, saroglitazar, muraglitazar, tesaglitazar), SGLT2 inhibitors (e.g., JNJ-28431754, dapagliflozin, AVE2268, TS-033, YM543, TA-7284, ASP1941, THR1474, TS-071, ISIS388626, LX4211, remogliflozin, empagliflozin, canagliflozin, ipragliflozin, tofogliflozin, sergliflozin etabonate, remogliflozin etabonate, ertugliflozin, compounds described in WO 2010/023594), GPR40 agonists (e.g., a FFAR1/FFA1 agonist, e.g. fasiglifam), α-glucosidase inhibitors (e.g., adiposin, camiglibose, pradimicin-Q, salbostatin, voglibose, acarbose, miglitol, emiglitate), insulin secretagogues, such as prandial glucose regulators (sometimes called “short-acting secretagogues”), e.g., meglitinides (e.g. repaglinide and nateglinide), cholinesterase inhibitors (e.g., donepezil, galantamine, rivastigmine, tacrine), NMDA receptor antagonists, dual GLP-1/GIP receptor agonists (e.g., LBT-2000, ZPD1-70), GLP-1R agonists (e.g., exenatide, liraglutide, albiglutide, dulaglutide, abiglutide, taspoglutide, lixisenatide, semaglutide, AVE-0010, S4P and Boc5), and dipeptidyl peptidase IV (DPP-4) inhibitors (e.g., vildagliptin, dutogliptin, gemigliptin, alogliptin, saxagliptin, sitagliptin, linagliptin, berberine, adogliptin, anagliptin (SK-0403), teneligliptin, omarigliptin, B11356, GRC8200, MP-513, PF-00734200, PHX1149, ALS2-0426, TA-6666, TS-021, KRP-104, trelagliptin).

In some embodiments, the one or more additional therapeutic agents include those useful, for example, for treating NAFL and NASH. Non-limiting examples include FXR agonists (e.g., obeticholic acid), PF-05221304, PPAR α/δ agonists (e.g., elafibranor), a synthetic fatty acid-bile conjugate (e.g., aramchol), an anti-lysyl oxidase homologue 2 (LOXL2) monoclonal antibody (e.g., simtuzumab), a caspase inhibitor (e.g., emricasan), a MAPK5 inhibitor (e.g., GS-4997), a galectin 3 inhibitor (e.g., GR-MD-02), a fibroblast growth factor 21 (FGF21) (e.g., BMS-986036), a niacin analogue (e.g., ARJ 3037MO), a leukotriene D4 (LTD4) receptor antagonist (e.g., tipelukast), an acetyl-CoA carboxylase (ACC) inhibitor (e.g., NDI 010976 and compounds described in WO 2009/144554, WO 2003/072197, WO 2009/144555, and WO 2008/065508), a ketohexokinase (KHK) inhibitor (e.g., compounds described in WO 2020/234726), an apoptosis signal-regulating kinase 1 (ASK1) inhibitor, an ileal bile acid transporter (IBAT) inhibitor, a dual antagonist of chemokine receptor 2 (CCR2) and CCR5 (e.g., cenicriviroc), diacylglyceryl acyltransferase 2 (DGAT2) inhibitor (e.g., compounds described in WO 2020/234726 and U.S. Publication No. 20180051012), a CB1 receptor antagonist, an anti-CB1R antibody, glycyrrhizin, schisandra extract, ascorbic acid, glutathione, silymarin, lipoic acid, and d-alpha-tocopherol, ascorbic acid, glutathione, vitamin B-complex, glitazones/thiazolidinediones (e.g., troglitazone, rosiglitazone, pioglitazone, balaglitazone, rivoglitazone, lobeglitazone), metformin, cysteamine, sulfonylureas, alpha-glucosidase inhibitors, meglitinides, vitamin E, tetrahydrolipstatin, milk thistle protein, anti-virals, and anti-oxidants.

In some embodiments, the one or more additional therapeutic agents include those useful, for example, for treating diabetic complications. Non-limiting examples include aldose reductase inhibitors (e.g., tolrestat, epalrestat, zopolrestat, fidarestat, CT-112, ranirestat, lidorestat), neurotrophic factor and increasing agents thereof (e.g., NGF, NT-3, BDNF, neurotrophic production/secretion promoting agents described in WO01/14372 (e.g., 4-(4-chlorophenyl)-2-(2-methyl-1-imidazolyl)-5-[3-(2-methylphenoxyl)propyl]oxazole), compounds described in WO 2004/039365), PKC inhibitors (e.g., ruboxistaurin mesylate), AGE inhibitors (e.g., ALT946, N-phenacylthiazolium bromide (ALT766), EXO-226, pyridorin, pyridoxamine), serotonin and noradrenalin reuptake inhibitors (e.g., duloxetine), sodium channel inhibitors (e.g., lacosamide), active oxygen scavengers (e.g., thioctic acid), cerebral vasodilators (e.g., tiapuride, mexiletine), somatostatin receptor agonists (e.g., BIM23190), and apoptosis signal regulating kinase-1 (ASK-1) inhibitors.

In some embodiments, the one or more additional therapeutic agents include those useful, for example, for treating hyperlipidemia. Non-limiting examples include HMG-COA reductase inhibitors (e.g., pravastatin, simvastatin, lovastatin, atorvastatin, fluvastatin, rosuvastatin, pitavastatin or a salt thereof (e.g., sodium salt, calcium salt)), squalene synthase inhibitors (e.g., compounds described in WO97/10224, e.g., N-[[(3R,5S)-1-(3-acetoxy-2,2-dimethylpropyl)-7-chloro-5-(2,3-dimethoxyphenyl)-2-oxo-1,2,3,5-tetrahydro-4, 1-benzoxazepin-3-yl]acetyl]piperidin-4-acetic acid), fibrate compounds (e.g., bezafibrate, clofibrate, simfibrate, clinofibrate), anion exchange resin (e.g., colestyramine), nicotinic acid drugs (e.g., nicomol, niceritrol, niaspan), phytosterols (e.g., soysterol, gamma oryzanol (γ-oryzanol)), cholesterol absorption inhibitors (e.g., zechia), CETP inhibitors (e.g., dalcetrapib, anacetrapib) and ω-3 fatty acid preparations (e.g., ω-3-fatty acid ethyl esters 90).

In some embodiments, the one or more additional therapeutic agents include those useful, for example, as anti-hypertensive agents. Non-limiting examples include angiotensin converting enzyme inhibitors (e.g., captopril, zofenopril, fbsinopril, enalapril, ceranopril, cilazopril, delapril, pentopril, quinapril, ramipril, lisinopril), angiotensin II antagonists (e.g., candesartan cilexetil, candesartan, losartan, losartan potassium, eprosartan, valsartan, telmisartan, irbesartan, tasosartan, olmesartan, olmesartan medoxomil, azilsartan, azilsartan medoxomil), calcium antagonists (e.g., manidipine, nifedipine, amlodipine, efonidipine, nicardipine, cilnidipine) and_-blockers (e.g., metoprolol, atenolol, propranolol, carvedilol, pindolol). Further non-limiting examples of antihypertensive agents include: diruetics (e.g., chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, torsemide, furosemide, musolimine, bumetanide, triamtrenene, amiloride, spironolactone), alpha adrenergic blockers, beta adrenergic blockers, calcium channel blockers (e.g., diltiazem, verapamil, nifedipine and amlodipine), vasodilators (e.g., hydralazine), renin inhibitors, AT-1 receptor antagonists (e.g., losartan, irbesartan, valsartan), ET receptor antagonists (e.g., sitaxsentan, atrsentan, compounds disclosed in U.S. Pat. Nos. 5,612,359 and 6,043,265), dual ET/AII antagonist (e.g., compounds disclosed in WO 2000/01389), neutral endopeptidase (NEP) inhibitors, If channel blocker ivabradinand, vasopepsidase inhibitors (dual NEP-ACE inhibitors) (e.g., gemopatrilat and nitrates).

In some embodiments, the one or more additional therapeutic agents include those useful, for example, as diuretics. Non-limiting examples include xanthine derivatives (e.g., theobromine sodium salicylate, theobromine calcium salicylate), thiazide preparations (e.g., ethiazide, cyclopenthiazide, trichloromethiazide, hydrochlorothiazide, hydroflumethiazide, benzylhydrochlorothiazide, penfluthiazide, polythiazide, methyclothiazide), antialdosterone preparations (e.g., spironolactone, triamterene), carbonic anhydrase inhibitors (e.g., acetazolamide) and chlorobenzenesulfonamide agents (e.g., chlortalidone, mefruside, indapamide).

In some embodiments, the one or more additional therapeutic agents include those useful, for example, as immunotherapeutic agents. Non-limiting examples include microbial or bacterial compounds (e.g., muramyl dipeptide derivative, picibanil), polysaccharides having immunoenhancing activity (e.g., lentinan, sizofiran, krestin), cytokines obtained by genetic engineering approaches (e.g., interferon, interleukin (IL) such as IL-1, IL-2, IL-12), and colony-stimulating factors (e.g., granulocyte colony-stimulating factor, erythropoietin).

In some embodiments, the one or more additional therapeutic agents include those useful, for example, as anti-thrombotic agents. Non-limiting examples include heparins (e.g., heparin sodium, heparin calcium, enoxaparin sodium, dalteparin sodium) warfarin (e.g., warfarin potassium); anti-thrombin drugs (e.g., aragatroban, dabigatran, boroarginine derivatives, boropeptides, heparins, hirudin, and melagatran), FXa inhibitors (e.g., rivaroxaban, apixaban, edoxaban, YM150, compounds described in WO02/06234, WO 2004/048363, WO 2005/030740, WO 2005/058823, and WO 2005/113504) thrombolytic agents (e.g., anistreplase, streptokinase, tenecteplase (TNK), lanoteplase (nPA), urokinase, tisokinase, alteplase, nateplase, monteplase, pamiteplase, factor VIla inhibitors, PAI-1 inhibitors, alpha2-antiplasmin inhibitors, and anisoylated plasminogen streptokinase activator complex), and platelet aggregation inhibitors (e.g., ticlopidine hydrochloride, clopidogrel, prasugrel, E5555, SHC530348, cilostazol, ethyl icosapentate, beraprost sodium, and sarpogrelate hydrochloride).

In some embodiments, the one or more additional therapeutic agents include those useful, for example, for treating osteoporosis. Non-limiting examples include alfacalcidol, calcitriol, elcatonin, calcitonin salmon, estriol, ipriflavone, pamidronate disodium, alendronate sodium hydrate, incadronate disodium, and risedronate disodium. Suitable examples of vitamins include vitamin B1 and vitamin B12. Suitable examples of erectile dysfunction drugs include apomorphine and sildenafil citrate. Suitable examples of therapeutic agents for urinary frequency or urinary incontinence include flavorxate hydrochloride, oxybutynin hydrochloride and propiverine hydrochloride. Suitable examples of therapeutic agents for dysuria include acetylcholine esterase inhibitors (e.g., distigmine). Suitable examples of anti-inflammatory agents include nonsteroidal anti-inflammatory drugs such as aspirin, acetaminophen, indomethacin.

Other exemplary additional therapeutic agents include agents that modulate hepatic glucose balance (e.g., fructose 1,6-bisphosphatase inhibitors, glycogen phosphorylase inhibitors, glycogen synthase kinase inhibitors, glucokinase activators), agents designed to treat the complications of prolonged hyperglycemia, such as aldose reductase inhibitors (e.g. epalrestat and ranirestat), agents used to treat complications related to micro-angiopathies, anti-dyslipidemia agents, such as HMG-CoA reductase inhibitors (statins, e.g. rosuvastatin pravastatin, pitavastatin, lovastatin, atorvastatin, simvastatin, fluvastatin, itavastatin, ZD-4522), HMG-CoA synthase inhibitors, cholesterol-lowering agents, bile acid sequestrants (e.g., cholestyramine, questran, colestipol, and colesevelam), cholesterol absorption inhibitors (e.g. plant sterols such as phytosterols), cholesteryl ester transfer protein (CETP) inhibitors, inhibitors of the ileal bile acid transport system (IBAT inhibitors), diacylglyceryl acyltransferase 1 (DGAT1) inhibitors (e.g., AZD7687, LCQ908, compounds described in WO 2009/016462, WO 2010/086820), monoacylglycerol O-acyltransferase inhibitors, α-amylase inhibitors (e.g., tendamistat, trestatin, AL-3688), α-glucoside hydrolase inhibitors, SIRT-1 activators, c-Jun N-terminal kinase (JNK) inhibitors, a VPAC2 receptor agonist, TGR5 receptor modulators (e.g., compounds described in), GPBAR1 receptor modulators, GPR120 modulators, high affinity nicotinic acid receptor (HM74A) activators, carnitine palmitoyl transferase enzyme inhibitors, mineralocorticoid receptor inhibitors, inhibitors of TORC2, fatty acid synthetase inhibitors, serine palmitoyl transferase inhibitors, GPR81 modulators, GPR39 modulators, GPR43 modulators, GPR41 modulators, GPR105 modulators, Kv1.3 modulators, retinol binding protein 4 modulators, somatostain receptor modulators, PDHK2 modulators, PDHK4 modulators, MAP4K4 inhibitors, IL1 family modulators (e.g., ILI beta modulators), ACAT inhibitors, MTP inhibitors (e.g., diriotapide, mitratapide, and implitapide), lipooxygenase inhibitors, PCSK9 modulators (e.g., alirocumab and evolocumab), RXRalpha modulators, cysteamine, cystamine, an RNA antisense construct to inhibit protein tyrosine phosphatase PTPRU, vitamin B complex, pentraxin proteins, a protein tyrosine phosphatase-1 B (PTP-1 B) inhibitor (e.g., trodusquemine, hyrtiosal extract, and compounds described by Zhang et al. Drug Discovery Today. 2007, 12(9-10): 373-381), ezitimbe, betaine, pentoxifylline, alpha delta-9 desaturase, BCKDK inhibitors, branched-chain alpha keto acid dehydrogenase kinase (BCBK) inhibitors, PNPLA3 inhibitors, FGF1 9 analogs, SCD1 inhibitors, bile acid binding resins, nicotinic acid (niacin) and analogues thereof, anti-oxidants (e.g., probucol), omega-3 fatty acids, antihypertensive agents, including adrenergic receptor antagonists, such as beta blockers (e.g. atenolol), alpha blockers (e.g. doxazosin), and mixed alpha/beta blockers (e.g. labetalol), adrenergic receptor agonists, including alpha-2 agonists (e.g. clonidine), angiotensin converting enzyme (ACE) inhibitors (e.g. lisinopril), calcium channel blockers, such as dihydropridines (e.g. nifedipine), phenylalkylamines (e.g. verapamil), and benzothiazepines (e.g. diltiazem), angiotensin II receptor antagonists (e.g. candesartan), aldosterone receptor antagonists (e.g. eplerenone, spironolactone), centrally acting adrenergic drugs, such as central alpha agonists (e.g. clonidine), diuretic agents (e.g. furosemide, torsemide, bemetanide, ethacrynic acid, thiazide-type diuretics (e.g., chlorothiazide, hydrochlorothiazide, benzthiazide, hydroflumethiazide, bendroflumethiazide, methychlorthiazide, polythiazide, trichlormethiazide, indapamide), phthalimidine-type diuretics (e.g., chlorthalidone, metolazone), quinazoline-type diuretics (e.g., quinethazone), potassium-sparing diuretics (e.g., triamterene and amiloride), thyroid receptor agonists (e.g., compounds described in WO 2020/117987), haemostasis modulators, including antithrombotics (e.g., activators of fibrinolysis), thrombin antagonists, factor VIIa inhibitors, anticoagulants (e.g., vitamin K antagonists such as warfarin), heparin and low molecular weight analogues thereof, factor Xa inhibitors, and direct thrombin inhibitors (e.g. argatroban), antiplatelet agents (e.g., cyclooxygenase inhibitors (e.g. aspirin), non-steroidal anti-inflammatory drugs (NSAIDS), thromboxane-A2-receptor antagonists (e.g., ifetroban), thromboxane-A2-synthetase inhibitors, PDE inhibitors (e.g., Pletal, dipyridamole)), antagonists of purinergic receptors (e.g., P2Y1 and P2Y12), adenosine diphosphate (ADP) receptor inhibitors (e.g. clopidogrel), phosphodiesterase inhibitors (e.g. cilostazol), glycoprotein IIB/IIA inhibitors (e.g. tirofiban, eptifibatide, and abcixima), adenosine reuptake inhibitors (e.g. dipyridamole), noradrenergic agents (e.g. phentermine), serotonergic agents (e.g. sibutramine, lorcaserin), diacyl glycerolacyltransferase (DGAT) inhibitors, feeding behavior modifying agents, pyruvate dehydrogenase kinase (PDK) modulators, serotonin receptor modulators, monoamine transmission-modulating agents, such as selective serotonin reuptake inhibitors (SSRI) (e.g. fluoxetine), noradrenaline reuptake inhibitors (NARI), noradrenaline-serotonin reuptake inhibitors (SNRI), and monoamine oxidase inhibitors (MAOI) (e.g. toloxatone and amiflamine), compounds described in WO 2007/013694, WO 2007/018314, WO 2008/093639 and WO 2008/099794, GPR40 agonists (e.g., fasiglifam or a hydrate thereof, compounds described in WO 2004/041266, WO 2004/106276, WO 2005/063729, WO 2005/063725, WO 2005/087710, WO 2005/095338, WO 2007/013689 and WO 2008/001931), SGLT1 inhibitors, adiponectin or agonist thereof, IKK inhibitors (e.g., AS-2868), somatostatin receptor agonists, ACC2 inhibitors, cachexia-ameliorating agents, such as a cyclooxygenase inhibitors (e.g., indomethacin), progesterone derivatives (e.g., megestrol acetate), glucocorticoids (e.g., dexamethasone), metoclopramide agents, tetrahydrocannabinol agents, agents for improving fat metabolism (e.g., eicosapentaenoic acid), growth hormones, IGF-1, antibodies against a cachexia-inducing factor TNF-α, LIF, IL-6, and oncostatin M, metabolism-modifying proteins or peptides such as glucokinase (GK), glucokinase regulatory protein (GKRP), uncoupling proteins 2 and 3 (UCP2 and UCP3), peroxisome proliferator-activated receptor a (PPARα), MC4r agonists, insulin receptor agonist, PDE 5 inhibitors, glycation inhibitors (e.g., ALT-711), nerve regeneration-promoting drugs (e.g., Y-128, VX853, prosaptide), antidepressants (e.g., desipramine, amitriptyline, imipramine), antiepileptic drugs (e.g., lamotrigine, trileptal, keppra, zonegran, pregabalin, harkoseride, carbamazepine), antiarrhythmic drugs (e.g., K⁺ channel openers, mexiletine, propafenone, metoprolol, atenolol, carvadiol, propranolol, sotalol, dofetilide, amiodarone, azimilide, ibutilide, ditiazem, and verapamil), acetylcholine receptor ligands (e.g., ABT-594), endothelin receptor antagonists (e.g., ABT-627), narcotic analgesics (e.g., morphine), α2 receptor agonists (e.g., clonidine), local analgesics (e.g., capsaicin), antianxiety drugs (e.g., benzothiazepine), phosphodiesterase inhibitors (e.g., sildenafil), dopamine receptor agonists (e.g., apomorphine), cytotoxic antibodies (e.g., T-cell receptor and IL-2 receptor-specific antibodies), B cell depleting therapies (e.g., anti-CD20 antibody (e.g., rituxan), i-BLyS antibody), drugs affecting T cell migration (e.g., anti-integrin alpha 4/beta 1 antibody (e.g., tysabri), drugs that act on immunophilins (e.g., cyclosporine, tacrolimus, sirolimus, rapamicin), interferons (e.g., IFN-β), immunomodulators (e.g., glatiramer), TNF-binding proteins (e.g., circulating receptors), immunosupressants (e.g., mycophenolate), metaglidasen, AMG-131, balaglitazone, MBX-2044, rivoglitazone, aleglitazar, chiglitazar, saroglitazar, muraglitazar, tesaglitazar, lobeglitazone, PLX-204, PN-2034, GFT-505, THR-0921, exenatide, exendin-4, memantine, midazolam, ketoconazole, ethyl icosapentate, clonidine, azosemide, isosorbide, ethacrynic acid, piretanide, bumetanide, etoposide, piroxicam, NO donating agents (e.g., organonitrates), and NO promoting agents (e.g., phosphodiesterase inhibitors).

In some embodiments, the one or more additional therapeutic agents include those useful, for example, as anti-emetic agents. As used herein, an “anti-emetic” agent refers to any agent that counteracts (e.g., reduces or removes) nausea or emesis (vomiting). It is to be understood that when referring to a therapeutically effective amount of an anti-emetic agent, the amount administered is an amount needed to counteract (e.g., reduce or remove) nausea or emesis (vomiting). While not wishing to be bound by theory, it is believed that administering one or more anti-emetic agents in combination with the formula (I) compounds described herein may allow higher dosages of the formula (I) compounds to be administered, e.g., because the patient may be able to have a normal food intake and thereby respond faster to the treatment.

Non-limiting examples of anti-emetic agents include 5HT3-receptor antagonists (serotonin receptor antagonists), neuroleptics/anti-psychotics, antihistamines, anticholinergic agents, steroids (e.g., corticosteroids), NK1-receptor antagonists (e.g., Neurokinin 1 substance P receptor antagonists), antidopaminergic agents/dopamine receptor antagonists, benzodiazepines, cannabinoids.

For example, the antiemetic agent can be selected from the group consisting of; neuroleptics, antihistamines, anti-cholinergic agents, steroids, 5HT-3-receptor antagonists, NK1-receptor antagonists, anti-dopaminergic agents/dopamine receptor antagonists, benzodiazepines and non-psychoactive cannabinoids.

In some embodiments, the anti-emetic agent is a 5HT3-receptor antagonist (serotonin receptor antagonist). Non-limiting examples of 5HT3-receptor antagonists (serotonin receptor antagonists) include: granisetron (Kytril), dolasetron, ondansetron (Zofran), tropisetron, ramosetron, palonosetron, alosetron, azasetron, bemesetron, zatisetron, batanopirde, MDL-73147EF, metoclopramide, N-3389 (endo-3,9-dimethyl-3,9-diazabicyclo[3,3,1]non-7-yl-1H-indazole-3-carboxamide dihydrochloride), Y-25130 hydrochloride, MDL 72222, Tropanyl-3,5-dimethylbenzoate, 3-(4-allylpiperazin-1-yl)-2-quinoxalinecarbonitrile maleate, zacopride hydrochloride, and mirtazepine. Other non-limiting examples of 5HT3-receptor antagonists (serotonin receptor antagonists) include: cilansetron, clozapine, cyproheptadine, dazopride, hydroxyzine, lerisetron, metoclopramide, mianserin, olanzapine, palonosetron (+netupitant), quetiapine, qamosetron, ramosteron, ricasetron, risperidone, ziprasidone, and zatosetron.

In certain embodiments, the 5HT-3-receptor antagonist is granisetron, dolasetron, ondansetron hydrochloride, tropisetron, ramosetron, palonosetron, alosetron, bemesetron, zatisetron, batanopirde, MDL-73147EF, Metoclopramide, N-3389, Y-25130 hydrochloride, MDL 72222, tropanyl-3,5-dimethylbenzoate, 3-(4-allyl-piperazin-1-yl)-2-quinoxalinecarbonitrile maleate, zacopride hydrochloride and mirtazepine.

In certain embodiments, the 5HT-3-receptor antagonist is granisetron, dolasetron, ondansetron hydrochloride, tropisetron, ramosetron, palonosetron, alosetron, bemesetron, and zatisetron.

In certain embodiments, the 5HT-3-receptor antagonist is granisetron, dolasetron, and ondansetron.

In certain embodiments, the 5HT-3-receptor antagonist is granisetron.

In certain embodiments, the 5HT-3-receptor antagonist is ondansetron.

In some embodiments, the anti-emetic agent is an antihistamine. Non-limiting examples of antihistamines include piperazine derivatives (e.g., cyclizine, meclizine, and cinnarizine); promethazine; dimenhydrinate (Dramamine, Gravol); diphenhydramine; hydroxyzine; buclizine; and Meclizine hydrochloride (Bonine, Antivert), doxylamine, and mirtazapine.

In some embodiments, the anti-emetic agent is an anticholinergic agent (inhibitors of the acetylcholine receptors). Non-limiting examples of anticholinergic agents include: atropine, scopolamine, glycopyrron, hyoscine, artane (Trihexy-5 trihexyphenidyl hydrochloride), cogentin (benztropine mesylate), akineton (biperiden hydrochloride), disipal (Norflex orphenadrine citrate), diphenhydramine, hydroxyzine, hyoscyamine, and Kemadrin (procyclidine hydrochloride).

In some embodiments, the anti-emetic agent is a steroid (e.g., a corticosteroid). Non-limiting examples of steroids include betamethasone, dexamethasone, methylprednisolone, Prednisone®, and trimethobenzamide (Tigan).

In some embodiments, the anti-emetic agent is an NK1-receptor antagonists (e.g., Neurokinin 1 substance P receptor antagonists). Non-limiting examples of NK1-receptor antagonists include aprepitant, casopitant, ezlopitant, fosaprepitant, maropitant, netupitant, rolapitant, and vestipitant.

Other non-limiting examples of NK1-receptor antagonists include: MPC-4505, GW597599, MPC-4505, GR205171, L-759274, SR 140333, CP-96,345, BIIF 1149, NKP 608C, NKP 608A, CGP 60829, SR 140333 (Nolpitantium besilate/chloride), LY 303870 (Lanepitant), MDL-105172A, MDL-103896, MEN-11149, MEN-11467, DNK 333A, YM-49244, YM-44778, ZM-274773, MEN-10930, S-19752, Neuronorm, YM-35375, DA-5018, MK-869, L-754030, CJ-11974, L-758298, DNK-33A, 6b-1, CJ-11974 j. Benserazide and carbidopak. TAK-637 [(aR,9R)-7-[3,5-bis(trifluoromethyl)benzyl]-8,9,10,11-tetrahydro-9-methyl-5-(4-methylphenyl)-7H-[1,4]diazocino[2,1-g] [1,7] naphthyridine-6,13-dione], PD 154075, ([(2-benzofuran)-CH2OCO]—(R)-alpha-MeTrp-(S)—NHCH(CH3) Ph), FK888, and (D-Pro4, D-Trp7,9,10, Phe 11)SP4-11.

In some embodiments, the anti-emetic agent is an anti-dopaminergic agents/dopamine receptor antagonist (e.g., dopamine receptor antagonist, e.g., D2 or D3 antagonists). Non-limiting examples include phenothiazines (e.g., promethazine, chlorpromazine, prochlorperazine, perphenazine, hydroxyzine, thiethylperazine, metopimazine,); benzamides (e.g., metoclopramide, domperidone), butyrophenones (e.g., haloperidol, droperidol); alizapride, bromopride, clebopride, domperidone, itopride, metoclopramide, trimethobenzamide, and amisulpride.

In some embodiments, the anti-emetic agent is a non-psychoactive cannabinoids (e.g., cannabidiol (CBD), cannabidiol dimethylheptyl (CBD-DMH), tetra-hydro-cannabinol (THC), cannabinoid agonists such as WIN 55-212 (a CB1 and CB2 receptor agonist), dronabinol (Marinol®), and nabilone (Cesamet)).

Other exemplary anti-emetic agents include: c-9280 (Merck); benzodiazepines (diazepam, midazolam, lorazepam); neuroleptics/anti-psychotics (e.g., dixyrazine, haloperidol, and Prochlorperazine (Compazine®)); cerium oxalate; propofol; sodium citrate; dextrose; fructose (Nauzene); orthophosphoric acid; fructose; glucose (Emetrol); bismuth subsalicylate (Pepto Bismol); ephedrine; vitamin B6; peppermint, lavender, and lemon essential oils; and ginger.

Still other exemplary anti-emetic agents include those disclosed in US 20120101089A1; U.S. Pat. No. 10,071,088 B2; U.S. Pat. No. 6,673,792 B1; U.S. Pat. No. 6,197,329 B1; U.S. Pat. No. 10,828,297 B2; U.S. Pat. No. 10,322,106 B2; U.S. Pat. No. 10,525,033 B2; WO 2009080351 A1; WO 2019203753 A2; WO 2002020001 A2; U.S. Pat. No. 8,119,697 B2; U.S. Pat. No. 5,039,528; US20090305964A1; and WO 2006/111169, each of which is incorporated by reference in its entirety.

In some embodiments, the anti-emetic agent is a 5HT3-receptor antagonist (serotonin receptor antagonist), neuroleptic agent, anti-psychotic agent, antihistamine, anticholinergic agent, steroid (e.g., corticosteroid), NK1-receptor antagonist (e.g., Neurokinin 1 substance P receptor antagonist), antidopaminergic agent/dopamine receptor antagonist, benzodiazepine, or cannabinoid (e.g., non-psychoactive cannabinoid).

In some embodiments, the anti-emetic agent is selected from the group consisting of granisetron, dolasetron, ondansetron, ondansetron hydrochloride, tropisetron, ramosteron, palonosetron, alosetron, azasetron, bemesetron, zatisetron, batanopirde, MDL-73147EF; Metoclopramide, N-3389, Y-25130 hydrochloride, MDL 72222, tropanyl-3,5-dimethylbenzoate, 3-(4-allylpiperazin-1-yl)-2-quinoxalinecarbonitrile maleate, zacopride hydrochloride, mirtazepine, cilansetron, clozapine, cyproheptadine, dazopride, hydroxyzine, lerisetron, mianserin, olanzapine, palonosetron (+netupitant), quetiapine, qamosetron, ramosteron, ricasetron, risperidone, ziprasidone, zatosetron, cyclizine, meclizine, meclizine hydrochloride, cinnarizine, promethazine, dimenhydrinate, diphenhydramine, buclizine, doxylamine, mirtazapine, atropine, scopolamine, glycopyrron, hyoscine, artane, benztropine mesylate, biperiden hydrochloride, Norflex orphenadrine citrate, diphenhydramine, hyoscyamine, procyclidine hydrochloride, betamethasone, dexamethasone, methylprednisolone, Prednisone®, trimethobenzamide (Tigan), aprepitant, casopitant, ezlopitant, fosaprepitant, maropitant, netupitant, rolapitant, and vestipitant, MPC-4505, GW597599, MPC-4505, GR205171, L-759274, SR 140333, CP-96,345, BIIF 1149, NKP 608C, NKP 608A, CGP 60829, SR 140333 (Nolpitantium besilate/chloride), LY 303870 (Lanepitant), MDL-105172A, MDL-103896, MEN-11149, MEN-11467, DNK 333A, YM-49244, YM-44778, ZM-274773, MEN-10930, S-19752, Neuronorm, YM-35375, DA-5018, MK-869, L-754030, CJ-11974, L-758298, DNK-33A, 6b-1, CJ-11974, Benserazide, carbidopa, TAK-637, PD 154075, FK888, (D-Pro4, D-Trp7,9,10, Phe11)SP4-11, chlorpromazine, prochlorperazine (Compazine®), perphenazine, thiethylperazine, metopimazine, domperidone, haloperidol, droperidol, alizapride, bromopride, clebopride, itopride, amisulpride, cannabidiol (CBD), cannabidiol dimethylheptyl (CBD-DMH), tetra-hydro-cannabinol (THC), WIN 55-212 (a CB1 and CB2 receptor agonist), dronabinol (Marinol®), nabilone (Cesamet)), c-9280, diazepam, midazolam, lorazepam, dixyrazine, cerium oxalate, propofol, sodium citrate, dextrose, fructose (Nauzene), orthophosphoric acid, glucose (Emetrol), bismuth subsalicylate, ephedrine, vitamin B6, peppermint, lavender, lemon essential oil, and ginger.

In some embodiments, the additional therapeutic agent or regimen is administered to the patient at about the same time as contacting with or administering the compounds and pharmaceutical compositions. By way of example, the additional therapeutic agent or regimen and the compounds and pharmaceutical compositions are provided to the patient simultaneously in the same dosage form.

In certain embodiments, provided is a fixed dose combination comprising an anti-emetic agent and a compound as described herein, or pharmaceutically acceptable salt or solvate thereof, or a compound disclosed herein, or pharmaceutically acceptable salt or solvate thereof.

As another example, the additional therapeutic agent or regimen and the compounds and pharmaceutical compositions are provided to the patient concurrently in separate dosage forms.

In some embodiments, the additional therapeutic agent or regimen is administered to the patient subsequently to, or after, contacting with or administering the compounds and pharmaceutical compositions (e.g., about one hour after, or about 6 hours after, or about 12 hours after, or about 24 hours after, or about 48 hours after).

In certain embodiments, the anti-emetic agent is a 5HT3-receptor antagonist (serotonin receptor antagonist). In certain embodiments, the anti-emetic agent is ondansetron. In certain embodiments, the anti-emetic agent is a dopamine receptor antagonist. In certain embodiments, the anti-emetic agent is a benzamide. In certain embodiments, the anti-emetic agent is metoclopramide.

### EXAMPLES

### Example 1: Synthesis of 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one (Compound I)

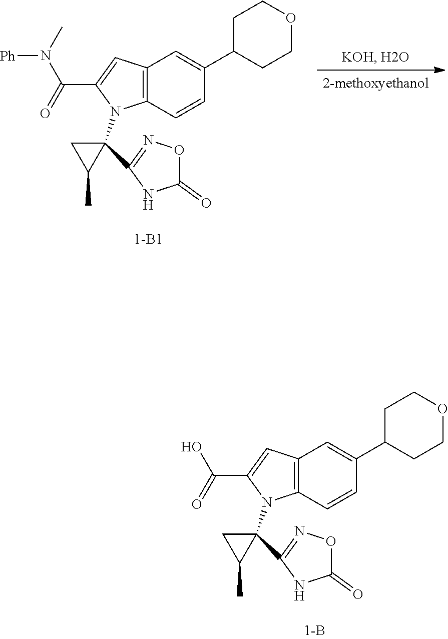 [SMILES: C[C@H]1C[C@]1(c1noc(=O)[nH]1)n1c(C(=O)N(C)c2ccccc2)cc2cc(C3CCOCC3)ccc21 ; C[C@H]1C[C@]1(c1noc(=O)[nH]1)n1c(C(=O)O)cc2cc(C3CCOCC3)ccc21]

To a solution of compound 1-B1 (1 eq.) in 2-methoxyethanol (4 vol) and H₂O (10.00 eq) was added KOH (10.00 eq), the mixture was heated at 100-105° C. for 12 hrs. After reaction was completed, the reaction mixture was cooled to 10-20° C., water (4 vol) was added in portions. The resulting mixture was poured into HCl (aq. 2M, 20 vol). Solid was formed and then the mixture was filtered. The filter cake was slurry with ACN (4 Vol) at 80-85° C. for 8 hrs. Then the mixture was filtered, the filter cake was collected and dried under vacuum. Compound 1-B was obtained as an off-white solid.

LC-MS: m/z 384.2 (M+H)⁺.

¹H NMR (400 MHz, DMSO-d₆) δ: 12.85 (s, 1H), 12.01-12.27 (m, 1H), 7.54 (d, J=4.8 Hz, 1H), 7.27-7.40 (m, 2H), 7.19 (d, J=4.0 Hz, 1H), 3.95-3.98 (m, 2H), 3.45-3.47 (m, 2H), 2.83-2.86 (m, 1H), 1.89-2.08 (m, 1H), 1.55-1.80 (m, 6H), 1.26-1.41 (m, 3H).

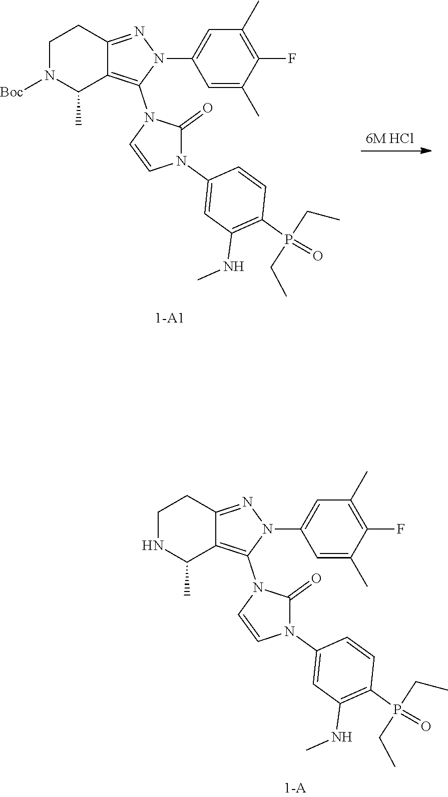 [SMILES: CCP(=O)(CC)c1ccc(-n2ccn(-c3c4c(nn3-c3cc(C)c(F)c(C)c3)CCN(C(=O)OC(C)(C)C)[C@H]4C)c2=O)cc1NC ; CCP(=O)(CC)c1ccc(-n2ccn(-c3c4c(nn3-c3cc(C)c(F)c(C)c3)CCN[C@H]4C)c2=O)cc1NC]

To a 2-MeTHF (2-3 Vol) solution of 1-A1 (1.0 eq.), 6 M HCl (13.5 eq.) was slowly added below 25° C. for about 3 h. After stirring for 10 h at 15-25° C. After reaction completely, 5 V H2O and 5 V Ethyl acetate were added into the reaction. Separation of the two phases was observed. Adjust pH of aqueous phase to 7-8 with 18% NaOH. pH of aqueous phase was further adjusted to 9˜10 with 5% NaOH aqueous solution, solid in aqueous phase was filtrated. After drying, Compound 1-A was obtained as an off-white solid.

LC-MS: m/z 551.36 (M+H)+.

¹H NMR (400 MHz, DMSO-d₆) δ: 7.72 (q, J=4.8 Hz, 1H), 7.35 (d, J=2.8 Hz, 1H), 7.26 (dd, J=8.0, 12.8 Hz, 1H), 7.11 (d, J=4.8 Hz, 2H), 6.85-6.93 (m, 3H), 3.94-3.97 (m, 1H), 3.21-3.24 (m, 1H), 2.86-2.91 (m, 1H), 2.73 (d, J=4.8 Hz, 3H), 2.65-2.69 (m, 2H), 2.19 (d, J=1.2 Hz, 6H), 1.88-1.97 (m, 4H), 1.11 (d, J=6.0 Hz, 3H), 0.95-1.04 (m, 6H).

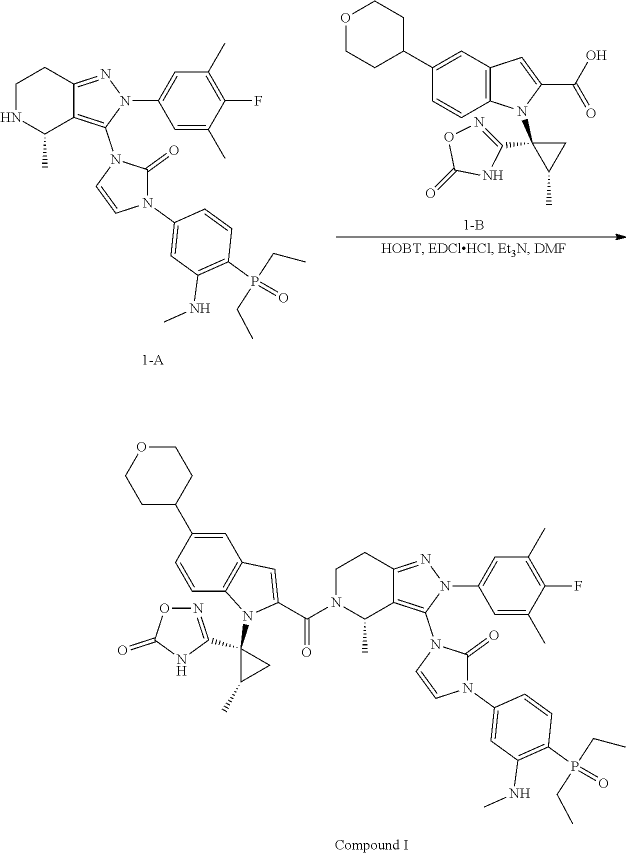 [SMILES: CCP(=O)(CC)c1ccc(-n2ccn(-c3c4c(nn3-c3cc(C)c(F)c(C)c3)CCN[C@H]4C)c2=O)cc1NC ; C[C@H]1C[C@]1(c1noc(=O)[nH]1)n1c(C(=O)O)cc2cc(C3CCOCC3)ccc21 ; "HOBT, EDCl·HCl, Et3N, DMF" ; CCP(=O)(CC)c1ccc(-n2ccn(-c3c4c(nn3-c3cc(C)c(F)c(C)c3)CCN(C(=O)c3cc5cc(C6CCOCC6)ccc5n3[C@@]3(c5noc(=O)[nH]5)C[C@@H]3C)[C@H]4C)c2=O)cc1NC]

Compound I was prepared from 1-A. A mixture of 1.10 eq. 1-B, 1.5 eq. EDCI and 1.6 eq. HOBt in 8 V DMF was stirred at room temperature for 1 hr. A solution of 1-A and TEA (4.0 eq.) in 7 V DMF was added into the solution of 1-B in DMF. After stirring at room temperature for 8 hr, the product was obtained. 30V H₂O was added to the reaction. The reaction mixture was extracted with DCM twice (10V and 5V). The combined DCM solution was washed with 15 V 7% NaHCO₃ solution. The DCM solution was washed with 5V H₂O again. Then, the organic phase was switched to 10 V EtOH. ˜1.5V ethyl acetate was added to the mixture. The precipitated solid was collected by filtration. Finally, the product was washed with EtOH and dried under vacuum at 40-50° C. for 16-24 h.

LC-MS: m/z 916.4 (M+H)+.

¹HNMR (400 MHz, DMSO-d₆, 80° C.) δ: 11.58 (br. s, 1H), 7.66 (br. s, 1H), 7.52 (s, 1H), 7.42 (d, J=8.4 Hz, 1H), 7.05-7.30 (m, 5H), 6.70-6.95 (m, 4H), 5.56 (br. s, 1H), 4.45 (br. s, 1H), 3.95-3.99 (m, 2H), 3.40-3.70 (m, 3H), 2.83-2.90 (m, 3H), 2.60-2.80 (m, 3H), 2.22 (d, J=1.6 Hz, 6H), 1.88-1.96 (m, 4H), 1.58-1.80 (m, 7H), 1.43 (br. s, 3H), 1.17 (br. s, 3H), 0.95-1.10 (m, 6H).

### Example 2: Synthesis of Compound I L-Arginine Salt

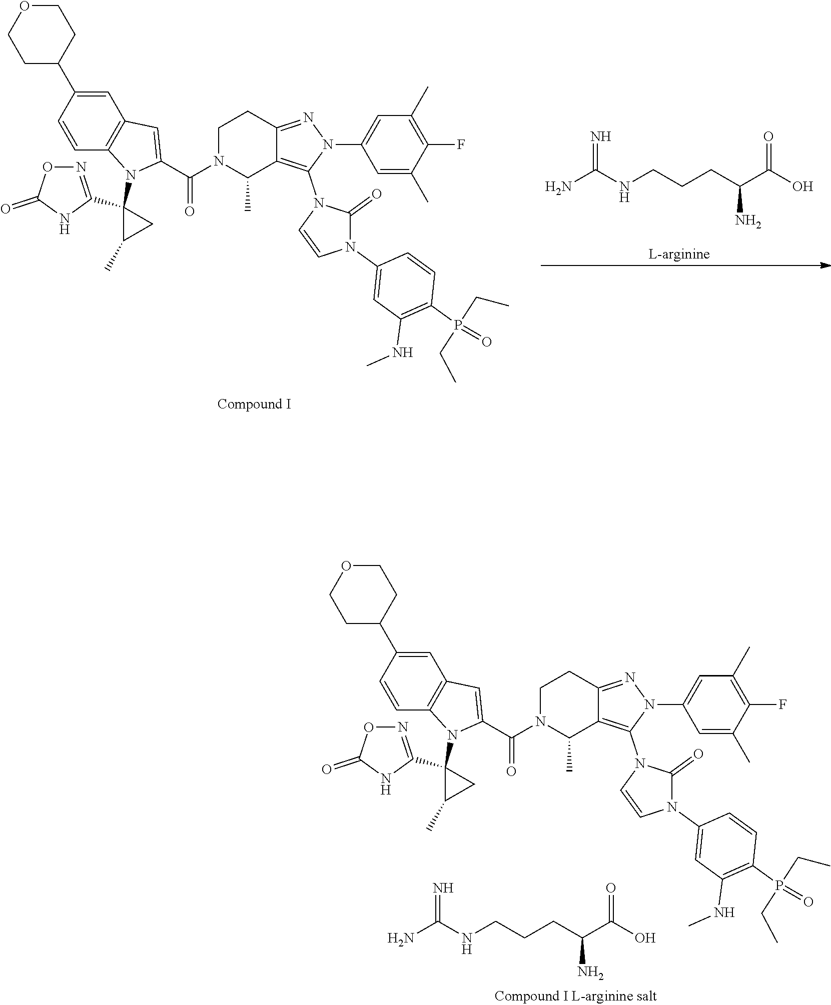 [SMILES: CCP(=O)(CC)c1ccc(-n2ccn(-c3c4c(nn3-c3cc(C)c(F)c(C)c3)CCN(C(=O)c3cc5cc(C6CCOCC6)ccc5n3[C@@]3(c5noc(=O)[nH]5)C[C@@H]3C)[C@H]4C)c2=O)cc1NC ; N=C(N)NCCC[C@H](N)C(=O)O ; CCP(=O)(CC)c1ccc(-n2ccn(-c3c4c(nn3-c3cc(C)c(F)c(C)c3)CCN(C(=O)c3cc5cc(C6CCOCC6)ccc5n3[C@@]3(c5noc(=O)[nH]5)C[C@@H]3C)[C@H]4C)c2=O)cc1NC ; N=C(N)NCCC[C@H](N)C(=O)O]

The Compound I L-arginine salt was prepared from the reaction of Compound I (1.0 eq.) and L-arginine (1.1 eq.) in IPA/H₂O (7:3 v/v). After stirring for ˜2 h at 50-55° C., 2.38 equivalents by volume IPA and 2 wt % crystal seed were added into the clear solution whereupon the mixture became hazy. After stirring for 2 h, ˜15.5 equivalents by volume IPA was added dropwise into the mixture. After stirring at 50-55° C. for 8 h, the mixture was further stirred at 0-5° C. for 10 h. The precipitated solid was collected by filtration, washed with IPA, and dried at 65-75° C. to give Compound I L-arginine salt.

HR-MS: m/z 916.4048 [M+H]⁺

¹H NMR (400 MHz, DMSO-d₆) δ: 7.71-7.73 (m, 1H), 7.37-7.65 (m, 2H), 7.24-7.31 (m, 1H), 7.00-7.21 (m, 5H), 6.72-6.94 (m, 2H), 6.74 (br. s, 1H), 5.62 (br. s, 1H), 4.70-4.81 (m, 1H), 3.78-3.98 (m, 2H), 3.28-3.46 (m, 4H), 3.01-3.11 (m, 2H), 2.66-2.90 (m, 4H), 2.16-2.20 (m, 6H), 1.88-1.95 (m, 4H), 1.55-1.74 (m, 10H), 1.13-1.35 (m, 6H), 0.94-1.05 (m, 6H).

### Example 3: Polymorphs of Compound I Free Acid

The polymorphs of Compound I free acids were investigated using the free acid (Compound I free acid Form A) starting material, which was prepared using the procedure described in Example 1.

### Approximate Solubility at 25° C. and at 50° C.

About 5 mg of Compound I free acid Form A was weighed to a 2 mL glass vial. 20 μL aliquots of each solvent were added to dissolve the drug substance at 25° C. Vortex and sonication were applied to assist dissolution. Max. volume of each solvent added was 1 mL. Approximate solubility was determined by visual observation.

About 10 mg of Compound I free acid Form A was weighed to a 2 mL glass vial. 20 μL aliquots of each solvent were added to dissolve the drug substance at 50° C. Vortex and sonication were applied to assist dissolution. Max. volume of each solvent added was 1 mL. Approximate solubility was determined by visual observation. The results of both solubility experiments are summarized in Table 3-1.

TABLE 3-1
Summary of approximate solubility at 25° C. and at 50° C.

| | 25° C. | | 50° C. | |
|---|---|---|---|---|
| | Solubility | | Solubility | |
| Solvents | (mg/mL) | Comments | (mg/mL) | Comments |
| Water | <5 | — | <5 | — |
| Methanol | <5 | Decreased | <5 | Decreased |
| | | solids amount | | solids |
| | | | | amount |
| Ethanol | <5 | — | <5 | — |
| Acetone | 63-83 | — | 100-125 | — |
| Acetonitrile | 63-83 | — | >500 | — |
| Tetrahydrofuran | >250 | — | >500 | — |
| 2-MeTHF | 125-250 | — | 250-500 | — |
| 1,4-Dioxane | 125-250 | — | 250-500 | — |
| Ethyl acetate | <5 | Decreased | <5 | Decreased |
| | | solids amount | | solids |
| | | | | amount |
| Isopropyl acetate | ~5 | Almost clear | 5-10 | — |
| | | solution | | |
| MTBE | <5 | Decreased | 5-10 | — |
| | | solids amount | | |
| Heptane | <5 | — | <5 | — |
| Dichloromethane | 125-250 | — | // | — |
| DMSO | 125-250 | — | >250 | — |
| Toluene | 13-17 | — | 20-25 | — |
| IPA | <5 | Placed at 50° C., | 5-10 | — |
| | | clear solution | | |

//: Not tested

### Polymorph Screening of Compound I Free Acid Form A

### Preparation of Compound I Free Acid Form A

Compound I free acid Form A was prepared using the procedure described in example 1. About 7.5 g of the final product was weighed and dried under-vacuum at 60° C. for about 8 h. ¹H-NMR showed about 0.4 EtOH residue by weight (about 0.08 equiv. by molar ratio). The dried free acid Form A was used in the subsequent polymorph screening experiments in this section.

Equilibration with Solvents at 25° C. for 2 Weeks

Based on approximate solubility results, about 50 mg of Compound I free acid Form A was equilibrated in 0.2-1 mL of solvents at 25° C. for 2 weeks with a stirring bar on a magnetic stirring plate at a rate of 300-400 rpm. Obtained suspensions were filtered through a 0.45 μm nylon membrane filter by centrifugation at 14,000 rpm. Solid parts (wet cakes) were investigated by XRPD. For samples with different XRPD patterns, additional analysis including DSC, TGA, ¹H-NMR, and KF for hydrate were performed. The results are summarized in Table 3-2.

TABLE 3-2
Summary of equilibration with solvents at 25° C. for 2 weeks

| Solvents | XRPD | Additional analysis | Comments |
|---|---|---|---|
| MeOH | Form A | // | — |
| EtOH | Form A | // | — |
| Ethyl acetate | Form A | // | — |
| Isopropyl acetate | Form A | // | — |
| Acetone | Form A | // | — |
| ACN | Form F | // | — |
| MTBE | Form C | // | — |
| Toluene | Form A | // | — |
| 2-MeTHF | Form A | // | Few solids |
| 1,4-Dioxane | Form A | // | Few solids |
| MeOH/THF(v:v = 1:1) | Form A | // | — |
| THF/Heptane | Form A | // | — |
| (v:v = 1:1) | | | |
| Acetone/water | Form H | DSC: Dehydration | — |
| (v:v = 1:1) | | from about 10° C. | |
| a.w. = 0.85 | | Endothermic peak | |
| | | from about 75° C. | |
| | | Melting onset: 208.6° C. | |
| | | Enthalpy: 24 J/g | |
| | | TGA: 7.7% @ 180° C. | |
| | | ¹H-NMR: No detectable | |
| | | residual solvent | |
| | | KF: 9.6% (equal to | |
| | | ~5.5 equiv. water) | |
| ACN/water(v:v = 9:1) | Form D | // | — |
| a.w. = 0.87 | | | |
| THF/water(v:v = 9:1) | Form E | // | — |
| a.w. = 0.71 | | | |
| DMSO/water(v:v = 1:1) | Similar to | // | — |
| a.w. = 0.68 | Form G, Low | | |
| | crystallinity | | |
| Water | Form A + | // | / |
| | Form C | | |
| ACN/water(v:v = 9:1) | Form N | DSC: Dehydration | |
| a.w. = 0.87 | | from about 8° C. | |
| | | Endothermic peak | |
| | | onset: 75.9° C. | |
| | | Melting onset: 200.7° C. | |
| | | Enthalpy: 17 J/g | |
| | | TGA: About 9.0% @ 200° C. | |
| | | ¹H-NMR: No detectable | |
| | | residual solvent | |
| | | KF: 10.3% (equal to | |
| | | about 5.8 equiv. water) | |
| IPA | Form A | // | — |

//: Not tested

Equilibration with Solvents at 50° C. for 1 Week

Based on approximate solubility results, about 60 mg of Compound I free acid Form A was equilibrated in 0.2-1 mL of solvents at 50° C. for 1 week with a stirring bar on a magnetic stirring plate at a rate of 300-400 rpm. Obtained suspensions were filtered through a 0.45 μm nylon membrane filter by centrifugation at 14,000 rpm. Solid parts (wet cakes) were investigated by XRPD. For samples with different XRPD patterns, additional analysis including DSC, TGA, ¹H-NMR, and KF for hydrate were performed. The results are summarized in Table 3-3.

TABLE 3-3
Summary of equilibration with solvents at 50° C. for 1 week

| Solvents | XRPD | Additional analysis | Comments |
|---|---|---|---|
| MeOH | Form A | // | — |
| EtOH | Form A | // | — |
| Ethyl acetate | Form A | // | — |
| Isopropyl acetate | Form A | // | — |
| Acetone | Form A | // | — |
| ACN | Form F | DSC: Dehydration | — |
| | | from about 5° C. | |
| | | Endothermic peak | |
| | | onset: 117.4° C. | |
| | | Endothermic peak | |
| | | onset: 153.7° C. | |
| | | Melting onset: 218.6° C. | |
| | | Enthalpy: 35 J/g | |
| | | TGA: 1.2% @ 200° C. | |
| | | ¹H-NMR: No detectable | |
| | | residual solvent | |
| | | KF: 1.7% (equal to | |
| | | ~0.9 equiv. water) | |
| MTBE | Form C | // | — |
| Toluene | Form A | // | — |
| 2-MeTHF | // | // | Gel |
| 1,4-Dioxane | Form A | // | — |
| MeOH/THF | Form A | // | — |
| (v:v = 1:1) | | | |
| THF/Heptane | Form A | // | — |
| (v:v = 1:1) | | | |
| Acetone/water | Form A | // | — |
| (v:v = 1:1) | | | |
| a.w. = 0.85 | | | |
| ACN/water | // | // | Clear solution |
| (v:v = 9:1) | | | |
| a.w. = 0.82 | | | |
| THF/water | Form E | // | — |
| (v:v = 9:1) | | | |
| a.w. = 0.73 | | | |
| DMSO/water | Form G | DSC: Dehydration | — |
| (v:v = 1:1) | | from about 8° C. | |
| a.w. = 0.70 | | Melting onset: 201.1° C. | |
| | | Enthalpy: 22 J/g | |
| | | TGA: 8.5% @ 55° C. | |
| | | 5.9% @ 55-180° C. | |
| | | ¹H-NMR: 0.1 equiv. DMSO | |
| | | (About 0.7% by weight) | |
| | | KF: 14.1% (equal to | |
| | | ~8.4 equiv. water) | |
| Water | Form A + | // | — |
| | Form M | | |
| DMSO/water | Form L | DSC: Dehydration/desolvation | Melting upon |
| (v:v = 1:1) | | from about 6° C. | dehydration/desolvation |
| a.w. = 0.70 | | Melting upon | from about 120° C. |
| | | dehydration/desolvation | (Photos by melting |
| | | from about 120° C. | point apparatus) |
| | | TGA: 7.9% @ 70° C. | |
| | | 7.4% @ 70-200° C. | |
| | | ¹H-NMR: 1.8 equiv. DMSO | |
| | | (About 12.0% by weight) | |
| | | KF: 9.6% (equal to about | |
| | | 6.2 equiv. water) | |
| IPA | Form A | // | — |

//: Not tested

### Equilibration Under a Temperature Cycle

Based on approximate solubility results, about 50 mg of Compound I free acid Form A was equilibrated in 0.2˜1 mL of solvents under a temperature cycle between 5° C. to 50° C. at a heating/cooling rate of 0.1° C./min for 10 cycles. The equilibration was executed with a stirring bar on a magnetic stirring plate at a rate of 300-400 rpm. Obtained suspensions were filtered through a 0.45 μm nylon membrane filter by centrifugation at 14,000 rpm. Solid parts (wet cakes) were investigated by XRPD. For samples with different XRPD patterns, additional analysis including DSC, TGA, ¹H-NMR, and KF for hydrates were performed. The results are summarized in Table 3-4.

TABLE 3-4
Summary of equilibration under a temperature cycle

| | | Additional | |
|---|---|---|---|
| Solvents | XRPD | analysis | Comments |
| MeOH | Form A | // | — |
| EtOH | Form A | // | — |
| Ethyl acetate | Form A | // | — |
| Isopropyl acetate | Form A | // | — |
| Acetone | Form A | // | — |
| ACN | Form B | DSC: Dehydration | — |
| | | from about 4° C. | |
| | | Melting onset: 192.1° C. | |
| | | Enthalpy: 21 J/g | |
| | | TGA: 3.3% @ 180° C. | |
| | | ¹H-NMR: No detectable | |
| | | residual solvent | |
| | | KF: 4.6% (equal to | |
| | | ~2.5 equiv. water) | |
| MTBE | Form C | DSC: Dehydration | — |
| | | from about 5° C. | |
| | | Endothermic peak | |
| | | onset: 127.5° C. | |
| | | Melting onset: 190.3° C. | |
| | | Enthalpy: 22 J/g | |
| | | TGA: 5.5% @ 180° C. | |
| | | ¹H-NMR: 0.2% residual | |
| | | MTBE by weight | |
| | | (about 0.02 equiv. | |
| | | by molar ratio) | |
| | | KF: 5.1% (equal to | |
| | | ~2.7 equiv. water) | |
| Toluene | Form A | // | — |
| 2-MeTHF | // | // | Clear solution |
| 1,4-Dioxane | // | // | Clear solution |
| MeOH/THF | Form A | // | — |
| (v:v = 1:1) | | | |
| THF/Heptane | Form A | // | — |
| (v:v = 1:1) | | | |
| Acetone/water | Form A | // | — |
| (v:v = 1:1) | | | |
| ACN/water | Form D | DSC: Dehydration | — |
| (v:v = 9:1) | | from about 5° C. | |
| | | Exothermic peak | |
| | | onset: 132.0° C. | |
| | | Endothermic peak | |
| | | onset: 185.8° C. | |
| | | Melting onset: 215.0° C. | |
| | | Enthalpy: 29 J/g | |
| | | TGA: 1.4% @ 200° C. | |
| | | ¹H-NMR: No detectable | |
| | | residual solvent | |
| | | KF: 2.2% (equal to | |
| | | ~1.1 equiv. water) | |
| THF/water | Form E | DSC: Dehydration | Oil converted |
| (v:v = 1:1) | | from about 3° C. | to particles |
| | | Melting onset: 213.7° C. | |
| | | Enthalpy: 15 J/g | |
| | | TGA: 2.5% @ 200° C. | |
| | | ¹H-NMR: No detectable | |
| | | residual solvent | |
| | | KF: 10.3% (equal to | |
| | | ~5.8 equiv. water) | |
| DMSO/water | Form A | // | — |
| (v:v = 1:1) | | | |
| Water | Form M + | // | — |
| | Form A | | |

//: Not tested

Equilibration with Solvents at 25° C. for One Week with Amorphous Form

About 30 mg of Compound I free acid amorphous form, was equilibrated in 0.2-1 mL of solvents at 25° C. for 1 week with a stirring bar on a magnetic stirring plate at a rate of 300-400 rpm. Obtained suspensions were filtered through a 0.45 μm nylon membrane filter by centrifugation at 14,000 rpm. Solid parts (wet cakes) were investigated by XRPD. The results are summarized in Table 3-5.

TABLE 3-5
Summary of equilibration with solvents at
25° C. for one week with amorphous form

| Solvents | XRPD | Comments |
|---|---|---|
| EtOH | Form A | — |
| IPA | Form A | — |
| Acetone | Almost amorphous form | — |
| EA | Form A | — |
| MTBE | Form C | — |
| MEK | Form A | — |
| Water | Form M | Low crystallinity. |

### Crystallization at Room Temperature by Slow Evaporation

Based on approximate solubility results, about 20 mg of Compound I free acid Form A was dissolved in ˜1 mL of solvents. Obtained solutions were filtered through a 0.45 μm syringe nylon membrane filter. The clear solutions were slowly evaporated in ambient condition (about 20-25° C., 30-70% RH). Solid residues were investigated by XRPD. The results are summarized in Table 3-6.

TABLE 3-6
Summary of crystallization at room
temperature by slow evaporation

| Solvents | XRPD | Comments |
|---|---|---|
| Acetone | // | Sticky material |
| Acetonitrile | Amorphous form | — |
| Tetrahydrofuran | Amorphous form | — |
| 2-MeTHF | Amorphous form | — |
| IPAc | Form A + Form H | — |
| Dichloromethane | Amorphous form | — |
| Toluene | Amorphous form | — |

//: Not tested

### Crystallization at Room Temperature by Fast Evaporation

Based on approximate solubility results, about 20 mg of Compound I free acid Form A was dissolved in 0.2-1 mL of solvents. Obtained solutions were filtered through a 0.45 μm syringe membrane filter. The clear solutions were fast evaporated at room temperature (about 20-25° C.) under a dry nitrogen flow. Solid residues were investigated by XRPD. The results are summarized in Table 3-7.

TABLE 3-7
Summary of crystallization at room temperature by fast evaporation

| Solvents | XRPD |
|---|---|
| Acetone | Amorphous form |
| Acetonitrile | Amorphous form |
| Tetrahydrofuran | Amorphous form |
| 2-MeTHF | Amorphous form |
| IPAc | Amorphous form |
| Dichloromethane | Amorphous form |
| Toluene | Amorphous form |

Crystallization from Hot Saturated Solutions by Slow Cooling

Based on approximate solubility results, about 30 mg of Compound I free acid Form A was dissolved in the minimal amount of selected solvents at 50° C. Obtained solutions were filtered through a 0.45 m syringe membrane filter. The clear solutions were cooled to 5° C. at 0.1° C./min. Samples without precipitates at 5° C. were further cooled to −20° C. Precipitates were collected by centrifugation filtration through a 0.45 μm nylon membrane filter at 14,000 rpm. Solid parts (wet cakes) were investigated by XRPD. The results are summarized in Table 3-8.

TABLE 3-8
Summary of crystallization from hot
saturated solutions by slow cooling

| Solvents | XRPD | Additional analysis | Comments |
|---|---|---|---|
| Acetone | // | Clear solution | — |
| | | @ −20° C. | |
| Acetonitrile | Form B | // | — |
| IPAc | Form A | // | — |
| Toluene | Form A | // | — |
| THF/water(v:v = 1:1) | // | // | Oil |
| THF/MTBE (v:v = 2:1) | // | Clear solution | — |
| | | @ −20° C. | |
| 2-MeTHF/Heptane | Form A | // | — |
| (v:v = 2:1) | | | |

//: Not tested

Crystallization from Hot Saturated Solutions by Fast Cooling

Based on approximate solubility results, about 30 mg of Compound I free acid Form A was dissolved in the minimal amount of selected solvents at 50° C. Obtained solutions were filtered through a 0.45 μm syringe membrane filter. The clear solutions were put into a 5° C. stirring plate and agitated. Precipitates were collected by centrifugation filtration through a 0.45 μm nylon membrane filter at 14,000 rpm. Solid parts (wet cakes) were investigated by XRPD. The results are summarized in Table 3-9.

TABLE 3-9
Summary of crystallization from hot
saturated solutions by fast cooling

| Solvents | XRPD | Additional analysis | Comments |
|---|---|---|---|
| Acetone | // | Clear solution | — |
| | | @ −20° C. | |
| Acetonitrile | Form F | // | — |
| IPAc | // | Clear solution | — |
| | | @ −20° C. | |
| Toluene | Form A | // | — |
| THF/water(v:v = 1:1) | // | // | Oil |
| THF/MTBE (v:v = 2:1) | // | Clear solution | — |
| | | @ −20° C. | |
| 2-MeTHF/Heptane | Form A | // | — |
| (v:v = 2:1) | | | |

//: Not tested

### Crystallization by Addition of Anti-Solvent

Based on approximate solubility results, about 40 mg of Compound I free acid Form A was dissolved in the minimal amount of selected good solvents at ambient temperature (about 20-25° C.). Obtained solutions were filtered through a 0.45 μm syringe membrane filter. 4-8 folds of anti-solvent were added into the clear solutions slowly until a large amount of solids precipitated out. Precipitates were collected by centrifugation filtration through a 0.45 μm nylon membrane filter at 14,000 rpm. Solid parts (wet cakes) were investigated by XRPD. The results are summarized in Table 3-10.

TABLE 3-10
Summary of crystallization by addition of anti-solvent

| | Anti-solvent | | Additional | |
|---|---|---|---|---|
| Solvents (mL) | (mL) | XRPD | analysis | Comments |
| Acetone (1) | Heptane (2.5) | Form A | ¹H-NMR: No detectable | Suspension → oil → |
| | | | residual solvent | suspension |
| ACN (0.5) | EtOAc (2) | Form A | // | Clear solution→ |
| | | | | suspension |
| ACN (0.5) | EtOH (2) | Form A | ¹H-NMR: No detectable | Clear solution → |
| | | | residual solvent | suspension |
| THF (0.15) | EtOH (0.6) | Form A | ¹H-NMR: No detectable | Clear solution → |
| | | | residual solvent | suspension |
| 2-MeTHF | EA (1) | Form A | ¹H-NMR: No detectable | Clear solution → |
| (0.25) | | | residual solvent | suspension |
| 1,4-Dioxane | Water (0.35) | Form E | After equilibrated | Suspension→ sticky |
| (0.35) | | | for 10 days: Form H | material →suspension |
| DCM (0.2) | Heptane (0.4) | Form A | // | Suspension→ sticky |
| | | | | material → suspension |
| Toluene (3) | Heptane (12) | Amorphous | After equilibrated | Suspension |
| | | form | for 10 days: | |
| | | | Amorphous form | |

//: Not tested

### Crystallization by Reverse Addition of Anti-Solvent

Based on approximate solubility results, about 40 mg of Compound I free acid Form A was dissolved in the minimal amount of selected good solvents at ambient temperature (about 20-25° C.). Obtained solutions were filtered through a 0.45 m syringe membrane filter. The clear solutions were added into 4-8 folds of anti-solvent quickly. Precipitates were collected by centrifugation filtration through a 0.45 Cm nylon membrane filter at 14,000 rpm. Solid parts (wet cakes) were investigated by XRPD. For samples with different XRPD patterns, additional analysis including DSC, TGA, ¹H-NMR, and KF for hydrate were performed. The results are summarized in Table 3-11.

TABLE 3-11
Summary of crystallization by reverse addition of anti-solvent

| Solvents | Anti-solvent | | | |
|---|---|---|---|---|
| (mL) | (mL) | XRPD | Additional analysis | Comments |
| Acetone (1) | Heptane (4) | Form A | ¹H-NMR: No | Suspension → oil → |
| | | | detectable residual | suspension |
| | | | solvent | |
| ACN (0.5) | EtOAc (2) | Form A | // | Clear solution → |
| | | | | suspension |
| ACN (0.5) | EtOH (2) | // | // | Clear solution, |
| | | | | evaporated in fume |
| | | | | hood: Form A |
| THF (0.15) | EtOH (0.6) | Form A | ¹H-NMR: No | Clear solution → |
| | | | detectable residual | suspension |
| | | | solvent | |
| 2-MeTHF | EtOAc (1) | Pattern A | ¹H-NMR: No | Clear solution → |
| (0.25) | | | detectable residual | suspension |
| | | | solvent | |
| 1,4-Dioxane | Water (1.4) | Amorphous | After equilibrating | Suspension |
| (0.35) | | form | for 10 days: Form I | |
| | | | DSC: Dehydration | |
| | | | from about 13° C. | |
| | | | Melting onset: | |
| | | | 198.9° C. | |
| | | | Enthalpy: 9 J/g | |
| | | | TGA: 7.4% @ | |
| | | | 200° C. | |
| | | | ¹H-NMR: No | |
| | | | detectable residual | |
| | | | solvent | |
| DCM (0.2) | Heptane (0.8) | Form A | // | Suspension → |
| | | | | sticky material → |
| | | | | suspension |
| Toluene (3) | Heptane (12) | Amorphous | After equilibrating | Suspension |
| | | form | for 10 days: | |
| | | | Amorphous form | |

//: Not tested

### Crystallization by Vapor Diffusion

Based on approximate solubility results, about 30 mg of Compound I free acid Form A was dissolved in the minimal amount of selected solvents at ambient temperature (about 20-25° C.). Obtained solutions were filtered through a 0.45 μm syringe membrane filter. The clear solutions were transferred into 4 mL glass vials without lids. Then these 4 mL lid less vials were placed to 40 mL glass vials. To the 40 mL vials were added anti-solvents. Then these 40 mL vials were capped tightly and placed at ambient temperature (about 20-25° C.) for up to 14 days. Precipitates were collected by centrifugation filtration through a 0.45 μm nylon membrane filter at 14,000 rpm. Solid parts (wet cakes) were investigated by XRPD. The results are summarized in Table 3-12.

TABLE 3-12
Summary of crystallization by vapor diffusion

| Solvents (μL) | Anti-solvent (μL) | XRPD | Comments |
|---|---|---|---|
| 2-MeTHF (200) | MeOH (800) | // | Clear solution, |
| | | | evaporated in fume |
| | | | hood: Form A |
| DMSO (100) | EtOH (400) | Form A | — |
| DMSO (100) | EtOAc (400) | // | Clear solution, |
| | | | evaporated in fume |
| | | | hood: almost |
| | | | amorphous form |
| 1,4-Dioxane (200) | EtOAc (800) | // | Clear solution, |
| | | | evaporated in fume |
| | | | hood: sticky material |

//: Not tested

### Crystallization by Heat-Cool DSC

Polymorphic behavior of Compound I free acid Form A was investigated by two different heat-cool DSC cycles. The results are summarized in Table 3-13.

TABLE 3-13
Summary of crystallization by heat-cool DSC

| Heat-cool cycles | Thermal events |
|---|---|
| Cycle 1 | Dehydration from about 7° C. |
| Step 1: 0° C. to 230° C. at 10° | Two combined endothermic peaks |
| C./min; | Tₒₙₛₑₜ: 202.6° C. |
| Step 2: 230° C. to −20° C. at | T_{g}: 179.9° C. |
| 20° C./min; | |
| Step 3: reheat to 250° C. at 10° | |
| C./min. | |
| Cycle 2 | Dehydration from about 12° C. |
| Step 1: 0° C. to 230° C. at 10° | Two combined endothermic peaks |
| C./min; | Tₒₙₛₑₜ: 202.2° C. |
| Step 2: 230° C. to −20° C. at | T_{g}: 179.7° C. |
| 2° C./min; | |
| Step 3: reheat to 250° C. at 10° | |
| C./min. | |

### Interrelationship Investigation of Polymorphs by Variable Temperature XRPD

The interrelationship between the Compound I free acid polymorphs was investigated by variable temperature XRPD (VT-XRPD). The results are summarized in Table 3-14.

TABLE 3-14
Summary of VT-XRPD experiments

| Initial | Temperature | | |
|---|---|---|---|
| Polymorph | (condition) | XRPD | Comments |
| Form A | 25° C. (initial) | Form A | — |
| | 120° C. (N₂) | Form A1 | — |
| | 25° C. (N₂) | Form A1 | — |
| | 25° C. | Form A | DSC: |
| | | | Dehydration |
| | | | from about |
| | | | 4° C. |
| Form C | 25° C. (initial) | Form C | Heat-cool |
| | 100° C. (N₂) | Form C1 | DSC of Form |
| | 150° C. (N₂) | Form C2 | C: Cycle 1: |
| | | | endothermic |
| | | | peak onset: |
| | | | 130.7° C. |
| | | | Cycle 2: |
| | | | exothermic |
| | | | peak onset: |
| | | | 107.3° C. |
| | 25° C. | Physical mixture | DSC: |
| | | of Form C and | Dehydration |
| | | Form C1 | from about |
| | | | 13° C. |
| Form F | 25° C. (initial) | Form F | — |
| | 90° C. (N₂) | Form F1 | |
| | 130° C. (N₂) | Form F1 | |
| | 180° C. (N₂) | Form F1 | |
| | 25° C. | Form F | |
| Form H | 25° C. (initial) | Form H | — |
| | 60° C. (N₂) | Form E1 | |
| | 150° C. (N₂) | Form E1 | |
| | 25° C. | Physical mixture | DSC: |
| | | of Form E and | dehydration |
| | | Form E1 | form about |
| | | | 12° C. |
| Form I | 25° C. (initial) | Form I | |
| | 120° C. (N₂) | Form I1 | |
| | 25° C. (N₂) | Form I | |
| Form J | 25° C. (initial) | Form J | — |
| | 150° C. (N₂) | Form D1 | |
| | 200° C. (N₂) | Form D1 | |
| | 25° C. | Form D | |
| Form K | 25° C. (initial) | Form K | — |
| | 100° C. (N₂) | Form B1 | |
| | 25° C. (N₂) | Form K | |
| Form N | 25° C. (initial) | Form N | — |
| | 75° C. (N₂) | Form N1 | |
| | 120° C. (N₂) | Form N1 | |
| | 25° C. (ambient | Form N | |
| | condition for about | | |
| | 20 h) | | |

### Water Activity Experiments

The water activity experiments were conducted at 25° C. in MeOH/water system to determine critical water activity among Compound I free acid Form A, Form C, Form E, Form F, Form H, Form I, Form J, Form K, Form M and Form N. About 1.5 mg of each sample was added to 0.2 mL saturated solutions of MeOH/water system. Obtained suspensions were stirred at 25° C. for 3 days, respectively. Solid parts (wet cakes) were isolated by centrifugation filtration and investigated by XRPD. The results are summarized in Table 3-15.

TABLE 3-15
Summary of water activity experiments

| | |
|---|---|
| | XRPD at 25° C. |
| Solvents | after 3 days |

Forms A, C, E, F, H, I

| | |
|---|---|
| MeOH (a.w. = 0)* | Form A |
| MeOH/water (v:v = 935:65) (a.w. = 0.2)* | Form A |
| MeOH/water (v:v = 840:160) (a.w. = 0.4)* | Form A |
| MeOH/water (v:v = 690:310) (a.w. = 0.6)* | Form A |
| MeOH/water (v:v = 420:580) (a.w. = 0.8)* | Form A |
| Water (a.w. = 1)* | Form M |

Form A, J, K, M, N

| | |
|---|---|
| MeOH(a.w. = 0)* | Form A |
| MeOH/water (v:v = 935:65) (a.w. = 0.2)* | Form A |
| MeOH/water (v:v = 840:160) (a.w. = 0.4)* | Form A |

Forms A, C, E, F, H, I

| | |
|---|---|
| MeOH/water (v:v = 690:310) (a.w. = 0.6)* | Form A |
| MeOH/water (v:v = 420:580) (a.w. = 0.8)* | Form A |
| Water (a.w. = 1)* | Form N |

Note:
water activity is calculated by UNIFAC method

### Conclusion

A total of 12 stable crystalline polymorphs of Compound I free acids were obtained from the screening experiments using free acid Form A as starting material. Free acid Form M is a metastable hydrate of low crystallinity and free acid Form L is a DMSO-water hetero solvate which is isostructural with free acid Form N.

### Free Acid Form A

Compound I free acid Form A was prepared by the procedure described in Example 1, via slurry of Compound I free acid in EtOAc at room temperature for 2 days. The XRPD of free acid Form A is shown in FIG. 4A. The DSC curve of free acid Form A is shown in FIG. 4B, which showed two endothermic peaks at 49.7° C. and 211.3° C. The TGA curve of free acid Form A is shown in FIG. 4C, which showed a weight loss of 2.3% up to 200° C.

### Free Acid Form B

Compound I free acid Form B was prepared by equilibration of free acid Form A in acetonitrile under a temperature cycle or via crystallization by slow cooling from a saturated solution of Compound I free acid Form A in ACN. The XRPD of free acid Form B is shown in FIG. 5A. The DSC curve of free acid Form B is shown in FIG. 5B, which showed two endothermic peaks at 32.4° C. and 199.0° C. The TGA curve of free acid Form B is shown in FIG. 5C, which showed a weight loss of 3.3% up to 180° C.

### Free Acid Form C

Compound I free acid Form C was prepared by equilibration of free acid Form A in MTBE. The XRPD of free acid Form C is shown in FIG. 6A. The DSC curve of free acid Form C is shown in FIG. 6B, which showed three endothermic peaks at 31.7° C., 134.9° C., and 194.7° C. The TGA curve of free acid Form C is shown in FIG. 6C, which showed a weight loss of 5.5% up to 180° C.

### Free Acid Form D

Compound I free acid Form D was prepared by equilibration of free acid Form A in ACN/water (9:1 v/v). The XRPD of free acid Form D is shown in FIG. 7A. The DSC curve of free acid Form D is shown in FIG. 7B, which showed three endothermic peaks at 36.1° C., 198.1° C., and 223.9° C., as well as an exothermic peak at 133.7° C. The TGA curve of free acid Form D is shown in FIG. 7C, which showed a weight loss of 1.4% up to 200° C.

### Free Acid Form E

Compound I free acid Form E was prepared by equilibration of free acid Form A in THF/water (9:1 v/v). The XRPD of free acid Form E is shown in FIG. 8A. The DSC curve of free acid Form E is shown in FIG. 8B, which showed two endothermic peaks at 43.6° C. and 223.9° C. The TGA curve of free acid Form E is shown in FIG. 8C, which showed a weight loss of 2.5% up to 200° C.

### Free Acid Form F

Compound I free acid Form F was prepared by equilibration of free acid Form A in acetonitrile. The XRPD of free acid Form F is shown in FIG. 9A. The DSC curve of free acid Form F is shown in FIG. 9B, which showed four endothermic peaks at 46.2° C., 121.0° C., 159.4° C., and 230.4° C. The TGA curve of free acid Form F is shown in FIG. 9C, which showed a weight loss of 1.2% up to 200° C.

### Free Acid Form G

Compound I free acid Form G was prepared by equilibration of free acid Form A in DMSO/water (1:1 v/v). The XRPD of free acid Form G is shown in FIG. 10A. The DSC curve of free acid Form G is shown in FIG. 10B, which showed two endothermic peaks at 52.9° C. and 208.7° C. The TGA curve of free acid Form G is shown in FIG. 10C, which showed a weight loss of 8.5% up to 55° C. and additionally 5.9% from 55-180° C.

### Free Acid Form H

Compound I free acid Form H was prepared by equilibration of free acid Form A in acetone/water (1:1 v/v) at 25° C. The XRPD of free acid Form H is shown in FIG. 11A. The DSC curve of free acid Form H is shown in FIG. 11B, which showed three endothermic peaks at 51.3° C., 105.5° C., and 217.2° C. The TGA curve of free acid Form H is shown in FIG. 11C, which showed a weight loss of 7.7% up to 180° C.

### Free Acid Form I

Compound I free acid Form I was prepared from free acid Form A in 1,4-dioxane by reverse addition of anti-solvent (water). The XRPD of free acid Form I is shown in FIG. 12A. The DSC curve of free acid Form I is shown in FIG. 12B, which showed two endothermic peaks at 41.5° C. and 207.3° C. The TGA curve of free acid Form I is shown in FIG. 12C, which showed a weight loss of 7.4% up to 200° C.

### Free Acid Form J

Compound I free acid Form J was obtained from storing free acid Form D at ambient conditions (23-27° C., 50-70% RH) over 2 weeks. The XRPD of free acid Form J is shown in FIG. 13A. The DSC curve of free acid Form J is shown in FIG. 13B, which showed two endothermic peaks at 68.6° C. and 221.2° C. The TGA curve of free acid Form J is shown in FIG. 13C, which showed a weight loss of 2.2% up to 200° C.

### Free Acid Form K

Compound I free acid Form K was obtained from storing free acid Form B at ambient conditions (23-27° C., 50-70% RH) over 2 weeks. The XRPD of free acid Form K is shown in FIG. 14A. The DSC curve of free acid Form K is shown in FIG. 14B, which showed two endothermic peaks at 46.1° C. and 198.4° C. The TGA curve of free acid Form K is shown in FIG. 14C, which showed a weight loss of 2.4% up to 70° C. and additionally 2.6% from 70-170° C.

### Free Acid Form L

Compound I free acid Form L is a DMSO-water hetero solvate. It is in isostructural with hydrate Form N. It was obtained from DMSO/water (v:v=1:1) by equilibration at 50° C. DSC shows a dehydration/desolvation peak starting from about 6° C., and an endothermic peak from about 120° C. According to DSC thermogram and observation by melting apparatus, the endothermic peak starting from about 120° C. is dehydration/desolvation accompanied with melting. No recrystallization was observed after melting. TGA shows about 7.9% weight loss at 70° C. and about 7.4% weight loss from 70° C. to 200° C. KF shows 9.6% water content (about 6.2 equivalent by molar ratio). ¹H-NMR shows 1.8 equivalent DMSO (about 12.0% by weight).

### Free Acid Form M

Compound I free acid Form M is a metastable hydrate. It was obtained from water by equilibration at 25° C. by using amorphous form as starting material. It converted to Pattern N with the addition of Pattern N seeds.

### Free Acid Form N

Compound I free acid Form N was prepared by equilibration of free acid Form A in DMSO/water (1:1 v/v) at 50° C. The XRPD of free acid Form N is shown in FIG. 17A. The DSC curve of free acid Form N is shown in FIG. 17B, which showed three endothermic peaks at 68.6° C., 81.0° C., and 207.7° C. The TGA curve of free acid Form N is shown in FIG. 17C, which showed a weight loss of 9.0% up to 200° C.

In summary, Compound I free acid Form A is the most stable polymorph in a wide water activity range from 0 to 0.8. It shows good chemical and physical stability and is only slightly hygroscopic. It shows reversible dehydration-hydration behavior upon heating and hydration kinetics is fast. Although it converts to another hydrate Form N in water, free acid Form A is stable in bulk at 92.5% RH up to 1 week. Therefore, Compound I free acid Form A is recommended as the optimal polymorph for further development.

### Abbreviation for Solvent Used

The solvent abbreviations are listed in Table 3-16.

TABLE 3-16
Solvent abbreviation list

| Abbreviation | Solvent |
|---|---|
| MeOH | Methanol |
| EtOH | Ethanol |
| IPA | Isopropyl alcohol |
| MIBK | 4-Methyl-2-pentanone |
| EtOAc | Ethyl acetate |
| IPAc | Isopropyl acetate |
| MTBE | Methyl tert-butyl ether |
| CHCl₃ | Chloroform |
| PG | Propylene glycol |
| THF | Tetrahydrofuran |
| 2-MeTHF | 2-Methyltetrahydrofuran |
| DCM | Dichloromethane |
| ACN | Acetonitrile |
| DMSO | Dimethylsulfoxide |
| DMAc | N,N-Dimethylacetamide |
| NMP | 1-Methyl-2-pyrrolidone |
| MEK | Methyl ethyl ketone |

### Instruments and Methods

### XRPD

XRPD data was collected on a Bruker D8 Advance X-Ray powder diffractometer. Detailed parameters are listed in Table 3-17.

TABLE 3-17
Parameters for XRPD test
X-ray Powder Diffractometer (XRPD)

| | |
|---|---|
| Instrument | Bruker D8 Advance |
| X-ray geometry | Reflection |
| Detector | LYNXEYE_XE_T (1D mode) |
| Open angle | 2.9° (max) |
| Radiation | Cu/K-Alpha1 (λ = 1.5406 Å) |
| X-ray generator power | 40 kV, 40 mA |
| Primary beam path slits | Twin Primary motorized slit: 20.0 mm by |
| | sample length; Primary Soller slit: 2.5° |
| Secondary beam path slits | Secondary Soller slit: 2.5° |
| Scan mode | Continuous scan |
| Scan type | Locked coupled |
| Step size | 0.02° |
| Time per step | 0.3 second per step |
| Scan range | 2° to 40° |
| Sample rotation speed | 15 rpm |
| Sample holder | Flat monocrystalline silicon |

### Variable Temperature XRPD

### XRPD

For Variable Temperature XRPD (VT-XRPD) analysis, Bruker D8 Advance X-ray powder diffractometer was used. The XRPD parameters used are listed in Table 3-18.

TABLE 3-18
Parameters for VT-XRPD test

| | |
|---|---|
| Instrument | Bruker D8 Advance |
| X-ray geometry | Reflection |
| Detector | LYNXEYE (1D mode) |
| Open angle | 2.1° (max) |
| Radiation | Cu/K-Alpha1 (λ = 1.5406 Å) |
| X-ray generator power | 40 kV, 40 mA |
| Primary beam path slits | Divergence fixed slit: 0.6 mm; |
| | Primary Soller slit: 2.5° |
| Secondary beam path slits | Secondary Soller slit: 2.5° |
| Scan mode | Continuous scan |
| Scan type | Locked coupled |
| Step size | 0.02° |
| Time per step | 0.6 second per step |
| Scan range | 4° to 40° |
| Non-ambient stage | CHC Plus⁺ chamber |

### DSC and TGA

DSC data were collected using a TA Discovery 2500 from TA Instruments. DSC was performed using a TA Discovery 5500 from TA Instruments. Detailed parameters used are listed in Table 3-19.

TABLE 3-19
Parameters for DSC and TGA test
Differential Scanning Calorimeter (DSC)

| | |
|---|---|
| Instrument | TA Discovery 2500 |
| Sample pan | Tzero pan and Tzero hermetic lid with |
| | a pin hole of 0.7 mm in diameter |
| Temperature range | 0 to 250° C. |
| | 0 to 280° C. |
| Heating rate | 10° C./min |
| Nitrogen flow | 50 mL/min |
| Sample mass | About 1-2 mg |

Thermal Gravimetric Analysis (TGA)

| | |
|---|---|
| Instrument | Discovery 5500 |
| Sample pan | Aluminum, open |
| Start temperature | Ambient condition (below 35° C.) |
| Final temperature | 300° C. |
| Heating rate | 10° C./min |
| Nitrogen flow | Balance 10 mL/min; sample |
| | chamber 25 mL/min |
| Sample mass | About 2-10 mg |

### DVS

DVS was measured via a SMS (Surface Measurement Systems) DVS Intrinsic or a ProUmid SPSx-1μ Advance. Detailed parameters for DVS test are listed in Table 3-20.

TABLE 3-20
Parameters for DVS test
Method 1 (for hydrates)

| | |
|---|---|
| Instrument | Intrinsic |
| Total gas flow | 200 sccm |
| Oven temperature | 25° C. |
| Solvent | Water |
| Method | Cycle: 40-95-0-95-40% RH |
| | Stage Step: 10% |
| | Equilibrium: 0.002 dm/dt (%/min ) |
| | Minimum dm/dt stability duration: 60 min |
| | Maximum dm/dt stage time: 360 min |
| Sample mass | About 10 mg |

Method 2 (for hydrates)

| | |
|---|---|
| Instrument | ProUmid SPSx-1μ Advance |
| Total gas flow | Max. 4,000 mL/min |
| Oven temperature | 25° C. |
| Solvent | Water |
| Method | Cycle: 40-95-0-95-40% RH |
| | Stage Step: 10% RH |
| | Equilibrium: 240 min for each step |
| Sample mass | ~5-50 mg |

### KF

Karl Fischer data was collected using a Coulometric KF Titrator C30 from Mettler Toledo or a Metrohm 851/885 system. Detailed parameters used are listed in Table 3-21.

TABLE 3-21
Parameters for KF test
Method 1

| | |
|---|---|
| Instrument | Mettler Toledo Coulometric KF Titrator C30 |
| Method | Coulometric |
| Sample mass | About 1-10 mg |

Method 2

| | |
|---|---|
| Instrument | Metrohm 851/885 system |
| Method | Coulometric |
| Oven temperature | 120° C. |
| Extraction time | 120 s |
| Nitrogen flow | 50 mL/min |
| Sample mass | ~5-30 mg |

### ¹H NMR

¹H NMR data was collected on a Bruker Avance-AV 400 MHz NMR using DMSO-d₆ as solvent. Detailed parameters used are listed in Table 3-22.

TABLE 3-22
Parameters for ¹H NMR measurements

| | |
|---|---|
| Instrument | Bruker Avance-AV 400M |
| Frequency | 400 MHz |
| Probe | 5 mm PABBO BB/19F-1H/D Z-GRD Z108618/0406 |
| Number of scan | 8 |
| Temperature | 297.6 K |
| Relaxation delay | 1 second |

### Example 4: Evaluation of Compound I Free Acid Form A

### Physicochemical Characteristics of Compound I Free Acid Form A

In order to obtain a representative sample without residual solvent, Compound I free acid Form A was prepared using the procedure below. This batch was used for the evaluation in this section.

About 500 mg of Compound I free acid Form A was weighed to a 20 mL of glass vial. 4 mL of EtOAc was added to the vial. Obtained suspension was stirred at 25° C. at a rate of 200 rpm. The suspension was kept stirring at 25° C. for about 2 days. Solids were collected by centrifugation and then placed at ambient condition (23-27° C., 50%-70% RH) for about 20 h. About 443 mg of free acid Form A was obtained (yield: 89%).

### Bulk Stability

Compound I free acid Form A, was placed at 25° C./92.5% RH in an open container, at 40° C./75% RH in an open container, and at 60° C. in a closed container for 1 week. Samples after storage were characterized by XRPD and HPLC and inspected for color change. The results are summarized in Table 4-1. No change in morphology or purity was observed for all 3 storage conditions.

TABLE 4-1
Summary of bulk stability

| | |
|---|---|
| Purity | Color |

| | |
|---|---|
| Initial purity | 97.8% |

Solid state, 25° C./92.5% RH, open container, 1 week

| | | |
|---|---|---|
| Bulk (HPLC) | 97.8% | No change of color |

| | |
|---|---|
| Bulk (XRPD) | No form change |

Solid state, 40° C./75% RH, open container, 1 week

| | | |
|---|---|---|
| Bulk (HPLC) | 97.7% | No change of color |

| | |
|---|---|
| Bulk (XRPD) | No form change |

Solid state, 60° C., tight container, 1 week

| | | |
|---|---|---|
| Bulk (HPLC) | 97.8% | No change of color |

| | |
|---|---|
| Bulk (XRPD) | No form change |

### Water Sorption and Desorption Experiments

Water sorption and desorption behavior of Compound I free acid Form A was investigated by DVS at 25° C. with a cycle of 40-95-0-95-40% RH, dm/dt 0.002, min. equilibration time 60 min, and max. equilibration time 360 min XRPD was measured after the DVS test to determine form change. The results are summarized in Table 4-2.

TABLE 4-2
Summary of DVS Tests

| | Method | | | |
|---|---|---|---|---|
| | 40-95-0-95-40% RH, dm/dt 0.002, min. equilibration | | | |
| | time 60 min, max. equilibration time 360 min, 25° C. | | | |
| Relative | 1^{st} desorp. | 1^{st} sorp. | 2^{nd} desorp. | 2^{nd} sorp. |
| humidity | Weight % | Weight % | Weight % | Weight % |
| at 25° C. | change | change | change | change |
| 0% | N/A | 0.0 | 0.0 | N/A |
| 10% | N/A | 0.8 | 1.1 | N/A |
| 20% | N/A | 1.5 | 1.4 | N/A |
| 30% | N/A | 1.8 | 1.7 | N/A |
| 40% | 2.0 | 2.0 | 2.0 | 2.0 |
| 50% | 2.2 | 2.2 | 2.3 | 2.2 |
| 60% | 2.4 | 2.4 | 2.5 | 2.4 |
| 70% | 2.5 | 2.5 | 2.6 | 2.5 |
| 80% | 2.6 | 2.6 | 2.7 | 2.7 |
| 90% | 2.8 | 2.9 | 2.8 | 2.8 |
| 95% | 3.0 | 3.0 | 2.9 | 2.9 |

XRPD after DVS test No form change
Hygroscopicity Slightly hygroscopic from 40% RH to 95% RH

### Compression Simulation Experiments

About 20 mg of Compound I free acid Form A was compressed for 5 minutes under 2.5 MPa, 5M Pa and 10 MPa with a hydraulic press. Potential form change and degree of crystallinity were evaluated by XRPD. The results are summarized in Table 4-3. Decrease in crystallinity was observed for all conditions but morphology remained unchanged.

TABLE 4-3
Summary of compression simulation experiments

| | |
|---|---|
| Pressure | XRPD |

| | | |
|---|---|---|
| 10 | MPa | No form change |
| | | Crystallinity decrease |
| 5 | MPa | No form change |
| | | Crystallinity slightly decrease |
| 2.5 | MPa | No form change |
| | | Crystallinity slightly decrease |

### Dry Grinding Simulation Experiments

About 20 mg of Compound I free acid Form A was ground manually with a mortar and a pestle for 3 min. No changes in form or degree of crystallinity were observed by XRPD.

### Wet Granulation Simulation Experiments

Water or ethanol was added drop wise to about 20 mg of Compound I free acid Form A until the sample was wetted sufficiently. Wet sample was ground gently with in a mortar and a pestle. Post granulation sample was dried under ambient condition for 10 min. The results are summarized in Table 4-4. No changes in form or degree of crystallinity were observed by XRPD for samples wetted with water. No form change and a slight decrease in crystallinity were observed by XRPD for the sample wetted with ethanol.

TABLE 4-4
Summary of wet granulation experiments

| Granulation solvents | XRPD |
|---|---|
| Water | No form change |
| | No obvious crystallinity decrease |
| Ethanol | No form change |
| | Crystallinity slightly decrease |

### Conclusion

Compound I free acid Form A was evaluated for bulk stability, hygroscopicity, compression simulation, and dry/wet granulation simulation experiments. Free acid Form A is physically and chemically stable after stressed at all 3 conditions over one week. No form change and no obvious chemical degradation was observed after bulk stability study. Free acid Form A is slightly hygroscopic from 40% RH to 95% RH with 1.0% water uptake. After the DVS test, obtained sample was still free acid Form A. Free acid Form A shows good tolerance to manually grinding and wet granulation by using water as dispersant with no form change and no obvious crystallinity decrease. Free acid Form A showed no form change and slight crystallinity decrease after wet granulation by using EtOH as dispersant and compression experiments under 2.5 MPa and 5 MPa. Free acid Form A showed no form change but obvious crystallinity decrease after compression under 10 MPa.

### Instruments and Methods

The XRPD and DVS instruments and methods used were the same as in Example 3.

### HPLC

HPLC data was collected on an Agilent 1260 InfinityII Binary Pump instrument. The detailed parameters are listed in Table 4-5.

TABLE 4-5
Parameters for HPLC
High Performance Liquid Chromatograph (HPLC)

| Instrument | Agilent 1260 infinityII Binary Pump | | |
|---|---|---|---|
| HPLC method | Wave length: 220 nm | | |
| | Column: Waters Xbridge C18, 4.6 × 150 mm, 3.5 μm | | |
| | Detector: DAD | | |
| | Column temperature: 45° C. | | |
| | Flow rate: 0.8 mL/min | | |
| | Mobile phase A: 5 mM NH₄OAc in water | | |
| | Mobile phase B: ACN:Methanol = 50:50(v/v) | | |
| | Diluent: ACN:Water = 70:30 (v/v) | | |
| | Injection volume: 5 μL | | |
| | Gradient: | | |
| | Time (min) | Mobile Phase A (%) | Mobile Phase B (%) |
| | Initial | 90 | 10 |
| | 10.00 | 47 | 53 |
| | 40.00 | 44 | 56 |
| | 50.00 | 5 | 95 |
| | 55.00 | 5 | 95 |
| | 56.00 | 90 | 10 |
| | 60.00 | 90 | 10 |

### Example 5: Additional Salt Screen Experiments

Additional salt screenings were performed with five counter ions as salt forming agents. Approximately 200 mg of Compound 1 free acid Form A was added to a suitable amount of acetone or ACN to get a clear solution. 1.0 equivalent of selected counter ions were dissolved in water. The solutions were mixed and were applied to rotary evaporation to obtain amorphous solids. Salt formation of obtained solids were confirmed by IR. Then the amorphous solids were equilibrated in different solvents to obtain crystalline salts. Acetone, ACN, THF, DCM and ethanol/water (1:1, v/v) were used as crystallization solvents. The results are summarized in Table 5-1. 2 crystalline salt hits (Na salt Form A and L-arginine salt Form A) were obtained, which were characterized by XRPD, TGA, DSC and NMR or HPLC/IC.

TABLE 5-1
Summary of Additional Salt Screening for Compound I

| | | | | | EtOH/water(1:1, |
|---|---|---|---|---|---|
| Counter ions | Acetone | ACN | THF | DCM | v:v) |
| Free Acid | Free Acid | Clear solution | Clear solution | Clear solution | Free Acid |
| Form A | Form A | | | | Form A |
| Sodium | Sodium salt | Sodium salt | Clear solution | Clear solution | PLM: No |
| hydroxide | Form A | Form A | | | birefringence |
| Potassium | Clear solution | Clear solution | Clear solution | Clear solution | Free Acid |
| hydroxide | | | | | Form A |
| Magnesium | Low | Low | Low | Clear solution | Free Acid |
| hydroxide | crystallinity | crystallinity | crystallinity | | Form A |
| Calcium | Low | Physical | Low | Clear solution | Physical |
| acetate | crystallinity | mixture (Free | crystallinity | | (mixture Free |
| | | Acid Form A + | | | Acid Form A + |
| | | calcium acetate) | | | calcium acetate) |
| L-arginine | L-arginine salt | Amorphous | L-arginine | PLM: No | Free Acid |
| | Form A | form | salt Form A | birefringence | Form A |

Clear solutions obtained from above were further cooled slowly to 5° C. No solids precipitated after stirring at 5° C. for 2 days. The solutions were further treated by antisolvent addition. The obtained suspensions were centrifuged to obtain solids, which were dried at 50° C. for 2 h under vacuum and analyzed by XRPD. The results are summarized in Table 5-2. Two new crystalline salt hits (potassium salt Form A and sodium salt Form B) were obtained.

TABLE 5-2
Salt Screening by antisolvent addition for Compound I

| Counter ions | Acetone | ACN | THF | DCM |
|---|---|---|---|---|
| Free acid | // | add toluene → | add toluene → | add heptane → |
| Form A | | clear solution | clear solution | Free acid Form A |
| Sodium | // | // | add water → | add heptane → |
| hydroxide | | | sodium salt | Amorphous |
| | | | Form B | |
| Potassium | add MTBE → | add toluene → | add toluene → | add heptane → gel |
| hydroxide | potassium | clear solution | clear solution | |
| | salt Form A | | | |
| Magnesium | // | // | // | add heptane → gel |
| hydroxide | | | | |
| Calcium | // | // | // | |
| hydroxide | | | | |

//: Not carried out

### Characterization of Compound I Salt Forms

Four salt forms were obtained from salt screening and further experiments, which were characterized by XRPD, TGA and DSC. The salt stoichiometry was determined using HPLC/IC or H NMR. All the characterization results were summarized in Table 5-3.

TABLE 5-3
Characterization Summary of Compound I Salts

| | Weight loss @ | Endotherm | Molar Ratio |
|---|---|---|---|
| Salt | 120° C. (%) | (° C., peak) | (base:FA) |
| Na salt Form A | 1.8 | 66.5 | 1.0 |
| Na salt Form B | // | // | // |
| K salt Form A | 1.4 | 50.9, 166.2, 237.7 | 0.9 |
| L-arginine salt | 2.4 | 57.3 | 1.0 |
| Form A | | | |

//: indicates not tested due to insufficient sample

### Na Salt

Compound I sodium salt Form A (Na salt Form A) was obtained via slurry of free acid Form A and equimolar NaOH in acetone at RT for 4 days.

The TGA/DSC curves of Compound I sodium salt Form A showed a weight loss of 1.8% up to 120° C. and an endotherm at 66.5° C. (peak). The ¹H NMR showed the molar ratio of acetone/API was 0.37 (2.3 wt %). HPLC/IC showed the molar ratio was 1.0 (base/FA).

Compound I sodium salt Form B (Na salt Form B) was obtained via slurry of free acid Form A and equimolar NaOH in THF at RT for 4 days, slow cooled to 5° C. and stirred for 2 days, followed by addition of antisolvent (water). The XRPD pattern is displayed in FIG. 19A.

### K Salt

Compound I potassium salt Form A (K salt Form A) was obtained via slurry of free acid Form A and equimolar KOH in acetone at RT for 4 days, slow cooled to 5° C. and stirred for 2 days, followed by addition of antisolvent (MTBE). The XRPD pattern is displayed in FIG. 20A.

The DSC curve of Compound I potassium salt Form A (K salt Form A) is displayed in FIG. 20B, which showed three endotherms at 50.9° C., 166.2° C., and 237.7° C. (peak). The TGA curve is displayed in FIG. 20C, which showed a weight loss of 1.4% up to 130° C. The ¹H NMR showed the molar ratio of MTBE/API was 0.45 (4.1 wt %) and the molar ratio of acetone/API was 0.04 (0.3 wt %). HPLC/IC results showed the molar ratio was 0.9 (base/FA).

### L-Arginine Salt

Compound I L-arginine salt Form A (L-arginine Form A) was obtained via slurry free acid Form A and equimolar L-arginine in THF at RT for 4 days.

The TGA/DSC curves of L-arginine salt Form A showed a weight loss of 2.4% up to 120° C. and an endotherm at 57.3° C. (peak). The ¹H NMR showed the molar ratio of L-arginine/API was 1.0 and the molar ratio of THF/API was 0.2 (1.5 wt %).

### Re-Preparation of Selected Salts

The Na salt Form A, Na salt Form B, and L-arginine salt Form A were selected for re-preparation. To prepare Na salt Form B, approximately 40 mg of free acid Form A and 1.0 equivalent sodium hydroxide was stirred in 0.6 mL water. After stirring at 50° C. for 4 days, the suspension was taken out and centrifuged. Solids obtained was analyzed by XRPD which showed that only the amorphous form was obtained.

The Na salt Form A and L-arginine salt Form A were successfully re-prepared at 600 mg scale and characterized by XRPD, TGA, DSC, NMR or HPLC/IC. The results are summarized in Table 5-4.

TABLE 5-4
Summary of characterization for re-prepared salts

| | | Endotherm | Molar ratio |
|---|---|---|---|
| Sample form | Weight loss (%) | (° C., peak) | (base/FA) |
| Na salt Form A | 4.1% up to 100° C.; | 124.8 | 1.0 |
| | 3.9% from 100° C. | | |
| | to 250° C. | | |
| L-arginine salt Form A | 3.2% up to 120° C. | 66.0 | 1.0 |

### Na Salt

Compound I sodium salt Form A (Na salt Form A) was re-prepared via slurry from approximately 600 mg free acid Form A and equimolar NaOH in 7.5 mL acetone at RT for 4 days. The obtained solid was centrifuged and dried at 50° C. for 2 h under vacuum. The XRPD pattern is displayed in FIG. 18A. The DSC curve of Na salt Form A is displayed in FIG. 18B which showed an endotherm at 124.8° C. (peak). The TGA curves of Na salt Form A is displayed in FIG. 18C which showed a weight loss of 4.1% up to 100° C. HPLC/IC results showed the molar ratio was 1.0 (base/FA). The ¹H NMR showed the molar ratio of acetone/API was 0.6 (3.8 wt %). Based on the data, Na salt Form A is likely an acetone and water hetero-solvate.

### L-Arginine Salt

Compound I L-arginine salt Form A (L-arginine salt Form A) was re-prepared via slurry from approximately 600 mg free acid Form A and equimolar L-arginine in 3.4 mL ACN/H₂O (7.5:1, v/v), followed by rotary evaporation and equilibration in THF at RT for 5 days. The obtained solid was centrifuged and dried at 50° C. for 2 h under vacuum. The XRPD pattern is shown in FIG. 1A. The DSC curve of L-arginine salt Form A is displayed in FIG. 1B, which showed an endotherm at 66.0° C. (peak). The TGA curve of L-arginine salt Form A is displayed in FIG. 1C, which showed a weight loss of 3.2% up to 120° C. The NMR showed the molar ratio of base/FA was 1.0 and the molar ratio of THF/API was 0.8 (5.6 wt %). Based on the data, L-arginine salt Form A is likely a THF and water hetero-solvate.

### Salt Evaluation

The two re-prepared salt samples were used for salt evaluation, including hygroscopicity, kinetic solubility and solid stability. The starting material Compound I free acid Form A was also evaluated for comparison.

### Solid Stability

Bulk stability of the free acid Form A and the two salt candidates were investigated at 25° C./92.5% RH in an open container, at 40° C./75% RH in a an open container and at 60° C. in a tight container over 2 weeks. The free acid Form A and the L-arginine salt Form A are chemically and physically stable under these conditions. The solid stability results are summarized in Table 5-5. The Na salt Form A is chemically stable in these conditions but physically unstable at 25° C./92.5% RH and 40° C./75% RH and showed form change at the two conditions as observed by XRPD.

TABLE 5-5
Summary of solid stability evaluation for Compound I

| Starting | | Form | HPLC | HPLC purity/ | Color |
|---|---|---|---|---|---|
| Material | Condition | change | purity (%) | initial (%) | Change |
| Free acid | Initial | — | 98.9 | — | — |
| Form A | 25° C./92.5% RH, open | No | 98.9 | 100 | No |
| | container, 2 weeks | | | | |
| | 40° C./75% RH, open | No | 98.4 | 99.5 | No |
| | container, 2 weeks | | | | |
| | 60° C., tight | No | 98.8 | 99.9 | No |
| | container, 2 weeks | | | | |
| Na salt | Initial | — | 99.0 | — | — |
| Form A | 25° C./92.5% RH, open | Yes* | 99.1 | >100 | No |
| | container, 2 weeks | | | | |
| | 40° C./75% RH, open | Yes* | 99.1 | >100 | No |
| | container, 2 weeks | | | | |
| | 60° C., tight | No | 99.1 | >100 | No |
| | container, 2 weeks | | | | |
| L-arginine | Initial | — | 98.8 | — | — |
| salt Form A | 25° C./92.5% RH, open | No | 98.5 | 99.6 | No |
| | container, 2 weeks | | | | |
| | 40° C./75% RH, open | No | 98.6 | 99.7 | No |
| | container, 2 weeks | | | | |
| | 60° C., tight | No | 98.5 | 99.6 | No |
| | container, 2 weeks | | | | |

*Likely isomorphic hydrate

### Kinetic Solubility

Kinetic solubility was measured for Compound I free acid Form A and re-prepared salt forms in water and three bio-relevant media. Table 4-11 summarizes the procedure for preparation of the bio-relevant media. Solubility of the free acid Form A and the 2 salt candidates was tested in 4 pH buffers (pH 1.2 HCl buffer, pH 4.5 acetate buffer, pH 8.0 alkaline borate buffer and water) and 3 bio-relevant media (SGF, FaSSIF-V1 and FeSSIF-V1) at 37 (C for 0.5 h and 1 h.

Around 10 mg material was weighed into 5 mL water, buffer solution, SGF, FaSSIF or FeSSIF¹ followed by rolling at 25 rpm at 37° C. for 0.5 and 1 h. At each time point, around 0.8 mL suspension was sampled out for centrifugation and filtration. Solids were tested by XRPD. The results are summarized in Table 5-6. Based on the results, the solubility of salts was higher than free acid Form A in water, alkaline borate buffer, and FeSSIF, and the solubility of the Na salt Form A was higher than that of L-arginine salt Form A. XRPD results showed disproportionation was observed for the Na salt Form A after solubility study.

TABLE 5-6
Summary of kinetic solubility test

| | | Free acid Form A | | | Na salt Form A | | | L-arginine salt Form A | | |
|---|---|---|---|---|---|---|---|---|---|---|
| | Time | Solubility | | | Solubility | | | Solubility | | |
| Solvent (pH) | (h) | (pH) | | XRPD | (pH) | | XRPD | (pH) | | XRPD |
| H₂O | 0.5 | 2.9 | (7.67) | FA | 1907.0 | (8.50) | // | 556.0 | (8.30) | LA |
| | 1.0 | 3.3 | (6.58) | FA | 1742.2 | (8.34) | // | 546.9 | (8.31) | LA |
| 0.1N HCl (1.2) | 0.5 | <LOQ | (1.04) | FA | <LOQ | (0.95) | AF | <LOQ | (1.07) | AF |
| | 1.0 | <LOQ | (1.03) | FA | <LOQ | (1.06) | AF¹ | <LOQ | (1.05) | AF |
| 50 mM acetate | 0.5 | <LOQ | (4.47) | FA | <LOQ | (4.60) | AF² | <LOQ | (4.52) | LA |
| buffer (4.5) | 1.0 | <LOQ | (4.51) | FA | <LOQ | (4.55) | AF | <LOQ | (4.55) | LA |
| alkaline borate | 0.5 | 17.4 | (7.86) | FA | 179.0 | (7.99) | AF³ | 159.7 | (7.91) | LA³ |
| buffer (8) | 1.0 | 24.9 | (7.85) | FA | 164.2 | (7.99) | AF³ | 146.3 | (8.00) | LA³ |
| SGF (2.0) | 0.5 | 37.2 | (1.98) | FA | 14.2 | (2.13) | AF¹ | 15.4 | (2.21) | AF¹ |
| | 1.0 | 39.1 | (2.05) | FA | 15.8 | (2.17) | AF¹ | 15.6 | (2.28) | AF¹ |
| FeSSIF (5.0) | 0.5 | 21.3 | (4.98) | FA⁵ | 42.7 | (4.98) | NA¹ | 38.5 | (4.95) | LA¹ |
| | 1.0 | 29.8 | (4.96) | FA⁵ | 40.9 | (5.06) | AF¹ | 37.0 | (4.94) | LA¹ |
| FaSSIF (6.5) | 0.5 | 7.5 | (6.50) | FA | 9.1 | (6.53) | AF³ | 7.2 | (6.51) | LA³ |
| | 1.0 | 8.2 | (6.47) | FA | 10.2 | (6.60) | AF³ | 7.7 | (6.50) | LA³ |

*LOQ = 0.500 μg/mL;
//: Not tested due to insufficient material
FA: Free acid Form A;
NA: Na salt Form A;
LA: L-arginine salt Form A;
AF: Amorphous Form
¹NaCl also detected.
²additional peak @ 2θ: 8.9°
³additional peak @ 2θ: 28.5°
⁵Similar to free acid Form A

### Hygroscopicity

In order to evaluate the hygroscopicity of Compound I free acid Form A and re-prepared salt forms (Na salt Form A and L-arginine salt Form A), DVS isotherm plots were collected at 25° C. between 0% RH and 95% RH. XRPD characterization was performed for the samples after DVS test. The DVS evaluation results are summarized in Table 5-7. Based on the results, all three forms are hygroscopic, and the lowest water uptake was observed for Compound I free acid Form A. Compound I Na salt Form A converted to a new form after DVS-test.

TABLE 5-7
Summary of DVS evaluation

| | Water uptake | Form change |
|---|---|---|
| Form | (@ 25° C./40-95% RH) | after DVS |
| Na salt Form A | 10.4% | Yes* |
| L-arginine salt Form A | 15.1% | No |
| Free acid Form A | 5.7% | No |

*converted to a potential isomorphic hydrate by XRPD

### Conclusion

Using Compound I free acid Form A as starting material, a salt screening was performed under 33 conditions using 11 bases in 3 solvent systems. No crystalline salt hits were obtained, and 2 new free acid forms were identified. Additional experiments using 6 bases in ACN/H2O (7:3 v/v) as solvent also did not yield any crystalline salt hits, but 4 new free acid forms were identified. Finally, additional experiments using 5 counterions and 5 solvents, followed by slow cooling and/or anti-solvent additions yielded 4 crystalline salt hits. Compound I sodium salt Form A, Compound I sodium salt Form B, and Compound I L-arginine salt Form A were selected for re-preparation. Out of the three, Compound I sodium salt Form A and Compound I L-arginine salt Form A were prepared successfully.

The re-prepared salts were used for salt evaluation along with Compound I free acid Form A. The DVS results showed that free acid Form A exhibited the lowest water uptake and form change was observed after DVS for Compound I Na salt Form A. The kinetic solubility results showed that the solubility of salts was higher than free acid Form A in water, alkaline borate buffer, and FeSSIF, and the solubility of the Na salt Form A was higher than that of L-arginine salt Form A. The results of solid stability evaluation showed the free acid Form A and the L-arginine salt Form A are chemically and physically stable under all tested conditions, but Na salt Form A is physically unstable at 25° C./92.5% RH and 40° C./75% RH.

Based on the salt evaluation and solid-state characterization results, Compound I L-arginine salt Form A was selected for further polymorph screening.

### Instruments and Methods

### XRPD

For XRPD analysis, Bruker X-ray powder diffractometers were used. The XRPD parameters used are listed in Table 5-8.

TABLE 5-8
Parameters for XRPD test
X-ray Powder Diffractometer (XRPD)

| | |
|---|---|
| Instrument | Bruker D8 Advance |

XRPD method 1(About 10 min, for sodium
salt Form A and L-arginine salt Form A)

| | |
|---|---|
| Detector | LYNXEYE_XE_T(1D mode) |
| Open angle | 2.94° |
| Scan mode | Continuous PSD fast |
| Radiation | Cu/K-Alpha1 (λ = 1.5418 Å) |
| X-ray generator power | 40 kV, 40 mA |
| Step size | 0.02° |
| Time per step | 0.3 second per step |
| Scan range | 2° to 40° |
| Primary beam path slits | Twin_Primary motorized slit 10.0 mm by |
| | sample length; SollerMount axial soller 2.5° |
| Secondary beam path slits | Detector OpticsMount soller slit 2.5°; |
| | Twin secondary motorized slit 5.2 mm |
| Sample rotation speed | 15 rpm |

XRPD method 2 (About 2 min, for salt screening samples)

| | |
|---|---|
| Detector | LYNXEYE_XE_T(ID mode) |
| Open angle | 2.94° |
| Scan mode | Continuous PSD fast |
| Radiation | Cu/K-Alpha1 (λ = 1.5418 Å) |
| X-ray generator power | 40 kV, 40 mA |
| Step size | 0.02° |
| Time per step | 0.06 second per step |
| Scan range | 3° to 40° |
| Primary beam path slits | Twin_Primary motorized slit 10.0 mm by |
| | sample length; SollerMount axial soller 2.5° |
| Secondary beam path slits | Detector OpticsMount soller slit 2.5°; |
| | Twin Secondary motorized slit 5.2 mm |
| Sample rotation speed | 15 rpm |

### TGA and DSC

TGA data were collected using a Discovery 5500 or Q5000 TGA from TA Instruments. DSC was performed using a Discovery 2500 DSC from TA Instruments. Detailed parameters used are listed in Table 5-9.

TABLE 5-9
Parameters for TGA and DSC test
Differential Scanning Calorimetric (DSC)

| | |
|---|---|
| Instrument | TA Discovery 2500 |
| Sample pan | Tzero pan and Tzero hermetic lid with a |
| | pin hole |
| Temperature range | 30 to 250° C. or before decomposition |
| Heating rate | 10° C./min |
| Nitrogen flow | 50 mL/min |
| Sample mass | ~1-2 mg |

Thermal Gravimetric Analysis (TGA)

| | |
|---|---|
| Instrument | Discovery 5500 or Q5000 |
| Sample pan | Aluminum, open |
| Nitrogen flow | Balance 10 mL/min; sample 25 mL/min |
| Start temperature | Ambient condition (below 35° C.) |
| Final temperature | 300° C. or abort next segment if weight <80% |
| | (w/w) |
| | (The weight loss of the compound is no more |
| | than 20% (w/w)) |
| Heating rate | 10° C./min |
| Sample mass | ~2-10 mg |

### DVS

DVS was measured via a SMS (Surface Measurement Systems) DVS Intrinsic. The relative humidity at 25° C. were calibrated against deliquescence point of LiCl, Mg(NO₃)₂ and KCl. Parameters for DVS test are listed in Table 5-10.

TABLE 5-10
Parameters for DVS test
Dynamic Vapor Sorption (DVS)

| | |
|---|---|
| Instrument | Intrinsic |
| Total gas flow | 200 sccm |
| Oven temperature | 25° C. |
| Solvent | Water |
| Method | Cycle: 40-95-0-95-40% RH |
| | Stage Step: 10% |
| | Equilibrium: 0.002 dm/dt (%/min) |
| | Minimum dm/dt stability duration: 60 min |
| | Maximum dm/dt stage time: 360 min |

### PLM

PLM picture was captured on Olympus BX53LED using a crossed polarizer, silicone oil added.

### Karl Fischer

KF analysis was performed on a Mettler Toledo Coulometric KF Titrator C30 using a Coulometric method.

### Solution NMR

Solution NMR was collected on Bruker Avance-AV 400 MHz NMR Spectrometer.

### HPLC/IC

Waters H-Class UPLC were utilized and detailed chromatographic conditions are listed in Table 5-11. IC parameters were listed in Table 5-12.

TABLE 5-11
HPLC methods for purity test

| Instrument | Shimadzu LC-20ADXR | | |
|---|---|---|---|
| Wave length | 254 nm | | |
| Column | Agilent Zorbax SB-C18 4.6 mm*150 mm | | |
| Detector: | DAD | | |
| Column temperature: | 40° C. | | |
| Flow rate | 1.2 mL/min | | |
| Injection volume | 5 μL | | |
| Diluent | ACN/water (1:1, v:v) | | |
| | Time | Mobile Phase A: 0.1% | Mobile phase B: |
| Gradient | (min) | TFA in water (%) | ACN (%) |
| | 0 | 95 | 5 |
| | 0.01 | 95 | 5 |
| | 9.0 | 5 | 95 |
| | 13.0 | 5 | 95 |
| | 13.1 | 95 | 5 |
| | 17.0 | 95 | 5 |

TABLE 5-12
IC parameters for stoichiometric test

| | |
|---|---|
| Instrument | Metrohm 940 professional IC |
| Sample center | 889 IC |
| Detector | Conductivity detector |
| Eluent (anion) | 3.2 mmol/L Na₂CO₃ + 1.0 mmol/L NaHCO₃ |
| Eluent (cation) | 2.5 mmol/L MSA |
| Suppressor solutions | 0.5% H₂SO₄ |
| Column: | Anion A SUPP 5-150 or Cation Column C4-150 |
| Column temperature: | 30° C. |
| Flow rate: | 0.7 mL/min (anion) or 0.9 mL/min (cation) |
| Diluent: | ACN/water (1:1, v:v) |
| Injection volume: | 20 μL |

### Example 6: Polymorph Screening and Evaluation for Compound I L-arginine Salt

The purpose of this project was to perform polymorph screening and evaluation for L-arginine salt to select a lead form for further study. The starting material characterized by XRPD, TGA and DSC. XRPD pattern showed the material was free acid Form A. The TGA/DSC results showed a weight loss of 3.8% up to 200° C. and a melting endotherm at 232.2° C. (onset temperature).

### Re-Preparation of L-Arginine Salt

Approximately 8 g of Compound I L-arginine salt Form B was prepared from the reaction of Compound I and L-arginine in IPA/H₂O (7:3 v/v). After stirring for ˜2 h at 50-55° C., 2.38 equivalents by volume IPA and 2 wt % crystal seed were added into the clear solution whereupon the mixture became hazy. After stirring for 2 h, ˜15.5 equivalents by volume IPA was added dropwise into the mixture. After stirring at 50-55° C. for 8 h, the mixture was further stirred at 0-5° C. for 10 h. The precipitated solid was collected by filtration, washed with IPA and dried at 65-75° C. Purity by HPLC: 97.7%.

### Approximate Solubility at 25° C. and at 50° C.

The solubility of the starting material was determined at 2 temperatures in 21 solvents.

For solubility at 25° C., approximately 5 mg of L-arginine salt was weighed to a 2 mL glass vial. 20 μL aliquots of each solvent were added to dissolve the substance at 25° C. Vortex and sonication were applied to assist dissolution. Max. volume of each solvent added was 1 mL. Approximate solubility was determined by visual observation.

For solubility at 50° C., approximately 10 mg of L-arginine salt was weighed to a 2 mL glass vial. 20 μL aliquots of each solvent were added to dissolve the substance at 50° C. Vortex and sonication were applied to assist dissolution. Max. volume of each solvent added was 1 mL. Approximate solubility was determined by visual observation. The results are summarized in Table 6-1.

TABLE 6-1
Summary of solubility experiments at 25° C. and 50° C.

| | Solubility (mg/mL) | | | |
|---|---|---|---|---|
| Solvents | 25° C. | Comment | 50° C. | Comment |
| Water | <5 | Decreased solids amount | <10 | Decreased solids amount |
| MeOH | 6-7 | — | 10-11 | — |
| EtOH | <5 | Decreased solids amount | <10 | Decreased solids amount |
| Acetone | <5 | — | <10 | — |
| ACN | <5 | — | <10 | — |
| THF | <5 | Decreased solids amount | <10 | Decreased solids amount |
| 2-MeTHF | <5 | — | <10 | — |
| 1,4-Dioxane | <5 | Fluffy solids | <10 | Fluffy solids |
| EtOAc | <5 | — | <10 | — |
| IPAc | <5 | — | <10 | — |
| MTBE | <5 | — | <10 | — |
| Heptane | <5 | — | <10 | — |
| DCM | <5 | Almost clear solution | // | — |
| DMSO | 62-83 | — | 125-166 | — |
| Toluene | <5 | Decreased solids amount | <10 | Decreased solids amount |
| MEK | <5 | — | <10 | — |
| Pyridine | <5 | Decreased solids amount | <10 | Decreased solids amount |
| DMF | 8-10 | — | 10-11 | — |
| NMP | <5 | Decreased solid amount, | 5-10 | Almost clear solution |
| | | placed at 50° C., clear | | |
| | | solution | | |
| CHCl₃ | <5 | Almost clear solution, | 5-10 | Almost clear solution |
| | | placed at 50° C., clear | | |
| | | solution | | |
| IPA | <5 | Placed at 50° C., no | <5 | — |
| | | obvious decrease on | | |
| | | solids amount | | |

//: Not carried out

Equilibration with solvents at 25° C. and 50° C.

Based on approximate solubility results, about 40 mg Compound I L-arginine salt was equilibrated in 0.2-1 mL of solvents at 25° C. for 2 weeks or 50° C. for 1 week while stirring at a rate of 300-400 rpm. Obtained suspensions were filtered through a 0.45 μm nylon membrane filter by centrifugation at 14,000 rpm. Solid precipitates (wet cakes) were investigated by XRPD. For samples with different XRPD patterns, additional analysis including DSC, TGA, ¹H-NMR, and KF for hydrates were performed. The results are summarized in Table 6-2 and 6-3.

TABLE 6-2
Summary of equilibration experiments at 25° C. for 2 weeks

| Solvents | XRPD | Additional analysis | Comments |
|---|---|---|---|
| MeOH | Free acid Form A | // | — |
| EtOH | L-arginine salt | DSC: Endothermic peak from | — |
| | Form B10 | about 8° C., Melting onset: | |
| | | 228.4° C. | |
| | | TGA: 3.3% @ 100° C. | |
| | | 1.9%@ 100-230° C. | |
| | | ¹H-NMR: 0.5 equiv. EtOH | |
| | | was detected (about 2.1% by | |
| | | weight) | |
| Acetone | L-arginine salt | ¹H-NMR: 0.2 equiv. Acetone | After dried under vacuum at |
| | Form C | (about 1.1% by weight) | 25° C. for about 18 h, |
| | | KF: 5.4% water by weight | ¹H-NMR: 0.005 equiv. Acetone |
| | | (equal to ~3.4 equiv. water) | (about 0.03% by weight) |
| ACN | L-arginine salt | ¹H-NMR: 0.1 equiv. ACN | After dried under vacuum at |
| | Form C | (about 0.5% by weight) | 25° C. for about 18 h, |
| | | KF: 6.3% water by weight | ¹H-NMR: 0.01 equiv. ACN |
| | | (equal to ~4.1 equiv. water) | (about 0.04% by weight) |
| THF | L-arginine salt | // | — |
| | Form B1 | | |
| 2-MeTHF | L-arginine salt | // | — |
| | Form B2 | | |
| 1,4-Dioxane | L-arginine salt | // | — |
| | Form B3 | | |
| EA | L-arginine salt | // | — |
| | Form B4 | | |
| IPAc | L-arginine salt | // | — |
| | Form B5 | | |
| DCM | Disproportionated | // | — |
| | to L-arginine | | |
| Toluene | L-arginine salt | ¹H-NMR: No detectable | — |
| | Form C | residual solvent | |
| Pyridine | L-arginine salt | // | — |
| | Form B6 | | |
| MEK | L-arginine salt | // | — |
| | Form B7 | | |
| DMF | L-arginine salt | // | — |
| | Form A1 | | |
| NMP | L-arginine salt | ¹H-NMR: 0.7 equiv. NMP | Heated to 120° C. then cooled to |
| | Form B8 | (about 5.9% by weight) | ambient condition (about 20-25° |
| | | 0.3 equiv. IPA (about 1.5% | C., 60-80% RH) |
| | | by weight) | XRPD: No form change |
| | | | DSC: Endothermic peak from |
| | | | about 116° C., Melting onset: |
| | | | 230.2° C. |
| | | | ¹H-NMR: 0.5 equiv. NMP |
| | | | (about 4.3% by weight) |
| | | | 0.2 equiv. IPA (about 1.0% by |
| | | | weight) |
| Chloroform | Disproportionated | // | — |
| | to L-arginine | | |
| MeOH/DCM | // | // | Clear solution |
| (v:v = 1:1) | | | |
| ACN/water | L-arginine salt | ¹H-NMR: 0.6 equiv. ACN | — |
| (v:v = 9:1) | Form A2 | (about 2.2% by weight) | |
| a.w. = 0.87 | | | |
| MeOH/water | Free acid Form A | // | — |
| (v:v = 1:1) | | | |
| a.w. = 0.76 | | | |
| DMSO/water | L-arginine salt | // | — |
| (v:v = 1:1) | Form B9 | | |
| a.w. = 0.68 | | | |
| Water | // | // | Gel |
| PG/MTBE | L-arginine salt | // | — |
| (v:v = 1:1) | Form B11 | | |
| IPA/Water | L-arginine salt | // | — |
| (v:v = 17:1) | Form B | | |

TABLE 6-3
Summary of equilibration experiments at 50° C. for 1 week

| Solvents | XRPD | Additional analysis | Comments |
|---|---|---|---|
| MeOH | Free acid Form A | // | — |
| EtOH | L-arginine salt | ¹H-NMR: No detectable | — |
| | Form C | residual solvent | |
| Acetone | L-arginine salt | ¹H-NMR: 0.03 equiv. acetone | — |
| | Form C | (about 0.2% by weight) | |
| | | KF: 5.7% water by weight | |
| | | (equal to ~3.7 equiv. water) | |
| ACN | L-arginine salt | ¹H-NMR: 0.04 equiv. ACN | — |
| | Form C | (about 0.2% by weight) | |
| THF | L-arginine salt | // | — |
| | Form B1 | | |
| 2-MeTHF | L-arginine salt | // | — |
| | Form B2 | | |
| 1,4-Dioxane | L-arginine salt | // | — |
| | Form B3 | | |
| EtOAc | L-arginine salt | // | — |
| | Form B4 | | |
| IPAc | L-arginine salt | // | — |
| | Form B5 | | |
| Toluene | L-arginine salt | DSC: Endothermic peak from | — |
| | Form B12 | about 11° C. | |
| | | Melting upon decomposition: | |
| | | 226.8° C. | |
| | | TGA: About 5.4% @ 200° C. | |
| | | 3.0% @ 200-250° C. | |
| | | ¹H-NMR: 0.5 equiv. toluene | |
| | | (about 4.0% by weight) | |
| | | KF: 6.9% (equal to ~4.7 equiv. | |
| | | water) | |
| Pyridine | L-arginine salt | // | — |
| | Form B6 | | |
| MEK | L-arginine salt | // | — |
| | Form B7 | | |
| DMF | L-arginine salt | // | — |
| | Form A1 | | |
| NMP | L-arginine salt | // | — |
| | Form B8 | | |
| Chloroform | Physical mixture | // | — |
| | of L-arginine salt | | |
| | and L-arginine | | |
| ACN/water | L-arginine salt | ¹H-NMR: 0.02 equiv. ACN | — |
| (v:v = 9:1) | Form C | (about 0.08% by weight) | |
| a.w. = 0.82 | | KF: 4.6% water by weight | |
| | | (equal to ~2.9 equiv. water) | |
| MeOH/water | Free acid Form A | // | — |
| (v:v = 1:1) | | | |
| a.w. = 0.76 | | | |
| DMSO/water | L-arginine salt | // | — |
| (v:v = 1:1) | Form B9 | | |
| a.w. = 0.70 | | | |
| Water | // | // | Gel |
| PG/MTBE | L-arginine salt | // | Deliquescent in ambient |
| (v:v = 1:1) | Form B11 | | condition (about 23-27° C., 60- |
| | | | 80% RH) |
| IPA/Water | L-arginine salt | ¹H-NMR: 0.9 equiv. IPA | — |
| (v:v = 17:1) | Form B | (about 4.7% by weight) | |

### Equilibration Under a Temperature Cycle

Based on approximate solubility results, approximately 50 mg of L-arginine salt was equilibrated in 0.2˜1 mL of solvent under a temperature cycle between 5° C. to 50° C. at a heating/cooling rate of 0.1° C./min for 10 cycles. The equilibration was executed with a stirring bar on a magnetic stirring plate at a rate of 6300-400 rpm.

Obtained suspensions were filtered through a 0.45 μm nylon membrane filter by centrifugation at 14,000 rpm. Solid parts (wet cakes) were investigated by XRPD. For samples with different XRPD patterns, additional analysis including DSC, TGA, ¹H-NMR, and KF for hydrate was performed. The results are summarized in Table 6-4.

TABLE 6-4
Summary of equilibration experiments under a temperature cycle

| Solvents | XRPD | Additional analysis | Comments |
|---|---|---|---|
| MeOH | Free acid Form A | // | — |
| EtOH | L-arginine salt | DSC: Dehydration from about 3° C., | VT-XRPD: |
| | Form C | Melting upon decomposition onset: | |
| | | 228.8° C. | |
| | | TGA: About 6.2% @ 200° C. | |
| | | 2.8% @200-250° C. | |
| | | ¹H-NMR: No detectable residual | |
| | | solvent | |
| | | KF: 6.0% water by weight (equal | |
| | | to ~3.9 equiv. water) | |
| Acetone | L-arginine salt | ¹H-NMR: 0.05 equiv. Acetone (about | — |
| | Form C | 0.3% by weight) | |
| | | KF: 5.0% water by weight (equal | |
| | | to ~3.1 equiv. water) | |
| ACN | L-arginine salt | ¹H-NMR: No detectable residual | — |
| | Form C | solvent | |
| | | KF: 6.0% water by weight (equal | |
| | | to ~3.9 equiv. water) | |
| THF | L-arginine salt | DSC: Endothermic peak from about | — |
| | Form B1 | 2° C. and 123° C., Melting upon | |
| | | decomposition: 227.2° C. | |
| | | TGA: 1.9% @ 100° C. | |
| | | 4.4% @ 100° C.-200° C. | |
| | | 2.9% @ 200-250° C. | |
| | | ¹H-NMR: 0.8 equiv. THF (about | |
| | | 5.0% by weight) | |
| | | KF: 5.2% (equal to ~3.5 equiv. water) | |
| 2-MeTHF | L-arginine salt | DSC: Endothermic peak from about | — |
| | Form B2 | 3° C. and 126° C., Melting upon | |
| | | decomposition: 226.0° C. | |
| | | TGA: 1.0%@ 100° C. | |
| | | 5.5%@ 100° C.-200° C. | |
| | | 3.2% @, 200-250° C. | |
| | | ¹H-NMR: 0.8 equiv. 2-Me-THF | |
| | | (About 5.8% by weight), | |
| | | 0.4 equiv. IPA (About 2.0% by | |
| | | weight) | |
| | | KF: 3.4% (equal to ~2.3 equiv. water) | |
| 1,4-Dioxane | L-arginine salt | DSC: Endothermic peak from about | — |
| | Form B3 | 2° C. and 153° C., Melting upon | |
| | | decomposition: 226.0° C. | |
| | | TGA: 1.4% @ 100° C. | |
| | | 3.1% @ 100-200° C. | |
| | | 5.2% @ 200-250° C. | |
| | | ¹H-NMR: 1.2 equiv. 1,4-dioxane | |
| | | (About 8.7% by weight), 0.4 equiv. | |
| | | IPA (About 2.0% by weight) | |
| | | KF: 2.4% (equal to ~1.7 equiv. water) | |
| EtOAc | L-arginine salt | DSC: Endothermic peak from about | — |
| | Form B4 | 4° C., peak onset 93.1° C., Melting | |
| | | upon decomposition: 227.9° C. | |
| | | TGA: 3.0% @ 85° C. | |
| | | 2.0% @85° C.-200° C. | |
| | | 2.8% @ 200-250° C. | |
| | | ¹H-NMR: 0.2 equiv. EtOAc (About | |
| | | 1.6% by weight) | |
| | | KF: 5.3% (equal to ~3.4 equiv. water) | |
| IPAc | L-arginine salt | DSC: Endothermic peak from about | — |
| | Form B5 | 3° C., peak onset 127° C., Melting | |
| | | upon decomposition: 226.5° C. | |
| | | TGA: 2.5% @100° C. | |
| | | 3.2% @ 100-200° C. | |
| | | 2.8% @ 200-250° C. | |
| | | ¹H-NMR: 0.8 equiv. IPAc | |
| | | (About 7.0% by weight) | |
| | | KF: 4.3% (equal to ~2.9 equiv. water) | |
| Toluene | L-arginine salt | ¹H-NMR: 0.06 equiv. toluene | — |
| | Form C | (About 0.5% by weight) | |
| | | KF: 5.2% water by weight (equal | |
| | | to ~3.3 equiv. water) | |
| Pyridine | L-arginine salt | DSC: Endothermic peak from about | — |
| | Form B6 | 9° C., Melting onset: 230.2° C. | |
| | | TGA: 0.9% @ 100° C. | |
| | | 8.0% @ 100-250° C. | |
| | | ¹H-NMR: 0.5 equiv. Pyridine | |
| | | (About 3.5% by weight) | |
| | | KF: 2.4% (equal to ~1.5 equiv. water) | |
| MEK | L-arginine salt | DSC: Endothermic peak from about | — |
| | Form B7 | 3° C. and 109° C., Melting upon | |
| | | decomposition: 223.8° C. | |
| | | TGA: 2.3%@ 100° C. | |
| | | 2.9% @ 100-200° C. | |
| | | 2.9% @ 200-250° C. | |
| | | ¹H-NMR: 0.6 equiv. MEK | |
| | | (About 3.8% by weight) | |
| | | KF: 3.8% (equal to ~2.5 equiv. water) | |
| DMF | L-arginine salt | DSC: Endothermic peak from about | After storage at 25° C./ |
| | Form A1 | 5° C., Melting upon decomposition: | 92.5% RH in an open |
| | | 214.5° C. | container for 25 days, |
| | | TGA: 3.5%@ 100° C. | ¹H-NMR: No detectable |
| | | 4.0%@ 100-215° C. | residual solvent |
| | | 3.5% @ 215-250° C. | |
| | | ¹H-NMR: 0.9 equiv. DMF | |
| | | (About 5.7% by weight) | |
| | | KF: 5.4% (equal to ~3.4 equiv. water) | |
| NMP | L-arginine salt | DSC: Endothermic peak from about | — |
| | Form B8 | 4° C. and 62° C., Melting upon | |
| | | decomposition: 231.2° C. | |
| | | TGA: 3.0% @ 100° C. | |
| | | 9.1%@ 100-250° C. | |
| | | ¹H-NMR: 0.5 equiv. NMP (About | |
| | | 4.2% by weight) | |
| | | 0.5 equiv. IPA (About 2.6% by | |
| | | weight) | |
| | | KF: 6.6% (equal to ~4.6 equiv. water) | |
| Chloroform | Physical mixture | // | — |
| | of L-arginine salt | | |
| | and L-arginine | | |
| ACN/water | L-arginine salt | DSC: Endothermic peak from about | — |
| (v:v = 9:1) | Form A2 | 5° C., Melting upon decomposition: | |
| | | 196.2° C. | |
| | | TGA: 7.5% @205° C. | |
| | | 2.8%@ 205-250° C. | |
| | | ¹H-NMR: 0.5 equiv. ACN (About | |
| | | 1.8% by weight) | |
| MeOH/water | Free acid Form A | // | — |
| (v:v = 1:1) | | | |
| DMSO/water | L-arginine salt | DSC: Endothermic peak from about | — |
| (v:v = 1:1) | Form B9 | 5° C. and 75° C., Melting upon | |
| | | decomposition: 229.7° C. | |
| | | TGA: 7.7% @ 100° C. | |
| | | 5.2% @ 100-200° C. | |
| | | 4.0%@ 200-250° C. | |
| | | ¹H-NMR: 2.4 equiv. DMSO (About | |
| | | 15.0% by weight) | |
| | | KF: 11.6% (equal to ~9.3 equiv. | |
| | | water) | |
| Water | // | // | Gel |
| PG/MTBE | L-arginine salt | // | Deliquescent in ambient |
| (v:v = 1:1) | Form B11 | | condition (about 23- |
| | | | 27° C., 60-80% RH) |
| IPA/Water | L-arginine salt | // | — |
| (v:v = 17:1) | Form B | | |

### Crystallization at Room Temperature by Slow Evaporation

Based on approximate solubility results, about 20 mg of L-arginine salt was dissolved in 0.1-15 mL of solvents. Obtained solutions was filtered through a 0.45 μm syringe nylon membrane filter. The clear solutions was slowly evaporated in ambient condition (about 23-27° C., 60-80% RH). Solid residues were investigated by XRPD. The results are summarized in Table 6-5.

TABLE 6-5
Summary of crystallization at room temperature by fast evaporation

| Solvents | XRPD | Comments |
|---|---|---|
| MeOH | Similar to free acid form A, | — |
| | low crystallinity | |
| DMSO | // | Sticky material |
| DMF | // | Clear solution, |
| | | Evaporated at room temperature (about 20- |
| | | 25° C.) under dry nitrogen flow |
| | | Low crystallinity, similar to L-arginine salt |
| | | Form A |
| Water | Amorphous form | — |
| MeOH/DCM | Amorphous form | — |
| (v:v = 1:1) | | |

### Crystallization at Room Temperature by Fast Evaporation

Based on approximate solubility results, approximately 20 mg L-arginine salt, was dissolved in 0.1-15 mL of solvents. Obtained solutions were filtered through a 0.45 μm syringe membrane filter. The clear solutions were fast evaporated at ambient condition (about 23-27° C., 60-80% RH) under a dry nitrogen flow. Solid residues were investigated by XRPD. The results are summarized in Table 6-6.

TABLE 6-6
Summary of crystallization at room temperature by fast evaporation

| Solvents | XRPD | Comments |
|---|---|---|
| MeOH | Amorphous form | — |
| DMSO | Amorphous form | — |
| DMF | Similar to L-arginine salt | — |
| | Form A1, low crystallinity | |
| Water | // | Few solids obtained due to low solubility |
| MeOH/DCM | Amorphous form | — |
| (v:v = 1:1) | | |

Crystallization from Hot Saturated Solutions by Slow Cooling

Based on approximate solubility results, about 30 mg of L-arginine salt, was dissolved in the minimal amount of selected solvents at 50° C. Obtained solutions were filtered through a 0.45 μm syringe membrane filter. The clear solutions were cooled to 5° C. at a rate of 0.1° C./min. Samples without precipitates at 5° C. were further cooled to −20° C. The results are summarized in Table 6-7.

TABLE 6-7
Summary of crystallization from hot
saturated solutions by slow cooling

| Solvents | Comments |
|---|---|
| MeOH | Clear solution |
| DMSO | Clear solution (cooled from |
| | 70° C. to 25° C.) |
| DMF | Clear solution |
| NMP | Clear solution |
| MeOH/water (v:v = 1:1) | Clear solution |
| MeOH/DCM (v:v = 1:1) | Clear solution |
| MeOH/THF (v:v = 1:1) | Clear solution |
| ACN/water (v:v = 1:1) | Clear solution |
| Acetone/water (v:v = 1:1) | Few solids, not enough for XRPD test |
| CHCl₃ | Clear solution |

Crystallization from Hot Saturated Solutions by Fast Cooling

Based on approximate solubility results, about 30 mg of L-arginine salt, was dissolved in the minimal amount of selected solvents at 50° C. Obtained solutions were filtered through a 0.45 μm syringe membrane filter. The clear solutions were put into 5° C. and agitated. Clear solutions were further cooled to −20° C. The results are summarized in Table 6-8.

TABLE 6-8
Summary of crystallization from hot saturated solutions by fast cooling

| Solvents | Comments |
|---|---|
| MeOH | Clear solution |
| DMSO | Clear solution (cooled from 70° C. to 25° C.) |
| DMF | Clear solution |
| NMP | Clear solution |
| MeOH/water | Few solids, not enough for XRPD |
| (v:v = 1:1) | Evaporated at ambient condition (about 23-27° C., 60-80% RH): |
| | Free acid Form A |
| MeOH/DCM | Clear solution |
| (v:v = 1:1) | Evaporated at ambient condition (about 23-27° C., 60-80% RH): |
| | Amorphous form |
| MeOH/THF | Clear solution |
| (v:v = 1:1) | Evaporated at ambient condition (about 23-27° C., 60-80% RH): |
| | Amorphous form |
| ACN/water | Clear solution |
| (v:v = 1:1) | Evaporated at ambient condition (about 23-27° C., 60-80% RH): |
| | Amorphous form |
| Acetone/water | Few solids, not enough for XRPD |
| (v:v = 1:1) | Evaporated at ambient condition (about 23-27° C., 60-80% RH): |
| | Low crystallinity, almost amorphous form |
| CHCl₃ | Clear solution |
| | Evaporated at ambient condition (about 23-27° C., 60-80% RH): |
| | Amorphous form |

### Crystallization by Addition of Anti-Solvent

Based on approximate solubility results, about 40 mg of L-arginine salt was dissolved in the minimal amount of selected good solvents at ambient condition (about 23-27° C., 60-80% RH). Obtained solutions were filtered through a 0.45 m syringe membrane filter. 4-8 times the volume of anti-solvent was added into the clear solutions slowly until a large amount of solids precipitated out.

Precipitates was collected by centrifugation filtration through a 0.45 am nylon membrane filter at 14,000 rpm. Solid parts (wet cakes) was investigated by XRPD. For samples with different XRPD patterns, additional analysis including ¹H-NMR were performed. The results are summarized in Table 6-9.

TABLE 6-9
Summary of crystallization by addition of anti-solvent

| Solvents | Anti-solvent | | | |
|---|---|---|---|---|
| (mL) | (mL) | XRPD | Additional analysis | Comments |
| MeOH (5) | Water (20) | // | // | Clear solution |
| MeOH (5) | MTBE (20) | Amorphous | After equilibration for 9 days, | — |
| | | form | similar to L-arginine salt Form A | |
| DMSO (0.4) | Water (1.6) | // | // | Clear solution |
| DMSO (0.35) | Acetone (1.4) | // | // | Clear solution |
| DMSO (0.35) | ACN (1.4) | L-arginine | After equilibration for 9 days, L- | — |
| | | salt Form A3 | arginine salt Form A3 | |
| | | ¹H-NMR: 4 | DVS: Absorbs about 4.6% water | |
| | | equiv. DMSO | from 40% RH to 95% RH at 25° C. | |
| | | | XRPD after DVS test: no form | |
| | | | change | |
| | | | ¹H-NMR after DVS test: 0.4 equiv. | |
| | | | DMSO | |
| DMSO (0.35) | Toluene (1.4) | Similar to L- | After equilibration for 9 days, L- | — |
| | | arginine salt | arginine salt Form A3 | |
| | | Form A3 | | |
| DMF (10) | Water (30) | // | // | Clear solution |
| DMF (10) | EtOH (30) | // | // | Clear solution |
| DMF (10) | ACN (30) | L-arginine | After equilibration for 9 days, L- | — |
| | | salt Form A1 | arginine salt Form Al | |
| Water (10) | EtOH (30) | // | // | Clear solution |
| Water (10) | MeOH (30) | // | // | Clear solution |

### Crystallization by Reverse Addition of Anti-Solvent

Based on approximate solubility results, about 40 mg of L-arginine salt, was dissolved in the minimal amount of selected good solvents at ambient condition (about 23-27 C, 60-80% RH). Obtained solutions was filtered through a 0.45 m syringe membrane filter. The clear solutions was added into 4-8 times the volume of anti-solvent quickly.

Precipitates were collected by centrifugation filtration through a 0.45 μm nylon membrane filter at 14,000 rpm. Solid parts (wet cakes) were investigated by XRPD. For samples with different XRPD patterns, additional analysis including TGA, DSC and ¹H-NMR were performed. The results are summarized in Table 6-10.

TABLE 6-10
Summary of crystallization by reverse addition of anti-solvent

| | Anti- | | | |
|---|---|---|---|---|
| Solvents(mL) | solvent(mL) | XRPD | Additional analysis | Comments |
| MeOH (5) | Water (20) | // | // | Clear solution |
| MeOH (5) | MTBE (20) | Amorphous | After equilibration for 9 | — |
| | | form | days, similar XRPD with | |
| | | | L-arginine salt Form A | |
| DMSO (0.4) | Water (1.6) | // | // | Clear solution |
| DMSO (0.35) | Acetone (1.4) | // | // | Clear solution |
| DMSO (0.35) | ACN (1.4) | L-arginine salt | After equilibration for 9 | Heated to 120° C. then |
| | | Form A3 | days, | cooled to ambient |
| | | | L-arginine salt Form A3 | condition (about 20- |
| | | | ¹NMR: 2.0 equiv. DMSO | 25° C., 60-80% RH) |
| | | | | XRPD: No form change |
| | | | | DSC: Endothermic peak |
| | | | | from about 8° C., |
| | | | | Melting onset: 213.5° C. |
| | | | | ¹H-NMR: 1.3 equiv. |
| | | | | DMSO |
| DMSO (0.35) | Toluene (1.4) | L-arginine salt | ¹H-NMR: 3.0 equiv. | — |
| | | Form A3 | DMSO | |
| DMF (10) | Water (30) | // | // | Clear solution |
| DMF (10) | EtOH (30) | // | // | Clear solution |
| DMF (10) | ACN (30) | L-arginine salt | After equilibration for 9 | — |
| | | Form A1 | days, L-arginine salt | |
| | | | Form A1 | |
| Water (10) | EtOH (30) | // | // | Clear solution |
| Water (10) | MeOH (30) | // | // | Clear solution |

### Crystallization by Vapor Diffusion

Based on approximate solubility results, about 30 mg of L-arginine salt, was dissolved in the minimal amount of selected solvents at about 20-25° C., 60-80% RH. Obtained solutions were filtered through a 0.45 m syringe membrane filter. The clear solutions were transferred into 4 mL glass vials without lids. Then these 4 mL vials were placed in 40 mL glass vials. To the 40 mL vials were added anti-solvents. Then these 40 mL vials were capped tightly and placed at about 23-27° C., 60-80% RH for up to 14 days. The results are summarized in Table 6-11.

TABLE 6-11
Summary of crystallization by vapor diffusion

| | Anti- | |
|---|---|---|
| Solvents(mL) | solvent(mL) | Comments |
| DMSO (0.4) | Acetone (1.6) | Yellow solution |
| DMSO (0.4) | ACN (1.6) | Hazy suspension |
| DMF (10) | Acetone (18) | Clear solution |
| DMF (8.5) | ACN (19) | Few solids, not enough for XRPD test |

### Crystallization by Heat-Cool DSC

Polymorphic behavior of L-arginine salt, was investigated by two different heat-cool DSC cycles. The results are summarized in Table 6-12.

TABLE 6-12
Summary of crystallization by heat-cool DSC

| Heat-cool cycles | Thermal events |
|---|---|
| Cycle 1: | Dehydration/desolvation |
| Step 1: 0° C. to 250° C. at 10° C./min; | from about 6° C. |
| Step 2: 250° C. to −20° C. at 20° C./min; | Melting onset: 227.3° C. |
| Step 3: reheat to 250° C. at 10° C./min. | Tg: 181.7° C. |
| Cycle 2: | Dehydration/desolvation |
| Step 1: 0° C. to 250° C. at 10° C./min; | from about 6° C. |
| Step 2: 250° C. to −20° C. at 2° C./min; | Melting onset: 228.0° C. |
| Step 3: reheat to 250° C. at 10° C./min. | Tg: 182.9° C. |

### Variable Temperature XRPD (VT-XRPD)

The interrelationship between the polymorphs was investigated by variable temperature XRPD. The results are summarized in Table 6-13.

TABLE 6-13
Summary of variable temperature XRPD (VT-XRPD)

| | Temperature | |
|---|---|---|
| Polymorphs | (equilibration time) | XRPD |
| L-arginine | 25° C. (initial) | L-arginine salt Form C |
| salt Form C | 120° C. (N₂) | L-arginine salt Form C1 |
| | 25° C. | L-arginine salt Form C |

### Conclusion

A total of 3 crystalline polymorphs of Compound I L-arginine salts were obtained from the screening experiments.

### L-Arginine Salt Form A

Compound I L-arginine salt Form A was prepared by the procedure given in Example 2, followed by equilibration in THF at RT for 5 days. The obtained solid was centrifuged and dried at 50° C. for 2 h under vacuum. The XRPD pattern is shown in FIG. 1A. The DSC curve of L-arginine salt Form A is displayed in FIG. 1B, which showed an endotherm at 66.0° C. (peak). The TGA curve of L-arginine salt Form A is displayed in FIG. 1C, which showed a weight loss of 3.2% up to 120° C.

### L-Arginine Salt Form B

Compound I L-arginine salt Form B was prepared by equilibration of the L-arginine salt starting material in IPA/water (17:1 v/v) at 25° C. The XRPD of L-arginine salt Form B is shown in FIG. 2A. The DSC curve of L-arginine salt Form B is shown in FIG. 2B, which showed an endotherm at 17.6° C. (peak) and 243.7° C. (peak). The TGA curve of free acid Form B is shown in FIG. 2C, which showed a weight loss of 3.9% up to 200° C. and additionally 4.5% from 200-250° C.

### L-Arginine Salt Form C

Compound I L-arginine salt Form C was prepared by equilibration of the L-arginine salt starting material in acetone 25° C. The XRPD of free acid Form B is shown in FIG. 3A. The DSC curve of free acid Form B is shown in FIG. 3B, which showed two endothermic peaks at 52.9° C. and 232.9° C. The TGA curve of free acid Form B is shown in FIG. 3C, which showed a weight loss 5.2 up to 200° C. and additionally 3.7% from 200-260° C.

### Instruments and Methods

### XRPD

For XRPD analysis, Bruker D8 Advance X-ray powder diffractometer was used. The XRPD parameters used are listed in Table 6-14.

TABLE 6-14
Parameters for XRPD test

| X-ray geometry | Reflection | |
|---|---|---|
| Detector | LYNXEYE_XE_T (1D mode) | |
| Open angle | 2.9° (max) | |
| Radiation | Cu/K-Alpha1 (λ = 1.5406 Å) | |
| X-ray generator power | 40 kV, 40 mA | |
| Primary beam path | Twin Primary motorized slit: | |
| slits | 10.0 mm by sample length; | |
| | Primary Soller slit: 2.5° | |
| Secondary beam path slits | Secondary Soller slit: 2.5° | |
| Scan mode | Continuous scan | |
| Scan type | Locked coupled | |
| Step size | 0.02° | |
| Time per step | 0.12 | second per step |
| Scan range | 3° to 40° | |
| Sample rotation speed | 15 | rpm |

| | |
|---|---|
| Sample holder | Flat monocrystalline silicon |

### Variable Temperature XRPD

### XRPD

For Variable Temperature XRPD (VT-XRPD) analysis, Bruker D8 Advance X-ray powder diffractometer was used. The XRPD parameters used are listed in Table 6-15.

TABLE 6-15
Parameters for VT-XRPD test

| | |
|---|---|
| Instrument | Bruker D8 Advance |
| X-ray geometry | Reflection |
| Detector | LYNXEYE (1D mode) |
| Open angle | 2.1° (max) |
| Radiation | Cu/K-Alpha1 (λ = 1.5406 Å) |
| X-ray generator power | 40 kV, 40 mA |
| Primary beam path slits | Divergence fixed slit: 0.6 mm; |
| | Primary Soller slit: 2.5° |
| Secondary beam path slits | Secondary Soller slit: 2.5° |
| Scan mode | Continuous scan |
| Scan type | Locked coupled |
| Step size | 0.02° |
| Time per step | 0.6 second per step |
| Scan range | 4° to 40° |
| Non-ambient stage | CHC Plus⁺ chamber |

### DSC and TGA

DSC data were collected using a TA Discovery 2500 from TA Instruments. DSC was performed using a TA Discovery 5500 from TA Instruments. Detailed parameters used are listed in Table 6-16.

TABLE 6-16
Parameters for DSC and TGA test
Differential Scanning Calorimeter (DSC)

| Instrument | TA Discovery 2500 | |
|---|---|---|
| Sample pan | Tzero pan and Tzero hermetic lid with a | |
| | pin hole of 0.7 mm in diameter | |
| Temperature range | 0 to 250° | C. |
| | 0 to 280° | C. |
| Heating rate | 10° | C./min |
| Nitrogen flow | 50 | mL/min |

| | |
|---|---|
| Sample mass | About 1-2 mg |

Thermal Gravimetric Analysis (TGA)

| Instrument | Discovery 5500 | |
|---|---|---|
| Sample pan | Aluminum, open | |
| Start temperature | Ambient condition (below 35° C.) | |
| Final temperature | 300° | C. |
| Heating rate | 10° | C./min |

| | |
|---|---|
| Nitrogen flow | Balance 10 mL/min; sample chamber 25 mL/min |
| Sample mass | About 2-10 mg |

### DVS

DVS was measured via a SMS (Surface Measurement Systems) DVS Intrinsic or a ProUmid SPSx-1μ Advance. Detailed parameters for DVS test are listed in Table 6-17.

TABLE 6-17
Parameters for DVS test
Method 1 (for hydrates)

| Instrument | Intrinsic | |
|---|---|---|
| Total gas flow | 200 | sccm |
| Oven temperature | 25° | C. |

| | |
|---|---|
| Solvent | Water |
| Method | Cycle: 40-95-0-95-40% RH |
| | Stage Step: 10% |
| | Equilibrium: 0.002 dm/dt (%/min ) |
| | Minimum dm/dt stability duration: 60 min |
| | Maximum dm/dt stage time: 360 min |
| Sample mass | About 10 mg |

Method 2 (for hydrates)

| Instrument | ProUmid SPSx-1μ Advance | |
|---|---|---|
| Total gas flow | Max. 4,000 mL/min | |
| Oven temperature | 25° | C. |
| Solvent | Water | |
| Method | Cycle: 40-95-0-95-40% RH | |
| | Stage Step: 10% RH | |
| | Equilibrium: 240 min for each step | |
| Sample mass | ~5-50 | mg |

### KF

Karl Fischer data was collected using a Coulometric KF Titrator C30 from Mettler Toledo or a Metrohm 851/885 system. Detailed parameters used are listed in Table 6-18.

TABLE 6-18
Parameters for KF test
Method 1

| | |
|---|---|
| Instrument | Mettler Toledo Coulometric KF Titrator C30 |
| Method | Coulometric |
| Sample mass | About 1-10 mg |

Method 2

| | |
|---|---|
| Instrument | Metrohm 851/885 system |
| Method | Coulometric |
| Oven temperature | 120° C. |
| Extraction time | 120 s |
| Nitrogen flow | 50 mL/min |
| Sample mass | ~5-30 mg |

### ¹H NMR

¹H NMR data was collected on a Bruker Avance-AV 400 MHz NMR using DMSO-d₆ as solvent. Detailed parameters used are listed in Table 6-19.

TABLE 6-19
Parameters for ¹H NMR measurements

| | |
|---|---|
| Instrument | Bruker Avance-AV 400M |
| Frequency | 400 MHz |
| Probe | 5 mm PABBO BB/19F-1H/D Z-GRD Z108618/0406 |
| Number of scan | 8 |
| Temperature | 297.6 K |
| Relaxation delay | 1 second |

### Example 7: Preparation and Evaluation of Compound I L-Arginine Salt Form C

### Preparation of Compound I L-Arginine Salt Form C

Compound I L-arginine salt Form C, was prepared using the following procedure: About 1 g of L-arginine salt was weighed to a 20 mL glass vial and equilibrated with 2 mL of acetone at 50° C. A suspension was obtained. About 5 mg of L-arginine salt Form C seed crystal was added to the above suspension. After stirring for 1 day, the suspension became thick. Another 3 mL of acetone was added. Solids were collected by centrifugation at 4000 rpm after stirring for another 2 days. The wet cake was dried under vacuum at 50° C. with humidity control (about 6500 RH). Approximately 711 mg of L-arginine salt Form C was obtained with a yield of 710.

### Physicochemical Characteristics

The physical and chemical characteristics of Compound I L-arginine salt Form C obtained from above was thoroughly investigated by XRPD, DSC/TGA, HPLC, ¹H NMR, KF, and Scanning electron microscopy (SEM). The results are summarized in Table 7-1.

TABLE 7-1
Summary of physicochemical characteristics of Compound I L-arginine salt Form C

| | Polymorph | |
|---|---|---|
| | L-arginine salt Form C | |
| Parameters | Method | Results |
| Purity | HPLC | 97.6% |
| X-ray diffraction | 3-40° (2 theta) | High crystallinity |
| Melting onset and | DSC, 10° C./min | Dehydration from about 7° C. |
| enthalpy | | Melting upon decomposition: 222.0° C. |
| Thermogravimetry | TGA, 10° C./min | About 5.2% @ 200° C. |
| | | About 3.7% @ 200-260° C. |
| Stoichiometry | ¹H-NMR (DMSO-d₆) | 0.02 equiv. acetone (about 0.1% by weight) |
| Water content for hydrate | Karl Fisher | 6.8% by weight (equal to ~4.4 equiv. water) |
| Morphology | SEM | Irregular particles, ranging from about 1 μm to 5 μm |

| | |
|---|---|
| Comments | Before drying: ¹H-NMR: 1.1 equiv. acetone (6.0% by weight) |
| | Dried under vacuum at 50° C. without humidity control: |
| | ¹H-NMR: 0.2 equiv. acetone (1.1% by weight) |
| | Dried under vacuum at 50° C. with humidity controlled |
| | to about 65% RH: 0.02 equiv. acetone (0.1% by weight) |

### Bulk Stability

L-arginine salt Form C was placed at 25° C./92.5% RH in an open container, at 40° C./75% RH in an open container, and at 60° C. in a closed container for 1 week. Samples after storage were characterized by XRPD and HPLC and inspected for color change. No changes in HPLC purity, color, or by XRPD were observed for all storage conditions. The results are summarized in Table 7-2.

TABLE 7-2
Summary of Compound I L-arginine salt Form C bulk stability

| | |
|---|---|
| Purity | Color |

| | |
|---|---|
| Initial purity | 97.6% |

Solid state, 25° C./92.5% RH, open container, 1 week

| | | |
|---|---|---|
| Bulk (HPLC) | 97.8% | No color change |

| | |
|---|---|
| Bulk (XRPD) | No form change |

Solid state, 40° C./75% RH, open container, 1 week

| | | |
|---|---|---|
| Bulk (HPLC) | 97.7% | No color change |

| | |
|---|---|
| Bulk (XRPD) | No form change |

Solid state, 60° C., tight container, 1 week

| | | |
|---|---|---|
| Bulk (HPLC) | 97.7% | No color change |

| | |
|---|---|
| Bulk (XRPD) | No form change |

### Water Sorption and Desorption Experiments

Water sorption and desorption behavior of L-arginine salt Form C was investigated by DVS and XRPD was measured after the DVS test to determine form change. The sample was slightly hygroscopic and about 4.3% water uptake was observed from 40% RH to 95% RH. The results are summarized in Table 7-3.

TABLE 7-3
Summary of water sorption and desorption experiments

| | Method | | | |
|---|---|---|---|---|
| | 40-95-0-95-40% RH, dm/dt 0.002, min. equilibration | | | |
| | time 60 min, max. equilibration time 360 min, 25° C. | | | |
| Relative | 1^{st} sorp. | 1^{st} desorp. | 2^{nd} sorp. | 2^{nd} desorp. |
| humidity | Wt % | Wt % | Wt % | Wt % |
| at 25° C. | change | change | change | change |
| 0% | N/A | 0.0 | 0.0 | N/A |
| 10% | N/A | 0.6 | 0.5 | N/A |
| 20% | N/A | 1.5 | 1.5 | N/A |
| 30% | N/A | 2.9 | 2.8 | N/A |
| 40% | 4.4 | 4.4 | 4.1 | 4.4 |
| 50% | 5.1 | 5.3 | 5.0 | 5.3 |
| 60% | 5.8 | 5.9 | 5.7 | 6.0 |
| 70% | 6.3 | 6.6 | 6.3 | 6.6 |
| 80% | 6.8 | 7.2 | 6.9 | 7.2 |
| 90% | 7.6 | 7.9 | 7.7 | 8.0 |
| 95% | 8.7 | 8.7 | 8.7 | 8.8 |

DVS
Slightly hygroscopic in <70% RH. About 4.3% water uptake from 40% RH to 95% RH.
XRPD after DVS test
No form change
KF after DVS test
5.2% water by weight (equal to ~3.3 equiv. water)

### Compression Simulation Experiments

About 20 mg of L-arginine salt Form C was compressed for 3 minutes under 2.5 MPa, 5 MPa and 10 MPa with a hydraulic press. Potential form change and degree of crystallinity were evaluated by XRPD. No form change was observed but a slight decrease in crystallinity was observed. The results are summarized in Table 7-4.

TABLE 7-4
Summary of compression simulation experiments

| | |
|---|---|
| Pressure | XRPD |

| | | |
|---|---|---|
| 2.5 | MPa | No form change, crystallinity slightly decreased |
| 5 | MPa | No form change, crystallinity slightly decreased |
| 10 | MPa | No form change, crystallinity slightly decreased |

### Dry Grinding Simulation Experiments

About 20 mg of L-arginine salt Pattern C was ground manually with a mortar and a pestle for 3 min. Potential form change and degree of crystallinity were evaluated by XRPD. No form change was observed but a slight decrease in crystallinity was observed.

### Wet Granulation Simulation Experiments

Water or ethanol was added drop wise to about 20 mg of L-arginine salt Pattern C until the sample was wetted sufficiently. Wet sample was ground gently with in a mortar and a pestle for 3 min. Post granulation sample was dried under ambient condition (about 20-25° C., 60-80% RH) for 10 min. Potential form change and degree of crystallinity were evaluated by XRPD. No form change was observed but a slight decrease in crystallinity was observed. The results are summarized in Table 7-5.

TABLE 7-5
Summary of wet granulation simulation experiments

| Granulation solvents | XRPD |
|---|---|
| Water | No form change, crystallinity decreased |
| Ethanol | No form change, crystallinity decreased |

### Instruments and Methods

The TGA, DSC, DVS, KF, and NMR instruments and methods used were the same as in Example 7.

### XRPD

XRPD data was collected on a Bruker D8 Advance X-Ray powder diffractometer. Detailed parameters are listed in Table 7-6.

TABLE 7-6
Parameters for XRPD test
X-ray Powder Diffractometer (XRPD)

| | |
|---|---|
| Instrument | Bruker D8 Advance |
| X-ray geometry | Reflection |
| Detector | LYNXEYE_XE_T (1D mode) |
| Open angle | 2.9° (max) |
| Radiation | Cu/K-Alpha1 (λ = 1.5406 Å) |
| X-ray generator power | 40 kV, 40 mA |
| Primary beam path slits | Twin_Primary motorized slit: 20.0 mm by |
| | sample length; Primary Soller slit: 2.5° |
| Secondary beam path slits | Secondary Soller slit: 2.5° |
| Scan mode | Continuous scan |
| Scan type | Locked coupled |
| Step size | 0.02° |
| Time per step | 0.3 second per step |
| Scan range | 2° to 40° |
| Sample rotation speed | 15 rpm |
| Sample holder | Flat monocrystalline silicon |

### SEM

SEM images were collected on a Phenom Prox SEM-EDS instrument. Detailed parameters are listed in Table 7-7.

TABLE 7-7
Parameters for SEM
Scanning Electronic Microscope (SEM)

| | |
|---|---|
| Instrument | Phenom Prox SEM-EDS |
| Detector | BSD Full |
| Magnification | 200x-10000x |

### HPLC

HPLC data was collected on an Agilent 1260 InfinityII Binary Pump instrument. The detailed parameters are listed in Table 7-8.

TABLE 7-8
Parameters for HPLC
High Performance Liquid Chromatograph (HPLC)

| Instrument | Agilent 1260 infinityII Binary Pump | | |
|---|---|---|---|
| HPLC method | Wave length: 220 nm | | |
| | Column: Waters Xbridge C18, 4.6 × 150 mm, 3.5 μm | | |
| | Detector: DAD | | |
| | Column temperature: 45° C. | | |
| | Flow rate: 0.8 mL /min | | |
| | Mobile phase A: 5 mM NH₄OAc in water | | |
| | Mobile phase B: ACN:Methanol = 50:50(v/v) | | |
| | Diluent: ACN:Water = 70:30 (v/v ) | | |
| | Injection volume: 5 μL | | |
| Gradient: | Time (min) | Mobile Phase A (%) | Mobile Phase B (%) |
| | Initial | 90 | 10 |
| | 10.00 | 47 | 53 |
| | 40.00 | 44 | 56 |
| | 50.00 | 5 | 95 |
| | 55.00 | 5 | 95 |
| | 56.00 | 90 | 10 |
| | 60.00 | 90 | 10 |

### Example 8: Dog Pharmacokinetic Studies for Compound I L-Arginine Salt Form B, L-Arginine Salt Form A, Free Acid Form A, and Sodium Salt Form A

The objective of this study was to characterize the pharmacokinetics (PK) of Compound I in male Beagle dogs after oral (PO) or IV administration.

### Compound I Free Acid Form A—PO/TV

LC-MS/MS for the Compound I Free Acid Form A PK study was carried out using the following instruments/conditions

TABLE 8-1
LC-MS/MS for Compound I Free Acid Form A PO/IV PK Study

| LC-MS/MS Instrument | | |
|---|---|---|
| Autosampler | SIL-30AC (Shimadzu, Japan) | |
| HPLC equipment | LC-30AD (Shimadzu, Japan) | |
| MS/MS instrument | Triple Quad 6500+ (AB Sciex, USA) | |
| LC-MS/MS Conditions | | |
| Column | Waters X-Bridge BEH C18 | |
| | (2.1 × 50 mm, 2.5 μm) | |
| Mobile Phase | A: H₂O-0.025% FA-1 mM NH₄OAc | |
| | B: ACN-0.025% FA-1 mM NH₄OAc | |
| | Time (min) | % B |
| | 0.30 | 2 |
| | 1.00 | 90 |
| | 1.50 | 90 |
| | 1.51 | 2 |
| | 2.00 | Stop |

| | |
|---|---|
| Flow rate | 0.80 mL/min |
| Column Temperature | 50° C. |
| Injection Volume | 2 μL |
| Ion Source | Turbo Spray |
| Ionization Model | ESI |
| Scan Type | MRM |
| Ionization Mode | Positive |

Compound I Free Acid Form A was formulated as a 1 mg/mL solution in 1000 Solutol HS15+90% saline and administered orally (PO) by gavage at a target dose level of 2 mg/kg, or intravenously (IV) at a target dose level of 1 mg/kg.

The formulation was prepared on the day of dosing and stored at room temperature prior to administration. The dose was administered via cephalic vein injection and oral gavage at target dose volume of 1 and 2 mL/kg, respectively. Following intravenous and oral administration, blood samples were collected from the cephalic vein. For IV and PO groups, blood samples were collected at pre-dose, 0.083, 0.25, 0.5, 1, 2, 4, 8 and 24 h post dose.

The plasma concentration of Compound I Free Acid Form A was determined by liquid chromatography with mass spectrometric detection (LC-MS/MS). The PK data are summarized in Table 8-2 below.

TABLE 8-2
Summary of Mean PK parameters of
Compound I Free Acid Form A PO/IV

| | | Cₘₐₓ | Tₘₐₓ | AUCₗₐₛₜ |
|---|---|---|---|---|
| Polymorph | Dose | (ng/mL) | (h) | (h*ng/ml) |
| Free Acid Form A | IV, 1 mg/kg | N/A | N/A | 10644 |
| | PO, 2 mg/kg | 1790 | 1.0-2.0 | 9437 |

AUCₗₐₛₜ t = 24 h for PO/IV administration.
Tₘₐₓ was presented as range.

### Compound I L-Arginine Salt Form B—PO Formulations

LC-MS/MS for the Compound I L-arginine salt Form B PK study was carried out using the following instruments/conditions

TABLE 8-3
LC-MS/MS for Compound I L-arginine salt
Form B PO Formulations PK Study

| LC-MS/MS Instrument | | |
|---|---|---|
| Autosampler | SIL-30AC (Shimadzu, Japan) | |
| HPLC equipment | LC-30AD (Shimadzu, Japan) | |
| MS/MS instrument | Triple Quad 5500 (AB Sciex, USA) | |
| LC-MS/MS Conditions | | |
| Column | YMC-Triart Phenyl (2.1 × 50 mm, 3 μm) | |
| Mobile Phase | A: 5% ACN/95% H₂O-0.1% FA | |
| | B: 95% ACN/5% H₂O-0.1% FA | |
| | Time (min) | % B |
| | 0.01 | 20.0 |
| | 0.20 | 20.0 |
| | 1.90 | 100 |
| | 2.20 | 100 |
| | 2.21 | 20.0 |
| | 2.50 | 20.0 |

| | |
|---|---|
| Flow rate | 0.60 mL/min |
| Column Temperature | 25° C. |
| Injection Volume | 15 μL |
| Ion Source | Turbo Spray |
| Ionization Model | ESI |
| Scan Type | MRM |
| Ionization Mode | Positive |

Compound I L-arginine salt Form B was formulated as an oral HPMC capsule (POA) and as an oral solution in 10% TPGS in water (POB). The target dose was 2 mg/kg for both formulations.

The formulation was prepared on the day of dosing and stored at room temperature prior to administration. The dose was administered orally by gavage to dogs. Following oral administration, blood samples were collected from the animals via peripheral vein at 0.25, 0.5, 1, 2, 4, 8 and 24 h post dose.

The plasma concentration of Compound I L-arginine salt Form B was determined by liquid chromatography with mass spectrometric detection (LC-MS/MS). The PK data are summarized in Table 8-4 below.

TABLE 8-4
Summary of Mean PK parameters of Compound
I L-arginine salt Form B PO Formulations

| | | Cₘₐₓ | Tₘₐₓ | AUCₗₐₛₜ |
|---|---|---|---|---|
| Polymorph | Dose | (ng/mL) | (h) | (h*ng/mL) |
| L-arginine salt | Capsule (POA), 2 mg/kg | 328 | 2.0 | 1150 |
| Form B | | | [1.0-2.0] | |
| | Oral solution (POB), 2 mg/kg | 158 | 1.0 | 458 |
| | | | [1.0, 1.0, 1.0] | |

AUCₗₐₛₜ t = 8 or 24 h.
Tₘₐₓ was presented as median and range.

### Compound I Free Acid Form A, Sodium Salt Form A, and L-Arginine Salt Form A

LC-MS/MS for the PK study was carried out using the instruments and conditions shown in Table 8-3.

Compound I Free Acid Form A, sodium salt Form A, and L-arginine salt Form A, were formulated as an oral HPMC capsule (PO) at a target dose of 2 mg/kg.

The dose was administered orally by gavage to dogs. Following oral administration, blood samples were collected from the animals via peripheral vein at 0.25, 0.5, 1, 2, 4, 8 and 24 h post dose.

The plasma concentration of Compound I was determined by liquid chromatography with mass spectrometric detection (LC-MS/MS). The PK data are summarized in Table 8-5 below.

TABLE 8-5
Summary of Mean PK parameters of Compound I

| | | Cₘₐₓ | Tₘₐₓ | AUCₗₐₛₜ |
|---|---|---|---|---|
| Polymorph | Dose | (ng/mL) | (h) | (h*ng/m:) |
| Free Acid Form A | Capsule (PO), | 2.96 | 4.0 | 10.6 |
| | 2 mg/kg | | [2.0-4.0] | |
| Sodium salt Form A | | 64.1 | 2.0 | 198 |
| | | | [1.0-2.0] | |
| L-arginine salt Form A | | 68.8 | 2.0 | 289 |
| | | | [1.0-2.0] | |

AUCₗₐₛₜ t = 6 h for Free Acid Form A, t = 8 h for sodium salt Form A, and t = 24 h for L-arginine salt Form A.
Tₘₐₓ was presented as median and range.

### Conclusion PGP-102; I

Compound I L-arginine salt Form B showed excellent pharmacokinetic profiles. The mean AUCₗₐₛₜ and Cₘₐₓ values of Compound I L-arginine salt Form B in capsule was higher than that of Compound I Free Acid Form A, sodium salt Form A, or L-arginine salt Form A. Thus Compound I L-arginine salt Form B demonstrated better oral bioavailability over the other forms tested.

One skilled in the art would readily appreciate that the present disclosure is well adapted to obtain the ends and advantages mentioned, as well as those inherent therein. The methods, variances, and compositions described herein as presently representative of embodiments are exemplary and are not intended as limitations on the scope of the disclosure. Changes therein and other uses will occur to those skilled in the art, which are encompassed within the spirit of the disclosure, are defined by the scope of the claims.

1. 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one L-arginine salt (Compound I L-arginine salt), having the formula IB: 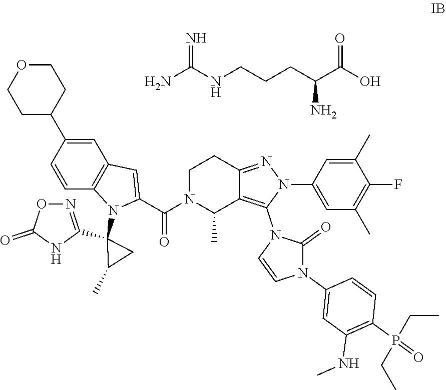 [SMILES: CCP(=O)(CC)c1ccc(-n2ccn(-c3c4c(nn3-c3cc(C)c(F)c(C)c3)CCN(C(=O)c3cc5cc(C6CCOCC6)ccc5n3[C@@]3(c5noc(=O)[nH]5)C[C@@H]3C)[C@H]4C)c2=O)cc1NC ; N=C(N)NCCC[C@H](N)C(=O)O]
or solvate thereof.

2. A crystalline form of 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one L-arginine salt (Compound I L-arginine salt), or solvate thereof.

3. Crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one L-arginine salt Form B (Compound I L-arginine salt Form B), characterized by an X-ray powder diffractogram comprising the following peaks expressed in ±0.2 degrees 2-theta selected from: 6.1, 7.4, and 10.3 as determined on a diffractometer using Cu-Kα radiation.

4. The crystalline Compound I L-arginine salt Form B of claim 3, further characterized by an X-ray powder diffractogram comprising one or more additional peaks expressed in ±0.2 degrees 2-theta selected from: 10.8, 15.3, 15.5, 18.0, 20.6, and 22.8 as determined on a diffractometer using Cu-Kα radiation.

5. The crystalline Compound I L-arginine salt Form B of claim 3, further characterized by an X-ray powder diffractogram as substantially shown in FIG. 2A.

6. The crystalline Compound I L-arginine salt Form B of claim 3, further characterized by a DSC comprising an endotherm at about 17.6° C. (peak) and about 243.7° C. (peak).

7. The crystalline Compound I L-arginine salt Form B of claim 3, further characterized by or a DSC as substantially shown in FIG. 2B.

8. Crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one L-arginine salt Form A (Compound I L-arginine salt Form A), characterized by an X-ray powder diffractogram comprising the following peaks expressed in ±0.2 degrees 2-theta selected from: 5.3, 9.1, and 11.5 as determined on a diffractometer using Cu-Kα radiation.

9. The crystalline Compound I L-arginine salt Form A of claim 8, further characterized by an X-ray powder diffractogram comprising one or more additional peaks expressed in ±0.2 degrees 2-theta selected from: 13.8, 15.9, 16.5, 18.9, 20.9, and 22.8 as determined on a diffractometer using Cu-Kα radiation; an X-ray powder diffractogram as substantially shown in FIG. 1A; a DSC comprising a broad endotherm at about 66.0° C. (peak) and about 35.8° C. (onset); or a DSC as substantially shown in FIG. 1B.

10. Crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one L-arginine salt Form C (Compound I L-arginine salt Form C), characterized by an X-ray powder diffractogram comprising the following peaks expressed in ±0.2 degrees 2-theta selected from: 6.1, 7.4, and 10.3 as determined on a diffractometer using Cu-Kα radiation.

11. The crystalline Compound I L-arginine salt Form C of claim 10, further characterized by an X-ray powder diffractogram comprising one or more additional peaks expressed in ±0.2 degrees 2-theta selected from: 10.8, 15.3, 15.5, 18.0, 20.6, and 22.8 as determined on a diffractometer using Cu-Kα radiation; an X-ray powder diffractogram as substantially shown in FIG. 3A; a DSC comprising a peak at about 52.9° C. (peak) and about 232.9° C. (peak); or a DSC as substantially shown in FIG. 3B.

12. Crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one (Compound I free acid Form A), characterized by an X-ray powder diffractogram comprising the following peaks expressed in ±0.2 degrees 2-theta selected from: 5.2, 6.1, and 12.4, as determined on a diffractometer using Cu-Kα radiation.

13. The crystalline Compound I free acid Form A of claim 12, further characterized by an X-ray powder diffractogram comprising one or more additional peaks expressed in ±0.2 degrees 2-theta selected from: 15.0, 16.5, 16.9, 18.8, 20.2, and 21.9 as determined on a diffractometer using Cu-Kα radiation; an X-ray powder diffractogram as substantially shown in FIG. 4A; a DSC comprising a peak at about 49.7° C. (peak) and about 211.3° C. (peak), or a DSC as substantially shown in FIG. 4B.

14. Crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one (Compound I free acid Form B), characterized by an X-ray powder diffractogram comprising the following peaks expressed in ±0.2 degrees 2-theta selected from: 7.8, 9.2, and 10.0, as determined on a diffractometer using Cu-Kα radiation.

15. The crystalline Compound I free acid Form B of claim 14, further characterized by an X-ray powder diffractogram comprising one or more additional peaks expressed in ±0.2 degrees 2-theta selected from: 10.3, 13.0, 13.7, 16.5, 20.5, and 23.2 as determined on a diffractometer using Cu-Kα radiation; an X-ray powder diffractogram as substantially shown in FIG. 5A; a DSC comprising a peak at about 32.4° C. (peak) and about 199.0° C. (peak), or a DSC as substantially shown in FIG. 5B.

16. Crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one (Compound I free acid Form C), characterized by an X-ray powder diffractogram comprising the following peaks expressed in ±0.2 degrees 2-theta selected from: 4.1, 8.1, and 10.4 as determined on a diffractometer using Cu-Kα radiation.

17. The crystalline Compound I free acid Form C of claim 16, further characterized by an X-ray powder diffractogram comprising one or more additional peaks expressed in ±0.2 degrees 2-theta selected from: 13.5, 14.6, 15.0, 15.5, 15.8, and 20.8 as determined on a diffractometer using Cu-Kα radiation; an X-ray powder diffractogram as substantially shown in FIG. 6A; a DSC comprising a peak at about 31.7° C. (peak), 134.9° C. (peak) and about 194.7° C. (peak), or a DSC as substantially shown in FIG. 6B.

18. Crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one (Compound I free acid Form D), characterized by an X-ray powder diffractogram comprising the following peaks expressed in ±0.2 degrees 2-theta selected from: 6.5, 12.1, and 12.9 as determined on a diffractometer using Cu-Kα radiation.

19. The crystalline Compound I free acid Form D of claim 18, further characterized by an X-ray powder diffractogram comprising one or more additional peaks expressed in ±0.2 degrees 2-theta selected from: 14.0, 16.5, 16.9, 17.5, 18.8, and 21.0 as determined on a diffractometer using Cu-Kα radiation; an X-ray powder diffractogram as substantially shown in FIG. 7A; a DSC comprising a peak at about 36.1° C. (peak), about 133.7° C. (peak), about 198.1° C. (peak), and about 223.9° C. (peak), or a DSC as substantially shown in FIG. 7B.

20. Crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one (Compound I free acid Form E), characterized by an X-ray powder diffractogram comprising the following peaks expressed in ±0.2 degrees 2-theta selected from: 5.7, 11.1, and 16.1 as determined on a diffractometer using Cu-Kα radiation.

21. The crystalline Compound I free acid Form E of claim 20, further characterized by an X-ray powder diffractogram comprising one or more additional peaks expressed in ±0.2 degrees 2-theta selected from: 17.1, 18.1, 18.7, 21.0, 21.3, and 21.6 as determined on a diffractometer using Cu-Kα radiation; an X-ray powder diffractogram as substantially shown in FIG. 8A; a DSC comprising a peak at about 43.6° C. (peak) and about 223.9° C. (peak), or a DSC as substantially shown in FIG. 8B.

22. Crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one (Compound I free acid Form F), characterized by an X-ray powder diffractogram comprising the following peaks expressed in ±0.2 degrees 2-theta selected from: 7.2, 12.9, and 14.6 as determined on a diffractometer using Cu-Kα radiation.

23. The crystalline Compound I free acid Form F of claim 22, further characterized by an X-ray powder diffractogram comprising one or more additional peaks expressed in ±0.2 degrees 2-theta selected from: 16.1, 16.6, 17.5, 19.1, 19.9, and 21.8 as determined on a diffractometer using Cu-Kα radiation; an X-ray powder diffractogram as substantially shown in FIG. 9A; a DSC comprising a peak at about 46.2° C. (peak), about 121.0° C. (peak), about 159.4° C. (peak), and about 230.4° C. (peak), or a DSC as substantially shown in FIG. 9B.

24. Crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one (Compound I free acid Form G), characterized by an X-ray powder diffractogram comprising the following peaks expressed in ±0.2 degrees 2-theta selected from: 6.0, 11.9, and 14.8 as determined on a diffractometer using Cu-Kα radiation.

25. The crystalline Compound I free acid Form G of claim 24, further characterized by an X-ray powder diffractogram comprising one or more additional peaks expressed in ±0.2 degrees 2-theta selected from: 16.3, 16.6, 18.4, 18.8, 21.3, and 23.9 as determined on a diffractometer using Cu-Kα radiation; an X-ray powder diffractogram as substantially shown in FIG. 10A; a DSC comprising a peak at about 52.9° C. (peak) and about 208.7° C. (peak), or a DSC as substantially shown in FIG. 10B.

26. Crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one (Compound I free acid Form H), characterized by an X-ray powder diffractogram comprising the following peaks expressed in ±0.2 degrees 2-theta selected from: 5.3, 5.5, and 7.6 as determined on a diffractometer using Cu-Kα radiation.

27. The crystalline Compound I free acid Form H of claim 26, further characterized by an X-ray powder diffractogram comprising one or more additional peaks expressed in ±0.2 degrees 2-theta selected from: 11.0, 11.3, 11.9, 14.4, 16.5, and 18.1 as determined on a diffractometer using Cu-Kα radiation; an X-ray powder diffractogram as substantially shown in FIG. 11A; a DSC comprising a peak at about 51.3° C. (peak), about 105.5° C. (peak), and about 217.2° C. (peak), or a DSC as substantially shown in FIG. 11B.

28. Crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one (Compound I free acid Form I), characterized by an X-ray powder diffractogram comprising the following peaks expressed in ±0.2 degrees 2-theta selected from: 4.0, 12.2, and 14.0 as determined on a diffractometer using Cu-Kα radiation.

29. The crystalline Compound I free acid Form I of claim 28, further characterized by an X-ray powder diffractogram comprising one or more additional peaks expressed in ±0.2 degrees 2-theta selected from: 15.1, 15.8, 16.7, 18.4, 21.0, and 22.0 as determined on a diffractometer using Cu-Kα radiation; an X-ray powder diffractogram as substantially shown in FIG. 12A; a DSC comprising a peak at about 41.5° C. (peak) and about 207.3° C. (peak), or a DSC as substantially shown in FIG. 12B.

30. Crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one (Compound I free acid Form J), characterized by an X-ray powder diffractogram comprising the following peaks expressed in ±0.2 degrees 2-theta selected from: 6.8, 11.6, and 13.5 as determined on a diffractometer using Cu-Kα radiation.

31. The crystalline Compound I free acid Form J of claim 30, further characterized by an X-ray powder diffractogram comprising one or more additional peaks expressed in ±0.2 degrees 2-theta selected from: 14.0, 15.0, 17.0, 17.3, 17.9, and 19.7 as determined on a diffractometer using Cu-Kα radiation; an X-ray powder diffractogram as substantially shown in FIG. 13A; a DSC comprising a peak at about 68.6° C. (peak) and about 221.2° C. (peak), or a DSC as substantially shown in FIG. 13B.

32. Crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one (Compound I free acid Form K), characterized by an X-ray powder diffractogram comprising the following peaks expressed in ±0.2 degrees 2-theta selected from: 8.7, 9.9, and 12.5 as determined on a diffractometer using Cu-Kα radiation.

33. The crystalline Compound I free acid Form K of claim 32, further characterized by an X-ray powder diffractogram comprising one or more additional peaks expressed in ±0.2 degrees 2-theta selected from: 14.2, 15.8, 16.4, 19.6, 21.1, and 23.6 as determined on a diffractometer using Cu-Kα radiation; an X-ray powder diffractogram as substantially shown in FIG. 14A; a DSC comprising a peak at about 46.1° C. (peak) and about 198.4° C. (peak), or a DSC as substantially shown in FIG. 14B.

34. Crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one (Compound I free acid Form N), characterized by an X-ray powder diffractogram comprising the following peaks expressed in ±0.2 degrees 2-theta selected from: 4.6, 6.3, and 7.2 as determined on a diffractometer using Cu-Kα radiation.

35. The crystalline Compound I free acid Form N of claim 34, further characterized by an X-ray powder diffractogram comprising one or more additional peaks expressed in ±0.2 degrees 2-theta selected from: 9.2, 11.9, 16.1, 18.6, 20.4, and 21.0 as determined on a diffractometer using Cu-Kα radiation; an X-ray powder diffractogram as substantially shown in FIG. 17A; a DSC comprising a peak at about 68.6° C. (peak), about 81.0° C. (peak), and about 207.7° C. (peak), or a DSC as substantially shown in FIG. 17B.

36. A crystalline salt form of 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one (Compound I), or solvate thereof, having the formula IA: 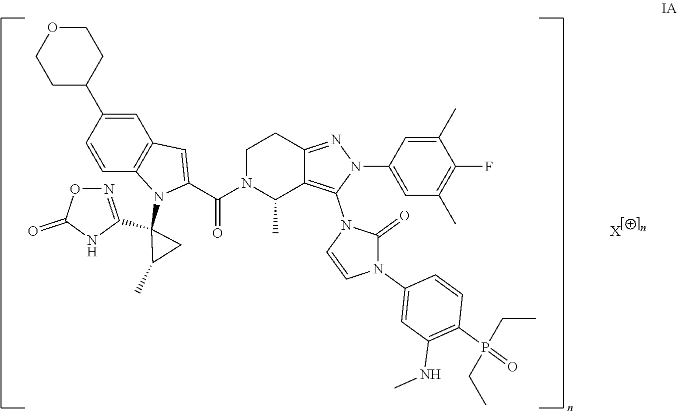
wherein: X is sodium and n is 1; X is potassium and n is 1, X is calcium and n is 2, or X is magnesium and n is 2.

37. The crystalline salt form of claim 36, wherein the crystalline salt form is selected from the group consisting of Compound I sodium salt Form A, Compound I sodium salt Form B, and Compound I potassium salt Form A.

38. A pharmaceutical composition comprising the Compound I L-arginine salt, or solvate thereof, of claim 1 and a pharmaceutically acceptable excipient.

39. A pharmaceutical composition comprising Compound I L-arginine salt, or solvate thereof, wherein at least 99% of Compound I is the Compound I L-arginine salt, or solvate thereof, of claim 1.

40. A pharmaceutical composition comprising Compound I L-arginine salt, or solvate thereof, wherein at least 99% of Compound I is the crystalline form of Compound I L-arginine salt, or solvate thereof, of claim 2.

41. A pharmaceutical composition comprising Compound I L-arginine salt, or solvate thereof, wherein at least 99% of Compound I is the crystalline form of Compound I L-arginine salt Form B of claim 3.

42. A pharmaceutical composition comprising Compound I L-arginine salt, or solvate thereof, wherein at least 99% of Compound I is the crystalline form of Compound I L-arginine salt Form A of claim 8.

43. A pharmaceutical composition comprising Compound I L-arginine salt, or solvate thereof, wherein at least 99% of Compound I is the crystalline form of Compound I L-arginine salt Form C of claim 10.

44-45. (canceled)

46. A method for treating a disease, disorder, or condition, in which modulation of repressed or impaired and/or elevated or unwanted GLP-1R is beneficial for the treatment of the underlying pathology and/or symptoms and/or progression of the disease, disorder, or condition, comprising administering to a subject in need thereof an effective amount of a pharmaceutical composition of claim 38.

47. The method of claim 46, wherein the disease, disorder, or condition is selected from the group consisting of type 1 diabetes mellitus, type 2 diabetes mellitus, early onset type 2 diabetes mellitus, idiopathic type 1 diabetes mellitus (Type 1b), youth-onset atypical diabetes (YOAD), maturity onset diabetes of the young (MODY), latent autoimmune diabetes in adults (LADA), obesity, weight gain from use of other agents, idiopathic intracranial hypertension, Wolfram syndrome, gout, excessive sugar craving, hypertriglyceridemia, dyslipidemia, malnutrition-related diabetes, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, traumatic brain injury, peripheral vascular disease, endothelial dysfunction, impaired vascular compliance, vascular restenosis, thrombosis, hypertension, pulmonary hypertension, restenosis after angioplasty, intermittent claudication, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, macular degeneration, cataract, glomerulosclerosis, arthritis, osteoporosis, treatment of addiction, cocaine dependence, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), ulcerative colitis, inflammatory bowel disease, colitis, irritable bowel syndrome, Crohn's disease, short bowel syndrome, Parkinson's, Alzheimer's disease, impaired cognition, schizophrenia, Polycystic Ovary Syndrome (PCOS), or any combination thereof.

48. The method of claim 47, wherein the disease, disorder, or condition, is type 2 diabetes mellitus.

49. A method of treating type 2 diabetes mellitus in a patient in need thereof, the method comprising administering to the patient a therapeutically effective amount of a pharmaceutical composition of claim 38.

50. A method for modulating insulin levels in a patient in need of such modulating, the method comprising administering to the patient an effective amount of a pharmaceutical composition of claim 38.

51. The method of claim 50, wherein the modulation results in an increase of insulin levels.

52. A method for modulating glucose levels in a patient in need of such modulating, the method comprising administering to the patient an effective amount of a pharmaceutical composition of claim 38.

53. The method of claim 52, wherein the modulation results in a decrease of glucose levels.

54. A process for preparing crystalline 3-((1S,2S)-1-(2-((S)-3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one L-arginine salt (Compound I L-arginine salt), comprising contacting Compound I with L-arginine in a solvent for a time sufficient to provide a crystalline Compound I L-arginine salt.

55. The process of claim 54, wherein the solvent is a mixture of IPA/H₂O.

56. The process of claim 54, wherein the contacting comprises adding 1.1 molar equivalents of L-arginine to Compound I at a temperature of about 10° C. to about 90° C.

57. The process of claim 54, wherein the contacting further comprises adding about 2 wt % of seed crystals to the mixture of Compound I and L-arginine.

58. The process of claim 54, wherein the contacting further comprises adding additional IPA dropwise into the mixture of Compound I and L-arginine and stirring at a temperature of about −10° C. to about 15° C.

59. The process of claim 54, wherein the process further comprises, following said contacting step, isolating the crystalline Compound I L-arginine salt.

60. The process of claim 59, wherein the isolating comprises the steps of filtering, washing, and drying the crystalline Compound I L-arginine salt.

61. The process of claim 54, wherein at least about 95% of the crystalline Compound I L-arginine salt is Form B.
